(19) United States
(12) Patent Application Publication
(10) Pub. No.: US 2023/0059204 A1
(43) Pub. Date: Feb. 23, 2023

(54) TRANSDERMAL MICRO-DOSING DELIVERY OF PHARMACEUTICAL AGENTS

(71) Applicant: PIKE THERAPEUTICS, INC., Vancouver BC (CA)

(72) Inventors: Fotios M. Plakogiannis, Whitestone, NY (US); Nisarg Modi, Jersey City, NJ (US)

(21) Appl. No.: 17/879,846

(22) Filed: Aug. 3, 2022

Related U.S. Application Data
(60) Provisional application No. 63/229,015, filed on Aug. 3, 2021.
(60) Provisional application No. 63/324,288, filed on Mar. 28, 2022.

(51) Int. Cl. A61K 9/70; A61K 31/352; A61K 31/05; A61K 31/675; A61K 31/4045; A61K 31/48; A61K 31/55; A61K 9/00

(52) U.S. Cl. CPC A61K 9/7046; A61K 31/352; A61K 31/05; A61K 31/675; A61K 31/4045; A61K 31/48; A61K 31/55; A61K 9/0014

(57) ABSTRACT

The present disclosure relates to the transdermal administration of pharmaceutical agents, such as CBD, THC, psilocybin, psilocin, lysergic acid diethylamine (LSD), and/or ibogaine, and derivatives of these compounds, for the treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, and cancer related or other end-of-life psychological distress.

This application claims benefit of Provisional application U.S. Ser. No. 63/229,015 filed Aug. 3, 2021 and Provisional U.S. Ser. No. 63/324,288 filed Mar. 28, 2022, the entireties of which are incorporated herein by reference.

### SPECIFICATION

### Background

The present disclosure relates to the transdermal administration of pharmaceutical agents, such as cannabidiol (CBD), tetrahydrocannabinol (THC), psilocybin, psilocin, lysergic acid diethylamine (LSD), and/or ibogaine, and derivatives of these compounds, for the treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, and cancer related or other end-of-life psychological distress.

The pharmaceutical agents, such as CBD, THC, psilocybin, psilocin, lysergic acid diethylamine (LSD), and/or ibogaine, and derivatives of these compounds may be used concomitantly with one or more other active pharmaceutical ingredients. Alternatively, the pharmaceutical agents, such as CBD, THC, psilocybin, psilocin, lysergic acid diethylamine (LSD), and/or ibogaine, and derivatives of these compounds may be formulated for administration separately, sequentially or simultaneously with one or more drugs or the combination may be provided in a single dosage form. Where pharmaceutical agents, such as CBD, THC, psilocybin, psilocin, lysergic acid diethylamine (LSD), and/or ibogaine, and derivatives of these compounds are formulated for administration separately, sequentially or simultaneously it may be provided as a kit or together with instructions to administer the one or more components in the manner as disclosed herein.

According to preliminary literature research, the maximum dose of psilocybin used in clinical trial is 0.6 mg/kg which is approximately 50 mg/70 kg. Furthermore, the lowest dose used in clinical trial was 1-3 mg/70 kg healthy volunteers. The present disclosure is directed to targeting, for example, 5 or 10 mg/day of active agent, such as for example psilocin, CBD, THC, psilocybin, psilocin, lysergic acid diethylamine (LSD), and/or ibogaine (depending upon the ability of API to penetrate through the skin) delivery through the transdermal route.

There are several approaches that utilize these psychedelics, including:
1. Micro-dosing involves very small daily doses of the psychedelic. It could be twice a week, or potentially daily.
2. Larger single dose to treat more problematic mental illness like treatment resistant depression, which is administered under physician supervision and can last 6 to 8 hours.

In one aspect a transdermal matrix patch or transdermal semisolid formulation containing for example, pharmaceutical agents, such as CBD, THC, psilocybin, psilocin, lysergic acid diethylamine (LSD), and/or ibogaine, and derivatives of these compounds can be prepared. In another aspect, two separate transdermal matrix patches can be prepared one containing pharmaceutical agent alone and a second containing another pharmaceutical agent alone as active ingredient. In this case both transdermal matrix patches could be applied at the same time and deliver pharmaceutical agent. In yet another exemplary aspect two separate transdermal semisolid formulations can be prepared one containing pharmaceutical agent alone and a second containing pharmaceutical agent alone as active ingredient. In this case both transdermal semisolid formulations could be applied at the same time and deliver pharmaceutical agents, such as CBD, THC, psilocybin, psilocin, lysergic acid diethylamine (LSD), and/or ibogaine, and derivatives of these compounds.

It is projected that mental health disorders are growing in every country and will cost the global economy $16 trillion by 2030 affecting nearly 2 billion people every year. (The Lancet Commission on global mental health and sustainable development).

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. Cannabinoids have immunomodulatory and immunosuppressive properties, suggesting these drugs as potential therapeutics in chronic inflammatory disease. Furthermore, cannabinoids receptors have been recently proposed as therapeutic targets for autoimmune disease including MS. The cannabis preparations can also be useful for chronic inflammatory conditions such as inflammatory bowel disease, rheumatoid arthritis, neurodegenerative disorders, and even in acute inflammation due to SARS-CoV-2 infections¹¹⁻¹⁵.

From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannbinol (CBN), cannabichromene (CBC), cannabigerol (CBG), tetrahydrocannbivarin (THCV), delta 9-tetrahydrocannbinol (delta9THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. In clinical studies Sativex (cannabinoid extract oromucosal spray containing THC and CBD) has shown improvements in neuropathic pain and sleep quality. Currently, Sativex is available as an oromucosal spary, which delivers 2.7 mg THC and 2.5 mg CBD per spray. The current dosage regimen is to spray the formulation in mouth for 8-10 times a day. According to patent, EP1361864B9, the inventor made an argument that the oral cannabis delivery will metabolize 90% of the dose. Furthermore, they also suggested that the mucous membrane of the buccal cavity, under the tongue and the nasopharynx are not metabolizing cannabis and delivering them directly into the blood stream by avoiding first pass metabolism. During Phase-I clinical study, it was found that sublingual and buccal application is only 18% and 11% higher than the oral administration. This study concluded that the oromucosal spray will increase the bioavailability of cannabinoids from 6% to 8-10%. There is a lot of cannabinoids which are metabolizing through first pass metabolism. Furthermore, the application of oromucosal spary need to be standardized with relation to food intake in order to minimize the variability of bioavailability in the individual patients. (www.medicines.org.uk/emc/product/602/smpc#gref) In other words, the variability in the bioavailability through the oromucosal spary is very high. (pubmed.ncbi.nlm.nih.gov/23052407/. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation (Costa, B. The non-psychoactive cannabis constituent cannabidiol is an orally effective therapeutic agent in rat chronic inflammatory and neuropathic pain. European Journal of Pharmacology. Volume 556, Issues 1-3, 5 Feb. 2007, Pages 75-83).

Transdermal delivery of pharmaceutical agents, such as CBD, THC, psilocybin, psilocin, lysergic acid diethylamine (LSD), and/or ibogaine, and derivatives of these compounds has a therapeutic potential for the management of, for example, MS. Currently, Sativex is given 8-10 sprays/day which is equivalent to 20 mg/day for CBD and THC individually. Upon consideration of bioavailability, the transdermal dose will be 2 mg/day.

The required flux for transdermal dose would be:

$$\begin{matrix}Required Flux = 2000 ug / 24 Hr / 50 \\ = 17 ug / sqcm / hr\end{matrix}$$

Furthermore, side effects related to oral delivery can be avoided using transdermal route. Furthermore, the peak and valley in the plasma concentration due to oral administration can be avoided by delivering the drug molecule constantly at predetermined input rate using transdermal dosage forms.

There are numerous patents available on cannabidiol, but the utility of those patents is not evaluated. One of the examples is the U.S. Pat. No. 9,375,417B2. According to patent '5417, the inventors provided examples, but they failed to provide any in-vitro or in-vivo data for those examples. Due to lack of these data, the utility of patent is unfeasible.

Another example of these patents is U.S. Pat. No. 6,328,992B1. This patent provides all the examples for reservoir and adhesive matrix patches. All these examples contain mixture of cannabinoids (such as delta-8-THC, delta-9-THC, cannabidiol and cannabinol) instead of cannabidiol only. The THC is psychoactive agent and addictive substance. So, the utility of patent is problematic.

In the US, Substance Abuse and Mental Health Services Administration (SAMHSA) study indicates that every year, about 42.5 million Americans suffer from some mental illness, including conditions such as depression, addiction, anxiety, substance abuse, etc. In addition, about 9.3 American adults suffer from a serious mental illness which impedes day to day activities like going to work. Among the indications for which administration of Where psychedelics, such as CBD, THC, psilocybin, psilocin, lysergic acid diethylamine (LSD), and/or ibogaine, and derivatives of these compounds may be useful include, but are not limited to, the following:

Major Depressive Disorder

According to the US National Institutes of Health (NIH), an estimated 17.3 million American adults had at least one depressive episode. According to the National Survey on Drug Use and Health (NSDUH), the study's definition of major depressive episode or Major Depressive Disorder is based mainly on the Diagnostic and Statistical Manual of Mental Disorders (DSM-5):
A period of at least two weeks when a person experienced a depressed mood or loss of interest or pleasure in daily activities, and had a majority of specified symptoms, such as problems with sleep, eating, energy, concentration, or self-worth.
It goes beyond normal human sadness, and leads to the inability to function normally for everyday life.
No exclusions were made for major depressive episode symptoms caused by medical illness, substance use disorders, or medication.

About 2-8% of adults with major depression die by suicide, and about 50% of people who die by suicide had depression or another mood disorder. (Richards C S, O'Hara M W (2014). The Oxford Handbook of Depression and Comorbidity. Oxford University Press. p. 254. ISBN 978-0-19-979704-2. Strakowski S, Nelson E (2015). Major Depressive Disorder. Oxford University Press. p. PT27. ISBN 978-0-19-026432-1. Bachmann, S (6 Jul. 2018). “Epidemiology of Suicide and the Psychiatric Perspective”. International Journal of Environmental Research and Public Health. 15 (7): 1425. doi:10.3390/ijerph15071425. PMC 6068947. PMID 29986446). Half of all completed suicides are related to depressive and other mood disorders.

It is estimated that the economic cost of MDD in the US is $210 Billion. This encompasses absenteeism, reduced workplace productivity, and 45-47% is healthcare costs (shared by employer/employee/society).

Alcohol Use Disorder

Approximately 17 million American suffer from Alcohol Use Disorder (AUD) with significant costs to healthcare, productivity and families. AUD (which can include Alcoholism) occurs when an individual is having difficulty controlling their drinking, being preoccupied with drinking, continues to drink even when it causes problems, drinking more to get the same effect, and withdrawal symptoms when slowing or stopping. This can also include binge drinking which is classified as having more than 5 and 4 drinks in a single session (men and women respectively). Approximately US$250 Billion is spent on healthcare, lost productivity and criminal justice every year in the US. The current treatments have limitations. Only a handful of FDA treatments, and most are poorly tolerated. Alcoholics Anonymous (AA), also has low success rates.

Opioid Use Disorder (2 Million Americans)

According to SAMHSA, approximately 11.4 million Americans misuse opioids. In addition, about 80% of Americans using heroin first started out using prescription pain relievers. The Centers for Diseases Control (CDC), estimate the all-in annual cost of opioid misuse in the US is $78.5 Billion, which includes costs of healthcare, lost productivity, treatment, and criminal justice involvement. There are medications (mainly opioid antagonists) that are used, however given the significant size of the opioid overdose health crisis, and the limited success of these treatments, there is a huge unmet need. www.samhsa.gov/data/sites/default/files/nsduh-ppt-09-2018.pdf.

Anxiety Disorders (Many Forms of Anxiety)

Are the most common mental illnesses in the US affecting approximately 40 million American adults or 18% of the population. It is estimated to cost $42 Billion to $46 Billion every year.

Pain results from noxious stimulation of nerve endings. Nociceptive pain is caused by noxious stimulation of nociceptors that transmit impulses over intact neural pathways to the spinal neurons and then to the brain. Peripheral neuropathic pain is pain due to damage of the nerve endings, mostly found in the skin, especially in the epidermis. These damaged nerve endings can generate impulses in the absence of stimulation, can be hypersensitive to normal stimulation, and/or can be triggered by remaining local inflammatory stimulation. Even a very small number of damaged and overactive small nerve fibers in the epidermis are sufficient to trigger peripheral neuropathic pain. Neuropathic pain can be debilitating and can reduce quality of life of patients considerably. This pain may persist for months or years beyond the apparent healing of any damaged tissues.

Neuropathic pain has a local inflammatory component that results in sensitization of nerve fibers. Other intact nerve fibers, such as nociceptors being present up in the stratum granulosum, innervating the same region can also be sensitized and participate in clinical symptoms of neuropathic pain (e.g., hyperalgesia). This results in a situation of local neurogenic inflammation resulting in many different clinical features such as burning, freezing, electric shock, itch, tingling, pins and needles, hyperalgesia and allodynia (pain resulting from a non-painful stimulus such as a light touch or stroke).

Peripheral nerve damage leads to enhanced transmitter release within the spinal cord and can lead to central sensitization. Increased peripheral input through primary afferents is critically involved in central sensitization and the maintenance of neuropathic pain. Peripherally acting drugs, such as lidocaine 5% medicated patches and capsaicin 8% patches, have demonstrated the ability to reduce pain in neuropathic pain syndromes. However, lidocaine patches are not easy to apply, especially on the toes and by elderly, because the patch has to be cut, and many elderly cannot reach their toes properly. Application of capsaicin creams and patches quite often induce intolerable side effects, such as an increase of burning sensation, and often the treatment has to be combined with a local anesthetic to neutralize this side effect.

In chronic pain in general, for instance, oral analgesics such as acetaminophen, nonsteroidal anti-inflammatory drugs (NSAIDs) and opioids are part of guidelines aimed at to reduce the pain. Chronic use of such oral analgesics, however, can induce serious and mortal side effects and/or detrimental drug-drug interactions.

Topical painkiller pharmaceutical compositions are also explored to help patients suffering from chronic pain. Two most commonly used topical compounds in neuropathic pain are capsaicin (vanilloid receptor agonist and counter-irritant) and lidocaine (membrane stabilizer), and both have clear drawbacks.

Clearly, there remains a pressing and long felt need in the art of developing treatment options for chronic pain in general and neuropathic pain, in particular, for the development of a novel and effective pharmaceutical composition for use in the treatment of chronic pain, the composition having reduced side effects in the patient.

Multiple Sclerosis (MS) is the most common chronic autoimmune disease of central nervous system (CNS). MS can be characterized by inflammation, demyelination and neurodegeneration, which is resulted due to invasion of autoreactive myelin-specific T lymphocytes in CNS. These T cells triggers an inflammatory response including release of proinflammatory cytokines such as tumor necrosis factor (TNF) alpha, and Interferon (INF), addition of inflammatory cells, persistent activation of macrophages resulting in oligodendrocyte death and further demyelination. MS is classified in four major forms: 1) relapsing-remitting MS (RRMS), 2) Secondary progressive MS (SPMS), 3) Primary progressive MS (PPMS) and 4) Progressive-relapsing MS (PRMS). 85% or MS patients comes under RRMS group¹⁻⁵.

It is hard to find the cure for MS due to highly heterogeneous and unpredictable course of neurological deficits. Although several immunomodulatory and immunosuppressive agents such as IFN-beta, glatiramer acetate, dimethyl fumarate, mitoxantrone, teriflunomide, cladribine, fingolimod, Siponimod and ozanimod, natalizumab, alemtuzumab, ocrelizumab, showed success in slowing disease progression and decreasing the relapse rate. The clinical efficacy and risk benefits ratio of all above drugs are very low, and the more effective drugs have a higher risk of serious adverse reactions^{6,7}.

The other process for managing MS is use of wide array of pharmacological and non-pharmacological approaches designed to minimizing disease impact while maximizing quality of life. Among pharmacological treatment for the symptomatic management of MS, cannabis and its derivatives, such as delta-9-THC and cannabidiol (CBD) are increasingly recognized as effective to treat spasticity and pain. Currently, THC:CBD (1:1) ratio-called “Sativex” marketed in more than 25 countries (except USA) for treating spasticity related to MS. Furthermore, epidemiologic studies show that MS patients increasingly use cannabis preparation for a range of symptoms associated with MS symptoms, including sleep disturbance, pain, anxiety, spasticity and even depression. According to previous research, cannabis is used by 20-60% people to treat MS and related disease conditions^{5,8,9,10}.

The current disclosure is addressed all the above drawbacks and provide patent which can have a real-world utility. Furthermore, current disclosure uses, for example, a synthetic version of cannabidiol which is manufactured in more controlled environment than the botanical source of the same. This could be another reason; synthetic version of THC/cannabidiol can provide more permeability as compared to adulterated version of it. Moreover, the current disclosure is developing transdermal matrix patches which can deliver pharmaceutical agents, such as CBD, THC, psilocybin, psilocin, lysergic acid diethylamine (LSD), and/or ibogaine, and derivatives of these compounds, or for example, synthetic cannabidiol, for 1 day, and/or 2-days, and/or 3-days, and/or 4 days, and/or 5 days, and/or 6 days, and/or 7 days, and/or up to 15 days.

The disclosure provides that the transdermal administration of pharmaceutical agents, such as CBD, THC, psilocybin, psilocin, lysergic acid diethylamine (LSD), and/or ibogaine, and derivatives of these compounds, are effective for the treatment and/or prevention of severe depression (treatment resistant), major depressive disorder, obsessive-compulsive disorder, quitting smoking, alcohol addiction, cocaine addiction, opioid addiction, anxiety (stress), adult ADHD, cluster headaches, and cancer related or other end-of-life psychological distress.

There is a need for an improved drug delivery system of pharmaceutical agents, such as CBD, THC, psilocybin, psilocin, lysergic acid diethylamine (LSD), and/or ibogaine, and derivatives of these compounds which can overcome the drawbacks associated with oral routes. Transdermal and/or topical delivery of pharmaceutical agents, such as CBD, THC, psilocybin, psilocin, lysergic acid diethylamine (LSD), and/or ibogaine, and derivatives of these compounds, the free base thereof, salts thereof, isomers thereof, amorphous forms thereof, crystalline forms thereof, co crystalline forms thereof, prodrugs thereof, analogs thereof, synthetic forms thereof, biosynthetic forms thereof, active metabolites thereof, solid solution thereof, polymorphs thereof, stereoisomers thereof, coated form thereof, ion-pairs thereof, solution thereof in solvents alone or in combinations thereof can address the challenges associated with oral drug delivery, and are useful as treatment, prevention and/or control of, for example, chronic pain or MS.

There is a need for an improved drug delivery system of psychedelics, such as CBD, THC, psilocybin, psilocin, lysergic acid diethylamine (LSD), and/or ibogaine, and derivatives of these compounds which can overcome the drawbacks associated with oral and IV routes. Transdermal delivery of pharmaceutical agents, such as CBD, THC, psilocybin, psilocin, lysergic acid diethylamine (LSD), and/or ibogaine, and derivatives of these compounds can address the challenges associated with oral and IV drug delivery. In exemplary embodiments as disclosed herein, the pharmaceutical agents, such as CBD, THC, psilocybin, psilocin, lysergic acid diethylamine (LSD), and/or ibogaine, and derivatives of these compounds, would be administered in the dosages as disclosed herein and would cause no or minimal hallucinogenic effect in a patient.

All references cited herein are incorporated herein by reference in their entireties.

### BRIEF SUMMARY

The disclosure provides compositions and methods for the treatment and/or prevention and/or control of, for example, chronic pain, using transdermal drug delivery. In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week and even up to fifteen days.

Transdermal delivery can reduce the dosing frequency of, for example, pharmaceutical agents, such as CBD, THC, psilocybin, psilocin, lysergic acid diethylamine (LSD), and/or ibogaine, and derivatives of these compounds, which is currently administered several times a day. Through transdermal delivery, transdermal compositions or transdermal formulations or transdermal patch of, for example, pharmaceutical agents, such as CBD, THC, psilocybin, psilocin, lysergic acid diethylamine (LSD), and/or ibogaine, and derivatives of these compounds, can be applied topically to skin thereby delivering the drug throughout the duration of topical application. Depending on the requirement, the duration of topical application can be once in a day, once in two days, once in three days, once in four days, once in five days, once in a week, once in fifteen days. Therefore, transdermal delivery can overcome the multiple dose regimen of oral delivery by reducing the dosing frequency.

Moreover, in transdermal drug delivery the drug is delivered slowly and continuously throughout the duration of topical application hence there are no peaks and troughs in drug plasma concentration which are associated with multiple dose administration in a day. Therefore, by transdermal delivery of, for example, pharmaceutical agents, such as CBD, THC, psilocybin, psilocin, lysergic acid diethylamine (LSD), and/or ibogaine, and derivatives of these compounds, patients can have the therapeutic effect of the drug for extended period of time without drastic changes in drug plasma concentration. The disclosure provides for continuous delivery of active agent(s) as disclosed herein.

In continuous drug delivery, active agent is continuously released and delivered to the patient to achieve systemic effect. Therefore, once applied a continuous drug delivery system can deliver active agents into systemic circulation throughout a day or even for more than one day depending on the duration of its application which can be even up to a week.

Through continuous drug delivery, for example, pharmaceutical agents, such as CBD, THC, psilocybin, psilocin, lysergic acid diethylamine (LSD), and/or ibogaine, derivatives of these compounds, or combinations thereof, can be delivered throughout the duration of application. Depending on the requirement, the duration of application can be once in a day, once in two days, once in three days, once in four days, once in five days, once in a week. Therefore, continuous drug delivery can overcome the multiple dose regimen of oral delivery by reducing the dosing frequency.

In continuous drug delivery the pharmaceutical composition comprising active agent is delivered slowly and continuously throughout the duration of administration, hence there are no peaks and troughs in drug plasma concentration of the active agent which are associated with multiple dose administration, such as oral administration, in a day. Therefore, by continuous drug delivery of, for example, pharmaceutical agents, such as CBD, THC, psilocybin, psilocin, lysergic acid diethylamine (LSD), and/or ibogaine, derivatives of these compounds, or combinations thereof, patients can have the therapeutic effect of the drug for extended period of time without drastic changes in drug plasma concentration.

In continuous drug delivery active using the systems and methods as disclosed herein active agent is delivered into systemic circulation and escapes the first pass hepatic metabolism, therefore to achieve the desired therapeutic activity less drug is required, resulting into less adverse effects or side effects. Tetrahydrocannabinol (THC) and Cannabidiol (CBD) have high lipid solubility and after oral administration undergoes hepatic first pass metabolism, therefore of the administered dose only 10%-20% reaches systemic circulation, thus as compared to oral dose, a continuous drug delivery a small dose of tetrahydrocannabinol (THC), cannabidiol (CBD), or combinations thereof can give the desired therapeutic effects at a lower dose than oral.

Furthermore, using a continuous drug delivery system and methods as disclosed herein is easy, noninvasive and convenient. Administration of a drug compound by continuous drug does not require medical supervision as patients can control the administration themselves.

In certain embodiments, the continuous drug delivery system and methods as disclosed herein may comprise, for example, a transdermal patch, or other transdermal formulation. In certain embodiments, the continuous drug delivery system and methods as disclosed herein may comprise a pump device, such as ones which are commonly used to deliver one or more fluids to a targeted individual. For example, a medical infusion pump device may be used to deliver a medicine to a patient as part of a medical treatment. The active agent(s) that is delivered by the infusion pump device can depend on the condition of the patient and the desired treatment plan. For example, infusion pump devices have been used to deliver insulin into the subcutaneous tissue and to the vasculature of diabetes patients to regulate blood-glucose levels. In some circumstances, the dosage of medicine delivered by the infusion pump can be calculated by the infusion pump system. In these circumstances, the infusion pump system can take into account many variables, including user input, when making such calculations.

Other forms of continuous drug delivery systems and methods may make use of fluid delivery to a targeted individual. For example, insulin, glucagon, or another medicine can be injected using a manual syringe or a single use injection “pen.” In some circumstances, an injectable form of glucagon is used in emergency aid of severe hypoglycemia when the victim is unconscious or for other reasons cannot take glucose orally. The glucagon fluid can be rapidly injected to the patient by intramuscular, intravenous, or subcutaneous injection, and quickly raises the blood glucose level of the patient.

One category of devices for delivering such fluids is that of pumps that have been developed for the administration of insulin and other medicaments for those suffering from both type I and type II diabetes. Some pumps configured as portable infusion devices can provide Continuous drug for the treatment of diabetes. Such therapy may include, e.g., the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. patent application Ser. No. 13/838,617, U.S. patent application Ser. No. 13/827,707 and U.S. Pat. No. 8,287,495, each of which is hereby incorporated herein by reference in its entirety.

In certain embodiments as disclosed herein continuous drug delivery system may comprise, for example, a patch pump, or micro pump. Patch pumps are small pumps, typically ambulatory, that are carried directly on the skin under the user's clothing. Such a pump generally is situated directly on the injection site such that no tubing is required to deliver the insulin or other medicament to the patient. Patch pumps typically are at least in part disposable, meant to be worn for a day or two and then discarded for a new patch pump.

Furthermore, transdermal delivery is easy, noninvasive and convenient. Administration of a transdermal patch or transdermal composition does not require medical supervision as patients can topically apply the transdermal patch or transdermal composition themselves. Therefore, transdermal delivery can overcome the drawbacks of injections which are often painful and requires medical supervision.

With respect to pharmaceutical agents, such as CBD, THC, psilocybin, psilocin, lysergic acid diethylamine (LSD), and/or ibogaine, and derivatives of these compounds, it is expected that interpatient variability in pharmacologic response will be less with transdermal delivery as drug plasma concentration can be controlled by controlling the rate of drug delivery from transdermal composition or transdermal patch. With transdermal delivery a small amount of CBD and/or THC can be delivered for longer duration than oral administration. Transdermal formulations, for example, of pharmaceutical agents, such as CBD, THC, psilocybin, psilocin, lysergic acid diethylamine (LSD), and/or ibogaine, and derivatives of these compounds, also provide more abuse deterrence than immediate release dosage forms. Moreover, in case of any adverse effect, side effect or emergency transdermal delivery gives the liberty to terminate the therapy anytime by taking off the transdermal patch or transdermal composition from skin.

As per above stated reasons for the treatment and/or prevention and/or control of chronic pain, transdermal delivery can provide patient friendly, simplified and convenient therapeutic regimen over traditional delivery systems. Transdermal delivery can reduce the dosing frequency of pharmaceutical agents, such as CBD, THC, psilocybin, psilocin, lysergic acid diethylamine (LSD), and/or ibogaine, and derivatives of these compounds, Depending on the necessity, dosing frequency can be once in a day, once in two days, once in three days, once in four days, once in five days, once in six days, once in a week, once in ten days. Through transdermal administration of drug combination, two or more drugs can be delivered simultaneously. Depending on the necessity, dosing frequency of transdermal patch or transdermal composition containing drug combination can be once in a day, once in two days, once in three days, once in four days, once in five days, once in six days, once in a week once in ten days. It would be a great addition to the patient compliance.

The disclosure provides a transdermal and/or topical pharmaceutical composition comprising: at least one active agent selected from the group consisting of: about 0.1% to about 50% of an active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, naturally derived forms thereof, active metabolites thereof, polymorph thereof, solid solution thereof, coated form thereof, and combinations thereof, further wherein the pharmaceutical composition comprises: about 10% to about 99.9% of an adhesive and/or polymer; optionally, about 0.1% to about 99% of a permeation enhancer; optionally, about 0.1% to about 99% of a solvent, wherein said pharmaceutical composition will have no or minimal hallucinogenic or psychoactive effect in a patient to whom the pharmaceutical composition is applied.

The disclosure provides a transdermal and/or topical pharmaceutical composition wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL.

The disclosure provides a transdermal and/or topical pharmaceutical composition wherein the pharmaceutical formulation provides a dose of active agent to a patient equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, 500 microgram/day, about 0.001 mg/day, 0.01 mg/day, 0.025 mg/day. 0.05 mg/day, 0.1 mg/day, 0.25 mg/day, 0.5 mg/day, 1 mg/day, 10 mg/day, or 25 mg/day.

The disclosure provides a transdermal and/or topical pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, naturally derived forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

The disclosure provides a transdermal and/or topical pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

The disclosure provides a transdermal and/or topical pharmaceutical composition comprising one or more active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for transdermal delivery.

The disclosure provides a transdermal and/or topical pharmaceutical composition comprising one or more active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for topical delivery.

The disclosure provides a transdermal and/or topical pharmaceutical composition wherein said CBD, THC, psilocybin, psilocin, lysergic acid diethylamine (LSD), and/or ibogaine, the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof, is produced by a natural route.

The disclosure provides a transdermal and/or topical pharmaceutical composition wherein said tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof, is produced by a synthetic route.

The disclosure provides a transdermal and/or topical pharmaceutical composition formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, a multilayer transdermal matrix system, or transdermal drug-in-adhesive matrix formulation.

The disclosure provides a transdermal and/or topical pharmaceutical composition formulated as a topical liquid formulation, topical semisolid formulation, topical gel formulation, topical polymer matrix formulation, topical adhesive matrix formulation, topical film forming gel formulation, or topical film forming spray formulation.

The disclosure provides a transdermal and/or topical pharmaceutical composition which is formulated as a patch. The disclosure provides a transdermal and/or topical pharmaceutical composition which is formulated as two or more patches. The disclosure provides a transdermal and/or topical pharmaceutical composition wherein the two or more patches each compromise the same active agent. The disclosure provides a transdermal and/or topical pharmaceutical composition wherein the two or more patches each comprise different active agents. The disclosure provides a transdermal and/or topical pharmaceutical composition wherein the two or more patches each comprise the same or different active agents.

The disclosure provides a transdermal and/or topical pharmaceutical composition which is formulated as a transdermal patch.

The disclosure provides a transdermal and/or topical pharmaceutical composition formulated as a transdermal patch, wherein the transdermal patch is selected from the group consisting of a reservoir patch, a microreservoir patch, a micro-dosing patch, a matrix patch, a drug in adhesive patch, a pressure sensitive adhesive patch, extended-release transdermal film a liquid reservoir system, a microreservoir patch, a mucoadhesive patch, multilayer transdermal matrix system, and combinations thereof.

The disclosure provides a transdermal and/or topical pharmaceutical composition which is formulated as a topical patch.

The disclosure provides a transdermal and/or topical pharmaceutical composition formulated as a topical patch, wherein the topical patch is selected from the group consisting of a reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, multilayer transdermal matrix system, and combinations thereof.

The disclosure provides a transdermal and/or topical pharmaceutical composition which is formulated as metered dose transdermal gel, metered dose transdermal spray, a film forming gel, a film forming spray, or a meter-dose aerosol.

The disclosure provides a transdermal and/or topical pharmaceutical composition formulated as microneedles.

The disclosure provides a transdermal and/or topical pharmaceutical composition formulated as a liquid formulation, transdermal semisolid formulation, or transdermal polymer matrix formulation, transdermal adhesive matrix formulation, film forming gel formulation, film forming spray formulation.

The disclosure provides a transdermal and/or topical pharmaceutical composition further comprising at least one additional active agent selected from the group consisting of THC, CBD, psilocybin, psilocin, lysergic acid diethylamine (LSD), and/or ibogaine, antidepressant drug, NSAIDS, anticonvulsants drug, corticosteroid drug, pain relievers, lidocaine, menthol, capsaicin, methyl salicylate, lidocaine, capsaicin, Tricyclic Antidepressants, amitriptyline, imipramine, nortriptyline, desipramine, doxepin, SNRIs and SSRIs, duloxetine, venlafaxine, fluoxetine, milnacipran, diclofenac, aspirin, naproxen, ibuprofen, ketoprofen, celecoxib, meloxicam, acetaminophen, cox-2 inhibitors, celecoxib, anticonvulsants, carbamazepine, gabapentin, lamotrigine, pregabalin, oxcarbazepine, lamotrigine, valproic acid, menthol, camphor, methyl salicylate, salicylates, corticosteroid drugs, triamcinolone, methylprednisolone, cortisone, prednisone, dexamethasone, opioids, and combinations thereof.

The disclosure provides a transdermal and/or topical pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

The disclosure provides a transdermal and/or topical pharmaceutical composition wherein the adhesive is selected from the group consisting of pressure sensitive adhesives, silicone polymers, bio psa 4302, bio-psa 4202, acrylic pressure sensitive adhesives, duro-tak 87-2156, duro-tak 387-2287, duro-tak 87-9301, duro-tak 387-2051, polyisobutylene, polyisobutylene low molecular weight, polyisobutylene medium molecular weight, polyisobutylene 35000 mw, acrylic copolymers, rubber based adhesives, hot melt adhesives, styrene-butadiene copolymers, bentonite, all water and/or organic solvent swellable polymers and combinations thereof.

The disclosure provides a transdermal and/or topical pharmaceutical composition wherein said polymer is present and is selected from the group consisting of natural polymers, polysaccharides. agar, alginic acid and derivatives, Cassia tora, collagen, gelatin, gellum gum, guar gum, pectin, potassium cargeenan, sodium carageenan, tragacanth, xantham, gum copal, chitosan, resin, semisynthetic polymers, cellulose, methylcellulose, ethyl cellulose, carboxymethyl cellulose, hydroxylpropyl cellulose, hydroxylpropylmethyl cellulose, synthetic polymers, carboxyvinyl polymers, carbomers, carbopol 940, carbopol 934, carbopol 9′71p NF, polyethylene, clays, silicates, bentonite, silicon dioxide, polyvinyl alcohol, acrylic polymers (eudragit), acrylic acid esters, polyacrylate copolymers, polyacrylamide, polyvinyl pyrrolidone homopolymer, polyvinyl pyrrolidone copolymers, PVP, Kollidon 30, poloxamer, isobutylene, ethyl vinyl acetate copolymers, natural rubber, synthetic rubber, and combinations thereof.

The disclosure provides a transdermal and/or topical pharmaceutical composition wherein said permeation enhancer is present, and is selected from the group consisting of dimethylsulfoxide, dimethylacetamide, dimethylformamide, decymethylsulfoxide, dimethylisosorbide, azone, pyrrolidones, N-methyl-2-pyrrolidone, 2-pyrrolidon, esters, fatty acid esters, propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate, fatty acids, capric acid, caprylic acid, lauric acid, oleic acid, myristic acid, linoleic acid, stearic acid, palmitic acid, alcohols, fatty alcohols, glycols, oleyl alcohol, nathanol, dodecanol, propylene glycol, glycerol, ethers, alcohol, diethylene glycol monoethyl ether, urea, triglycerides, triacetin, polyoxyethylene fatty alcohol ethers, polyoxyethylene fatty acid esters, esters of fatty alcohols, essential oils, surfactant type enhancers, brij, sodium lauryl sulfate, tween, polysorbate, terpene, terpenoids, and combinations thereof.

The disclosure provides a transdermal and/or topical pharmaceutical composition wherein said solvent is present, and is selected from the group consisting of methanol, ethanol, isopropyl alcohol, butanol, propanol, polyhydric alcohols, glycols, propylene glycol, polyethylene glycol, dipropylene glycol, hexylene glycol, butylene glycol, glycerine, derivative of glycols, pyrrolidone, N methyl 2-pyrrolidone, 2 pyrrolidone, sulfoxides, dimethyl sulfoxide, decymethylsulfoxide, dimethylisosorbide, mineral oils, vegetable oils, sesame oil water, polar solvents, semi polar solvents, non polar solvents, volatile chemicals, ethanol, propanol, ethyl acetate, acetone, methanol, dichloromethane, chloroform, toluene, IPA, hexane, acids, acetic acid, lactic acid, levulinic acid, bases, pentane, dimethylformamide, butane, lipids, and combinations thereof.

The disclosure provides a transdermal and/or topical pharmaceutical composition which is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

The disclosure provides a transdermal and/or topical pharmaceutical composition which is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

The disclosure provides a transdermal and/or topical pharmaceutical composition co-administered with at least one additional an active agent selected from the group consisting of: medications administered for treatment and/or management and/or prevention and/or control of symptoms associated with neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

The disclosure provides a transdermal and/or topical pharmaceutical composition indicated for the treatment and/or prevention and/or control of chronic pain, multiple sclerosis, severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient.

The disclosure provides a transdermal and/or topical pharmaceutical composition wherein the pharmaceutical composition provides a continuous, sustained delivery of the pharmaceutical composition to mitigate peak and valley pharmacokinetic behavior of the active agent. The disclosure provides a transdermal and/or topical pharmaceutical composition wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a transdermal and/or topical pharmaceutical composition wherein the pharmaceutical composition provides a continuous, sustained delivery of the pharmaceutical composition via intravenous or subcutaneous infusion.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient comprising: selecting a patient in need of treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress; topically applying the pharmaceutical composition as disclosed herein, thereby treating and/or preventing and/or controlling severe depression (treatment resistant), major depressive disorder, obsessive-compulsive disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in said patient, wherein said patient experiences no or minimal psychoactive or hallucinogenic effects from said transdermal pharmaceutical composition.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient wherein the topical application of a pharmaceutical composition for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient, wherein the transdermal patch is applied at a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient wherein the topical application of a pharmaceutical composition for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient wherein the pharmaceutical composition is applied to the patient separately, sequentially, or simultaneously.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient further providing a constant rate of delivery of the active components of the transdermal patch over a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient further providing a steady absorption rates of the active components of the transdermal patch over a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient further achieving a constant blood serum levels of the active components of the transdermal patch over a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient further achieving a reduced variability in dosage of the active components of the transdermal patches over a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient further providing a plasma concentration of the active components of the transdermal patch in a therapeutic range over a time period selected from the group consisting of once in a day, once in two days, once in three days, once in four days, once in five days, once in six days, once in a week, once in ten days, and once in fifteen days.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient further providing a plasma concentration of the active components of the transdermal patch in a therapeutic range of about 0.01 ng/mL to about 500 ng/mL.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient wherein the pharmaceutical composition provides a continuous, sustained delivery of the pharmaceutical composition to mitigate peak and valley pharmacokinetic behavior of the active agent.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient wherein the pharmaceutical composition provides provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

The disclosure provides a method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, and cancer related or other end-of-life psychological distress in a patient wherein the pharmaceutical composition provides a continuous, sustained delivery of the pharmaceutical composition via intravenous or subcutaneous infusion.

The disclosure provides a method for the treatment and/or prevention and/or control of chronic pain in a patient comprising: selecting a patient in need of treatment and/or prevention and/or control of chronic pain; topically applying a pharmaceutical composition as disclosed herein, thereby treating, preventing and/or controlling chronic pain in the patient, wherein said patent experiences no or minimal psychoactive or hallucinogenic effects from said transdermal pharmaceutical composition.

The disclosure provides a method for the treatment and/or prevention and/or control of chronic pain in a patient wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

The disclosure provides a method for the treatment and/or prevention and/or control of chronic pain in a patient wherein the topical application of a pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days.

The disclosure provides a method for the treatment and/or prevention and/or control of chronic pain in a patient wherein the pharmaceutical compositions are applied to the patient separately, sequentially or simultaneously. The disclosure provides a method for the treatment and/or prevention and/or control of chronic pain in a patient wherein the pharmaceutical composition provides a continuous, sustained delivery of the pharmaceutical composition to mitigate peak and valley pharmacokinetic behavior of the active agent. The disclosure provides a method for the treatment and/or prevention and/or control of chronic pain in a patient wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a method for the treatment and/or prevention and/or control of chronic pain in a patient wherein the pharmaceutical composition provides a continuous, sustained delivery of the pharmaceutical composition via intravenous or subcutaneous infusion.

The disclosure provides a method for the treatment and/or prevention and/or control of adjustment disorder in a patient comprising: selecting a patient in need of treatment and/or prevention and/or control of adjustment disorder; topically applying the pharmaceutical composition as disclosed herein, thereby treating and/or preventing and/or controlling adjustment disorder in the patient, wherein said patent experiences no or minimal psychoactive or hallucinogenic effects from said transdermal pharmaceutical composition. The disclosure provides a method for the treatment and/or prevention and/or control of adjustment disorder in a patient wherein the topical application of a pharmaceutical composition for the treatment and/or prevention and/or control of adjustment disorder, wherein the transdermal patch is applied at a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient further providing a constant rate of delivery of the active components of the transdermal patch over a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient further providing a steady absorption rates of the active components of the transdermal patch over a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient further achieving a constant blood serum levels of the active components of the transdermal patch over a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient further achieving a reduced variability in dosage of the active components of the transdermal patches over a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient further providing a plasma concentration of the active components of the transdermal patch in a therapeutic range over a time period selected from the group consisting of once in a day, once in two days, once in three days, once in four days, once in five days, once in six days, once in a week, once in ten days, and once in fifteen days. The disclosure provides a method for the treatment and/or prevention and/or control of adjustment disorder in a patient wherein the pharmaceutical compositions are applied to the patient separately, sequentially, or simultaneously. The disclosure provides a method for the treatment and/or prevention and/or control of adjustment disorder in a patient further providing a plasma concentration of the active components of the transdermal patch in a therapeutic range of about 0.01 ng/mL to about 500 ng/mL. The disclosure provides a method for the treatment and/or prevention and/or control of adjustment disorder in a patient wherein the pharmaceutical composition provides a continuous, sustained delivery of the pharmaceutical composition to mitigate peak and valley pharmacokinetic behavior of the active agent. The disclosure provides a method for the treatment and/or prevention and/or control of adjustment disorder in a patient wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a method for the treatment and/or prevention and/or control of adjustment disorder in a patient wherein the pharmaceutical composition provides a continuous, sustained delivery of the pharmaceutical composition via intravenous or subcutaneous infusion.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject. The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

It must be noted that as used herein and in the appended claims, the singular forms “a”, “an”, and “the” include plural referents unless the context clearly dictates otherwise. Thus, for example, reference to “a compound” includes a plurality of compounds.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this disclosure belongs.

As used herein the following terms have the following meanings.

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies. The term “treatment” as used herein has its conventional meaning and is here to be considered in its broadest context. The term “treatment” is intended to encompass topical and/or transdermal administration of active compounds, i.e., active pharmaceutical ingredients e.g., in a pharmaceutical composition, according to the disclosure, with the aim to alleviate an undesired condition, and therapeutic administration to eliminate or reduce the extent or symptoms of the condition. Treatment does not necessarily imply that a subject is treated until total recovery.

As used herein, the term “about” when used in conjunction with a stated numerical value or range has the meaning reasonably ascribed to it by a person skilled in the art, i.e., denoting somewhat more or somewhat less than the stated value or range.

The terms transdermal and topical are used interchangeably

The term “chronic pain” or “chronic pain states” as used herein, is defined as any pain lasting longer than, for example, about 6 to about 12 weeks.

The term “neuropathic pain” as used herein has its conventional meaning and here means a pain arising as a direct or indirect consequence of a lesion or disease affecting the somatosensory system (central and/or peripheral). Neuropathic pain as used herein, includes all types of neuropathic pain, such as peripheral neuropathy caused by diabetes type 1 or 2, induced by various noxious substances such as alcohol, due to various deficiencies such as vitamin B1, B6 and/or B12 deficiency, various intoxications, such as hypervitaminosis B6, hypothyroidism, chemotherapeutic compound (e.g., paclitaxel or other taxane derivative, vincristine or other vinca alkaloids, cisplatin or other platinum derivate), drug-induced neuropathy, compounds for the treatment of infectious diseases (e.g., streptomycin, didanosine or zalcitabine), or any other chemically toxic compound. Other peripheral neuropathies include the following: trigeminal neuralgia, post-herpetic neuralgia, intercostal neuralgia, entrapment neuropathy (e.g., carpal tunnel syndrome, tarsal tunnel syndrome, abdominal cutaneous nerve entrapment syndrome), small fiber neuropathy, hereditary motor and sensory neuropathies, chronic inflammatory demyelinating polyneuropathy, sciatic pain chronic idiopathic sensory neuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and other mono- and polyneuropathies. Furthermore, under the term “neuropathic pain” also the following is included: complex regional pain syndrome type I and II (reflex sympathetic dystrophy), central neuropathic pain (e.g., thalamic neuropathy, spinal cord injury neuropathy, post stroke pain, multiple sclerosis neuropathy, syringomyelia, spinal cord tumors), phantom limb pain, restless genital syndrome (pain), post-surgical scar pain including cardiac surgery and mastectomy.

The term “inflammatory pain” as used herein has its conventional meaning and here means a pain that arises from inflammation that may be caused but by not limited to trauma, burns, extreme cold, fractures, (osteo)arthritis, rheumatoid arthritis, chronic strains, surgery, infection and autoimmune diseases excessive stretching, infections and vasoconstriction. Multiple inflammatory mediators can directly affect nociceptors or may sensitize them to touch or movement, even some distance from the inflammatory field.

The term “musculoskeletal pain” as used herein has its conventional meaning and here means a pain that affects the muscles, ligaments, tendons, bones, joints and/or soft tissues that are part of the musculoskeletal system. Musculoskeletal pain as used herein, includes all types of pain due to damage of muscle tissue as a result of wear and tear of daily activities. Trauma to an area (jerking movements, auto accidents, falls, sport injuries, fractures, sprains, strains dislocations, and direct blows to the muscle) also can cause musculoskeletal pain. Other causes of musculoskeletal pain include postural strain, repetitive movements, overuse, and prolonged immobilization, misuse of muscles, fibromyalgia, lumbar pain, pain due to increased muscle tone, and tendinitis due to overuse.

The term “analgesic” or “analgesics” as used herein has its conventional meaning and here refers to compounds, agents, drugs or substances that reduce pain in its broadest context.

The term “co-analgesic” or “co-analgesics” as used herein has its conventional meaning and here refers to compounds, agents, drugs or substances whose primary indication is for a purpose other than pain relief, which compounds demonstrate analgesic activity.

The term “reinstating analgesic effects” as used herein has its regular scientific meaning and is here referring to the capability (of a compound or of a composition) of reinstating an analgesic effect of at least one analgesic compound or at least one co-analgesic compound, when decreasing analgesic effect occurs after repeated use of a topical formulation containing at least one analgesic or co-analgesic compound.

The term “effect booster” or “co-analgesic effect booster” or “therapeutic effect booster” or “booster effect” or “synergistic effect” as used herein has its conventional meaning and here means the enhancement of a therapeutic effect induced by a co-analgesic compound (“co-analgesic”) leading to 1) intensified therapeutic effects of an active pharmaceutical ingredient with the purpose of alleviating neuropathic pain, inflammatory pain, musculoskeletal pain, pain due to muscle spasms, and/or other chronic pain states, 2) a faster onset of pain relieving effect, 3) a longer duration of analgesia, and/or 4) reinstating analgesic effects, when decreasing analgesic effect occurs after repeated use of a topical pharmaceutical composition containing at least one analgesic compound (“analgesic”) or co-analgesic compound.

The term “topical formulation” as used herein has its conventional meaning and here refers to a formulation that may be applied to skin or mucosa with the aim that a therapeutically active compound penetrates in and/or through the skin, e.g., a topical pharmaceutical composition of the disclosure, e.g., a pharmaceutical composition provided as a topical cream.

As used herein, the term “transdermal delivery” means delivery of drug into systemic circulation through the skin.

As used herein, the term “topical delivery” means delivery of drug to skin for local effect

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. Disclosure provides for, for example, topical and/or transdermal formulations comprising one or more of the following active agents: CBD, THC, psilocybin, psilocin, lysergic acid diethylamine (LSD), and/or ibogaine, and derivatives of these compounds.

### Psilocybin

Psilocybin is a naturally occurring psychedelic compound produced by more than 200 species of mushrooms, collectively known as psilocybin mushrooms. The most potent are members of the genus Psilocybe, such as P. azurescens, P. semilanceata, and P. cyanescens, but psilocybin has also been isolated from about a dozen other genera.

Once ingested, psilocybin is rapidly metabolized to psilocin, which then acts on serotonin receptors in the brain. The mind-altering effects of psilocybin typically last from two to six hours, although to individuals under the influence of psilocybin, the effects may seem to last much longer, since the drug can distort the perception of time. Psilocybin has a low toxicity and a relatively low harm potential, and reports of lethal doses of the drug are rare. Several modern bioanalytical methods have been adapted to rapidly and accurately screen the levels of psilocybin in mushroom samples and body fluids. Since the 1990s, there has been a renewal of scientific research into the potential medical and psychological therapeutic benefits of psilocybin for treating conditions including obsessive-compulsive disorder (OCD), cluster headaches, and anxiety related to terminal cancer.

Psilocybin is also referred to as [3-(2-dimethylaminoethyl)-1H-indol-4-yl] dihydrogen phosphate, and given the CAS number 520-52-5.

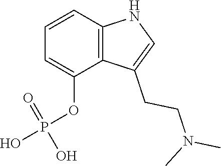 [SMILES: CN(C)CCc1c[nH]c2cccc(OP(=O)(O)O)c12]

Psilocin (also known as 4-HO-DMT, psilocine, psilocyn, or psilotsin) is a substituted tryptamine alkaloid and a serotonergic psychedelic substance. It is present in most psychedelic mushrooms together with its phosphorylated counterpart psilocybin.

Psilocin also referred to as 4-hydroxy-N,N-dimethyltryptamine, and given the CAS number 520-53-6.

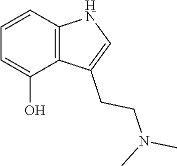

LSD: (+)-Lysergic acid diethylamide, 9,10-didehydro-N,N-diethyl-6-methylergoline-8β-carboxamide, known as LSD, is a hallucinogen which acts on the central nervous system and alters sensory perception. A concentration of from 20 to 80 μg of LSD is sufficient to induce hallucination (Nelson, C. and Foltz, R. Anal. Chem, 64, 1578-1585, 1992).

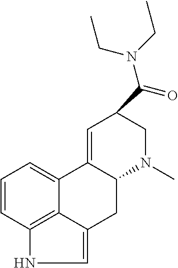 [SMILES: CCN(CC)C(=O)[C@@H]1C=C2c3cccc4[nH]cc(c34)C[C@H]2N(C)C1]

Ibogaine has been used as a botanical preparation from the root bark of Iboga tabernathe for over 100 years both as a crude preparation and as semisynthetic ibogaine, which was marketed in France until about 1970. High doses of ibogaine have been suggested to be useful as a treatment for pain and other conditions. However, the use of such high doses of ibogaine is associated with hallucinations and other negative side effects. In the United States, ibogaine is classified as a Schedule I controlled substance.

While ibogaine has been disclosed for treatment of substance addiction, its use in humans is complicated by the fact that the ranges in the prior art are exceptionally broad (0.01 to 1000 mg/kg body weight). Furthermore, human clinical studies demonstrate that the lower dosing of ibogaine has minimal impact on the alleviation of pain in patients. Thus, the previously disclosed broad range has now been found to be insufficient for human therapy at the lower end of this range.

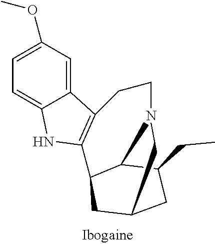 [SMILES: CC[C@H]1C[C@@H]2C[C@H]3c4[nH]c5ccc(OC)cc5c4CCN(C2)[C@@H]13]

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. The disclosure provides for, for example, transdermal formulations and/or topical formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton Chemical structure is shown below as formula I

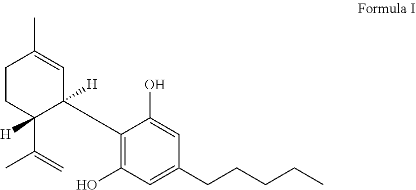 [SMILES: C=C(C)[C@@H]1CCC(C)=C[C@H]1Cc1c(O)cc(CCCCC)cc1O]

Tetrahydrocannbinol (THC) IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.
Chemical structure is shown below as formula II

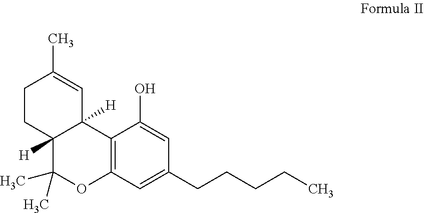 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, etc. alone or in combinations thereof.

As used herein, synthetic cannabinoids include at least the following: AM-087 is an analgesic drug that is a cannabinoid agonist derivative of A8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor a (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1. and has been shown to have immunomodulatory effects, and may be useful in the treatment of pain and inflammation; JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051— an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB1, JWH-176—1-([(1E)-3-pentylinden-1-ylidine]methyl)naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-ylethyl)indol-3-yl)-4-methylnaphthalen-1-ylmethane; JWH-193—(1-(2-morpholin-4-ylethyl)indol-3-yl)-4-methylnaphthalen ylmethanone; JWH-194—2-methyl-1-pentyl-1H-indol-3-yl-(4-methyl-1-naphthyl)methane; JWH-195—(1-(2-morpholin-4-ylethyl)indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl) methoxynaphthalen-1-ylmethanone; JWH-199—(1-(2-morpholin-4-ylethyl)indol-3-yl) methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl)indole); JWH-258—a potent and mildly selective CB1 agonist; JWH-302— (1-pentyl-3-(3-methoxyphenylacetyl)indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl)indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal and/or topical compositions as disclosed herein comprising, for example, biosynthetic CBD, alone or in combination with other active agents.

According to certain embodiments, transdermal and/or topical compositions described herein are for the prevention and/or treatment of pain and/or inflammation. According to certain embodiments, transdermal and/or topical compositions described herein are for the reduction in severity of pain and/or inflammation.

According to certain embodiments described herein, pharmaceutical composition or transdermal formulation or topical formulation contains cannabidiol and/or THC-the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, coated form thereof, solution of cannabidiol in methanol, alone or in combinations thereof. More preferably transdermal and topical formulation may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, active metabolites thereof, polymorphs thereof, stereoisomers thereof, coated form thereof, solution of cannabidiol in methanol, alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites polymorphs thereof, stereoisomers thereof, coated form thereof, ion-pairs thereof. For example, the active agent's free base or its salts or its isomers or its amorphous form or its crystalline form or its co crystalline form or its solid solution or its prodrugs or its analogs or its derivatives or synthetic forms polymorphs thereof, ion-pairs thereof, stereoisomers thereof, coated form thereof. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this disclosure are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term “active agent” includes, for example, CBD, THC, and other cannabinoids as disclosed herein, as well as psychedelics, such as psilocybin, psilocin, lysergic acid diethylamine (LSD), and/or ibogaine, and derivatives of these compounds, and the free base thereof, salts thereof, isomers thereof, amorphous forms thereof, polymorphs forms thereof, coated forms thereof, crystalline forms thereof, ion par forms thereof, co crystalline forms thereof, prodrugs thereof, analogs thereof, derivatives thereof, stereoisomers forms thereof, synthetic forms thereof, alone or in combinations thereof. In certain embodiments the active agent is highly purified. In certain embodiments the active agent is a highly pure synthetic. In certain embodiments the active agent is a highly purified extract of active agent which comprises at least 90%, 91%, 92%, 93%, 94%, 95%, 96%, 97%, 98%, 99%, 99.5%, or 99.75% (w/w).

In certain embodiments the active agent is provided at a concentration of at least 90%, 91%, 92%, 93%, 94%, 95%, 96%, 97%, 98%, 99%, 99.5%, 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, or 45 mg/kg/day. In certain embodiments, the dose of active agent is equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, or 500 microgram/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg.

In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% of the formulation. In exemplary embodiments, formulations of the disclosure may comprise active agent at a concentration of, for example, about 0.1 to about 50%, about 0.1 to about 20%, about 1 to 20%, of about 5% to 25%, about 10% to about 20%, about 15% to about 20%, about 15% to about 18%, about 30% to about 70%, about 35% to about 65%, about 63.13%, about 40% to about 64%, about 95 to about 98%, or about 95 to about 97%, w/w. In exemplary formulations of the disclosure, the active agent will represent approximately about 0.1 wt % to about 50% wt %, 1 wt % to 75 wt %, preferably 2 wt % to 30 wt %, more preferably 10 wt. % to 20 wt. % of the formulation. In certain embodiments as disclosed herein the active agents as disclosed herein may be microdosed. Microdosing is born from the “set and setting” school of psychedelic therapy and one of its intellectual progeny, James Fadiman. The Stanford-trained Fadiman has worked with psychedelics for decades and runs a kind of cottage industry around espousing their powers. In his 2011 book The Psychedelic Explorer's Guide and at a conference talk that same year, Fadiman laid out the concept of microdosing. To microdose, one was to take a dose roughly 1/10th of a trip-inducing dose (10 micrograms of LSD) every three or four days and go about their daily life. Most of what's known about the benefits of microdosing comes from self-reports Fadiman collected (and continues to collect) where microdosers described how the practice transformed their lives. In them, microdosers speak of anxiety and depression melting away, and feelings of determination and self-resolve that helped them achieve professional success. Some color-blind men even saw color for the first time.”

As disclosed herein, the term “microdose” refers to a non-psychoactive or non-hallucinogenic dose of a pharmaceutical active agent as disclosed herein. In exemplary embodiments, a microdose of the active agent cannabinoids such as CBD, THC, or other cannabinoids, or psychedelics, such as psilocybin, psilocin, lysergic acid diethylamine (LSD), and/or ibogaine, and derivatives of these compounds may, for example, a dose roughly 1/10th of a psychedelic, high-, or trip-inducing dose (“macrodose”). For example, 10 micrograms of LSD can be considered a microdose. In exemplary embodiments, these dosages would be administered to a patient, for example, every two, three, four, five, six, or seven days.

### Additional Active Agents

As used herein the term “combination administration” of a compound, therapeutic agent or known drug with the combination of the present disclosure means administration of the drug and the one or more compounds at such time that both the known drug and/or combination will have a therapeutic effect. In some cases, this therapeutic effect will be synergistic. Such concomitant administration can involve concurrent (i.e., at the same time), prior, or subsequent administration of the drug with respect to the administration of the composition and/or combination of the present disclosure. A person of ordinary skill in the art would have no difficulty determining the appropriate timing, sequence and dosages of administration for particular drugs of the present disclosure.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; anti diarrheal; antidiuretic; anti-emetic; anti-epileptic; antifibrinolytic; antifungal; anti hemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; anti seborrheic; anti secretory; anti spasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid supressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine A1 antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

Examples of active ingredients comprises, but is not limited to any of the following, for example, alone or in combination: 16-alpha fluorocstradiol, 16alpha-gitoxin, 16-epiestriol, 17 alpha dihydroequilenin, 17 alpha estradiol, 17 beta estradiol, 17 hydroxy progesterone, 1alpha-hydroxyvitamin D2, 1-dodecpyrrolidinone, 20-epi-1,25 dihydroxyvitamin D3, 22-oxacalcitriol, 2CVV, 2′-nor-cGMP, 3-isobutyl GABA, 5-ethynyluracil, 6-FUDCA, 7-methoxytacrine, Abamectin, abanoquil, abecarnil, abiraterone, Ablukast, Ablukast Sodium, Acadesine, acamprosate, Acarbose, Acebutolol, Acecainide Hydrochloride, Aceclidine, aceclofenae, Acedapsone, Aceglutamide Aluminum, Acemannan, Acetaminophen, Acetazolamide, Acetohexamide, Acetohydroxamic Acid, acetomepregenol, Acetophenazine Maleate, Acetosulfone Sodium, Acetylcholine Chloride, Acetylcysteine, acetyl-L-carnitine, acetylmethadol, Acifran, acipimox, acitemate, Acitretin, Acivicin, Aclarubicin, aclatonium, Acodazole Hydrochloride, aconiazide, Acrisorcin, Acrivastine, Acronine, Actisomide, Actodigin, Acyclovir, acylfulvene, adafenoxate, adapalene, Adapalene, adatanserin, Adatanserin Hydrochloride, adecypenol, adecypenol, Adefovir, adelmidrol, ademetionine, Adenosine, Adinazolam, Adipheinine Hydrochloride, adiposin, Adozelesin, adrafinil, Adrenalone, airbutamine, alacepril, Alamecin, Alanine, Alaproclate, alaptide, Albendazole, albolabrin, Albuterol, Albutoin, Alclofenae, Alclometasone Dipropionate, Alcloxa, aldecalmycin, Aldesleukin, Aldioxa, Alendronate Sodium, alendroni c acid, alentemol, Alentemol Hydrobromide, Aletamine Hydrochloride, Aleuronium Chloride, Alexidine, alfacalcidol, Alfentanil Hydrochloride, alfuzosin, Algestone Acetonide, alglucerase, Aliflurane, alinastine, Alipamide, Allantoin, Allobarbital, Allopurinol, ALL-TK antagonists, Alonimid, alosetron, Alosetron Hydrochloride, Alovudine, Alpertine, Alpha Amylase, alpha idosone, Alpidem, Alprazolam, Alprenolol Hydrochloride, Alprenoxime Hydrochloride, Alprostadil, Alrestatin Sodium, Altanserin Tartrate, Alteplase, Althiazide, Altretamine, altromycin B, Alverinc Citrate, Alvircept Sudotox, Amadinone Acetate, Amantadine Hydrochloride, ambamustine, Ambomycin, Ambruticin, Ambuphylline, Ambuside, Amcinafal, Amcinonide, Amdinocillin, Amdinocillin Pivoxil, Amedalin Hydrochloride, amelometasone, Ameltolide, Amesergide, Ametantrone Acetate, amezinium metilsulfate, amfebutamone, Amfenac Sodium, Amflutizole, Amicycline, Amidephrine Mesylate, amidox, Amifloxacin, amifostine, Amikacin, Amiloride Hydrochloride, Aminacrine Hydrochloride, Aminobenzoate Potassium, Aminobenzoate Sodium, Aminocaproic Acid, Aminoglutethimide, Aminohippurate Sodium, aminolevulinic acid, Aminophylline, A minorex, Aminosalicylate sodium, Aminosalicylic acid, Amiodarone, Amiprilose Hydrochloride, Amiquinsin Hydrochloride, amisulpride, Amitraz, Amitriptyline Hydrochloride, Amlexanox, amlodipine, Amobarbital Sodium, Amodiaquine, Amodiaquine Hydrochloride, Amorolfine, Amoxapine, Amoxicillin, Amphecloral, Amphetamine Sulfate, Amphomycin, Amphotericin B, Ampicillin, ampiroxicam, Ampyzine Sulfate, Amquinate, Amrinone, amrinone, amrubicin, Amsacrine, amylin, amythiamicin, Anagestone Acetate, anagrelide, Anakinra, ananain, anaritide, Anaritide Acetate, Anastrozole, Anazolene Sodium, Ancrod, andrographolide, Androstenedione, angiogenesis inhibitors, Angiotensin Amide, Anidoxime, Anileridine, Anilopam Hydrochloride, Aniracetam, Anirolac, Anisotropine Methylbromide, Anistreplase, Anitrazafen, anordrin, antagonist D, antagonist G, antarelix, Antazoline Phosphate, Anthelmycin, Anthralin, Anthramycin, antiandrogen, Acedapsone, Felbamate, antiestrogen, antineoplaston, Antipyrine, antisense oligonucleotides, apadoline, apafant, Apalcillin Sodium, apaxifylline, Apazone, aphidicolin glycinate, Apixifylline, Apomorphine Hydrochloride, apraclonidine, Apraclonidine Hydrochloride, Apramycin, Aprindine, Aprindine Hydrochloride, aprosulate sodium, Aprotinin, Aptazapine Maleate, aptiganel, apurinic acid, apurinic acid, aranidipine, Aranotin, Arbaprostil, arbekicin, arbidol, Arbutamine Hydrochloride, Arclofenin, Ardeparin Sodium, argatroban, Arginine, Argipressin Tannate, Arildone, aripiprazol, arotinolol, Arpinocid, Arteflene, Artilide Fumarate, asimadoline, aspalatone, Asparaginase, Aspartic Acid, Aspartocin, asperfuran, Aspirin, aspoxicillin, Asprelin, Astemizole, Astromicin Sulfate, asulacrine, atamestane, Atenolol, atevirdine, Atipamezole, Atiprosin Maleate, Atolide, Atorvastatin Calcium, Atosiban, Atovaquone, atpenin B, Atracurium Besylate, atrimustine, atrinositol, Atropine, Auranofin, aureobasidin A, Aurothioglucose, Avilamycin, Avoparcin, Avridine, Axid, axinastatin 1, axinastatin 2, axinastatin 3, Azabon, Azacitidinie, Azaclorzine Hydrochloride, Azaconazole, azadirachtine, Azalanstat Dihydrochloride, Azaloxan Fumarate, Azanator Maleate, Azanidazole, Azaperone, Azaribine, Azaserine, azasetron, Azatadine Maleate, Azathioprine, Azathioprine Sodium, azatoxin, azatyrosine, azelaic acid, azelastine, azelnidipine, Azepindole, Azetepa, azimilide, Azithromycin, Azlocillin, Azolimine, Azosemide, Azotomycin, Aztreonam, Azumolene Sodium, Bacampicillin Hydrochloride, baccatin III, Bacitracin, Baclofen, bacoside A, bacoside B, bactobolamine, balanol, balazipone, balhimycin, balofloxacin, balsalazide, Bambermycins, bambuterol, Bamethan Sulfate, Bamifylline Hydrochloride, Bamidazole, baohuoside 1, Barmastine, barnidipine, Basifungin, Batanopride Hydrochloride, batebulast, Batelapine Maleate, Batimastat, beauvericin, Becanthone Hydrochloride, becaplermin, becliconazole, Beclomethasone Dipropionate, befloxatone, Beinserazide, Belfosdil, Belladonna, Beloxamide, Bemesetron, Bemitradine, Bemoradan, Benapryzine Hydrochloride, Benazepril Hydrochloride, Benazeprilat, Bendacalol Mesylate, Bendazac, Bendroflumethiazide, benflumetol, benidipine, Benorterone, Benoxaprofen, Benoxaprofen, Benoxinate Hydrochloride, Benperidol, Bentazepam, Bentiromide, Benurestat, Benzbromarone, Benzethonium Chloride, Benzetimide Hydrochloride, Benzilonium Bromide, Benzindopyrine Hydrochloride, benzisoxazole, Benzocaine, benzochlorins, Benzoctamine Hydrochloride, Benzodepa, benzoidazoxan, Benzonatate, Benzoyl Peroxide, Benzoylpas Calcium, benzoylstaurosporine, Benzquinamide, Benzthiazide, benztropine, Benztropine Mesylate, Benzydamine Hydrochloride, Benzylpenicilloyl Polylysine, bepridil, Bepridil Hydrochloride, Beractant, Beraprost, Berefrine, berlafenone, bertosamil, B erythromycin, besipirdine, beta-alethine, betaclamycin B, Betamethasone, betamipron, betaxolol, Betaxolol Hydrochloride, Bethanechol Chloride, Bethanidine Sulfate, betulinic acid, bevantolol, Bevantolol Hydrochloride, Bezafibrate, bFGF inhibitor, Bialamicol Hydrochloride, Biapenem, Bicalutamide, Bicifadine Hydrochloride, Biclodil Hydrochloride, Bidisomide, bifemelane, Bifonazole, bimakalim, bimithil, Bindarit, Biniramycin, binospirone, bioxalomycin alpha2, Bipenamol Hydrochloride, Biperiden, Biphenamine Hydrochloride, biriperone, bisantrene, bisaramil, bisaziridinylspermine, bi s-benzimidazole A, bis-benzimidazole B, bisnafide, Bisobrin Lactate, Bisoprolol, Bispyrithione Magsulfex, bistramide D, bistramide K, bistratene A, Bithionolate Sodium, Bitolterol Mesylate, Bivalirudin, Bizelesin, Bleomycin Sulfate, Bolandiol Dipropionate, Bolasterone, Boldenone Undecylenate, boldine, Bolenol, Bolmantalate, bopindolol, Bosentan, Boxidine, brefeldin, breflate, Brequinar Sodium, Bretazenil, Bretylium Tosylate, Brifentanil Hydrochloride, brimonidine, Brinolase, Brocresine, Brocrinat, Brofoxine, Bromadoline Maleate, Bromazepam, Bromchlorenone, Bromelains, bromfenac, Brominidione, Bromocriptine, Bromodiphenhydramine Hydrochloride, Bromoxanide, Bromperidol, Bromperidol Decanoate, Brompheniramine Maleate, Broperamole, Bropirimine, Brotizolam, Bucainide Maleate, bucindolol, Buclizine Hydrochloride, Bucromarone, Budesonide, budipine, budotitane, Buformin, Bumetamide, Bunaprolast, bunazosin, Bunolol Hydrochloride, Bupicomide, Bupivacaine Hydrochloride, Buprenorphine Hydrochloride, Bupropion Hydrochloride, Buramate, Buserelin Acetate, Buspirone Hydrochloride, Busulfan, Butabarbital, Butacetin, Butaclamol Hydrochloride, Butalbital, Butamben, Butamirate Citrate, Butaperazine, Butaprost, Butedronate Tetrasodium, butenafine, Buterizine, buthionine sulfoximine, Butikacin, Butilfenin, Butirosin Sulfate, Butixirate, butixocort propionate, Butoconazole Nitrate, Butonate, Butopamine, Butoprozine Hydrochloride, Butorphanol, Butoxamine Hydrochloride, Butriptyline Hydrochloride, Cactinomycin, Cadexomer Iodine, Caffeine, calanolide A, Calcifediol, Calcipotriene, calcipotriol, Calcitonin, Calcitriol, Calcium Undecylenate, calphostin C, Calusterone, Cambendazole, camonagrel, camptothecin derivatives, canarypox IL-2, candesartan, Candicidin, candoxatril, candoxatrilat, Caniglibose, Canrenoate Potassium, Canrenone, capecitabine, Capobenate Sodium, Capobenic Acid, Capreomycin Sulfate, capromab, capsaicin, Captopril, Capuride, Caracemide, Carbachol, Carbadox, Carbamazepine, Carbamide Peroxide, Carbantel Lauryl Sulfate, Carbaspirin Calcium, Carbazeran, carbazomycin C, Carbenicillin Potassium, Carbenoxolone Sodium, Carbetimer, carbetocin, Carbidopa, Carbidopa-Levodopa, Carbinoxamine Maleate, Carbiphene Hydrochloride, Carbocloral, Carbocysteine, Carbol-Fuchsin, Carboplatin, Carboprost, carbovir, carboxamide-amino-triazo-le, carboxyamidotriazole, carboxymethylated beta-1,3-glucan, Carbuterol Hydrochloride, CaRest M3, Carfentanil Citrate, Carisoprodol, Carmantadine, Carmustine, CARN 700, Camidazole, Caroxazone, carperitide, Carphenazine Maleate, Carprofen, Carsatrin Succinate, Cartazolate, carteolol, Carteolol Hydrochloride, cartilage derived inhibitor, Carubicin Hydrochloride, Carumonam Sodium, carvedilol, carvotroline, Carvotroline Hydrochloride, carzelesin, casein kinase inhibitors (ICOS), castanospermine, caurumonam, cebaracetam, cecropin B, Cedefingol, Cefaclor, Cefadroxil, Cefamandole, Cefaparole, Cefatrizine, Cefazaflur Sodium, Cefazolin, Cefbuperazone, cefcapene pivoxil, cefdaloxime pentexil tosilate, Cefdinir, cefditoren pivoxil, Cefepime, cefetamet, Cefetecol, cefixime, cefluprenam, Cefinenoxime Hydrochloride, Cefinetazole, cefminlox, cefodizime, Cefonicid Sodium, Cefoperazone Sodium, Ceforamide, cefoselis, Cefotaxime Sodium, Cefotetan, cefotiam, Cefoxitin, cefozopran, cefpimizole, Cefpiramide, cefpirome, cefpodoxime proxetil, cefprozil, Cefroxadine, cefsulodin, Ceftazidime, cefteram, ceftibuten, Ceftizoxime Sodium, ceftriaxone, Cefuroxime, celastrol, celikalim, celiprolol, cepacidiine A, Cephacetrile Sodium, Cephalexin, Cephaloglycin, Cephaloridine, Cephalothin Sodium, Cephapirin Sodium, Cephradine, cericlamine, cerivastatin, Ceronapril, certoparin sodium, Ceruletide, Cetaben Sodium, Cetalkonium Chloride, Cetamolol Hydrochloride, cetiedil, cetirizine, Cetophenicol, Cetraxate Hydrochloride, cetrorelix, Cetylpyridinium Chloride, Chenodiol, Chlophedianol Hydrochloride, Chloral Betaine, Chlorambucil, Chloramphenicol, Chlordantoin, Chlordiazepoxide, Chlorhexidine Gluconate, chlorins, Chlormadinone Acetate, chloroorienticin A, Chloroprocaine Hydrochloride, Chloropropamide, Chloroquine, chloroquinoxaline sulfonamide, Chlorothiazide, Chlorotrianisene, Chloroxine, Chloroxylenol, Chlorphenesin Carbamate, Chlorpheniramine Maleate, Chlorpromazine, Chlorpropamide, Chlorprothixene, Chlortetracycline Bisulfate, Chlorthalidone, Chlorzoxazone, Cholestyramine Resin, Chromonar Hydrochloride, cibenzoline, cicaprost, Ciclafrine Hydrochloride, Ciclazindol, ciclesonide, cicletanine, Ciclopirox, Cicloprofen, cicloprolol, Cidofovir, Cidoxepin Hydrochloride, Cifenline, Ciglitazone, Ciladopa Hydrochloride, cilansetron, Cilastatin Sodium, Cilazapril, cilnidipine, Cilobamine Mesylate, cilobradine, Cilofungin, cilostazol, Cimaterol, Cimetidine, cimetropium bromide, Cinalukast, Cinanserin Hydrochloride, Cinepazet Maleate, Cinflumide, Cingestol, cinitapride, Cinnamedrine, Cinnarizine, cinolazepam, Cinoxacin, Cinperene, Cinromide, Cintazone, Cintriamide, Cioteronel, Cipamfylline, Ciprefadol Succinate, Ciprocinonide, Ciprofibrate, Ciprofloxacin, ciprostene, Ciramadol, Cirolemycin, cisapride, cisatracurium besilate, Cisconazole, Cisplatin, cis-porphyrin, cistinexine, citalopram, Citenamide, citicoline, citreamicin alpha, cladribine, Clamoxyquin Hydrochloride, Clarithromycin, clausenamide, Clavulanate Potassium, Clazolam, Clazolimine, clebopride, Clemastine, Clentiazem Maleate, Clidinium Bromide, clinafloxacin, Clindamycin, Clioquinol, Clioxanide, Cliprofen, clobazam, Clobetasol Propionate, Clobetasone Butyrate, Clocortolone Acetate, Clodanolene, Clodazon Hydrochloride, clodronic acid, Clofazimine, Clofibrate, Clofilium Phosphate, Clogestone Acetate, Clomacran Phosphate, Clomegestone Acetate, Clometherone, clomethiazole, clomifene analogues, Clominorex, Clomiphene, Clomipramine Hydrochloride, Clonazepam, Clonidine, Clonitrate, Clonixeril, Clonixin, Clopamide, Clopenthixol, Cloperidone Hydrochloride, clopidogrel, Clopimozide, Clopipazan Mesylate, Clopirac, Cloprednol, Cloprostenol Sodium, Clorazepate Dipotassium, Clorethate, Clorexolone, Cloroperone Hydrochloride, Clorprenaline Hydrochloride, Clorsulon, Clortermine Hydrochloride, Closantel, Closiramine Aceturate, Clothiapine, Clothixamide Maleate Cloticasone Propionate, Clotrimazole, Cloxacillin Benzathine, Cloxyquin, Clozapine, Cocaine, Coccidioidin, Codeine, Codoxime, Colchicine, colestimide, Colestipol Hydrochloride, Colestolone, Colforsin, Colfosceril Palmitate, Colistimethate Sodium, Colistin Sulfate, collismycin A, collismycin B, Colterol Mesylate, combretastatin A4, combretastatin analogue, complestatin, conagenin, Conorphone Hydrochloride, contignasterol, contortrostatin, Cormethasone Acetate, Corticorelin Ovine Triflutate, Corticotropin, Cortisone Acetate, Cortivazol, Cortodoxone, cosalane, costatolide, Cosyntropin, cotinine, Coumadin, Coumermycin, crambescidin 816, Crilvastatin, crisnatol, Cromitrile Sodium, Cromolyn Sodium, Crotamiton, cryptophycin 8, cucumariosid, Cuprimyxin, curacin A, curdlan sulfate, curiosin, Cyclacillin, Cyclazocine, cyclazosin, cyclic HPMPC, Cyclindole, Cycliramine Maleate, Cyclizine, Cyclobendazole, cyclobenzaprine, cyclobut A, cyclobut G, cyclocapron, Cycloguanil Pamoate, Cycloheximide, cyclopentanthraquinones, Cyclopenthiazide, Cyclopentolate Hydrochloride, Cyclophenazine Hydrochloride, Cyclophosphamide, cycloplatam, Cyclopropane, Cycloserine, cyclosin, Cyclosporine, cyclothialidine, Cyclothiazide, cyclothiazomycin, Cyheptamide, cypemycin, Cypenamine Hydrochloride, Cyprazepam, Cyproheptadine Hydrochloride, Cyprolidol Hydrochloride, cyproterone, Cyproximide, Cysteamine, Cysteine Hydrochloride, Cystine, Cytarabine, Cytarabine Hydrochloride, cytarabine ocfosfate, cytochalasin B, cytolytic factor, cytostatin, Dacarbazine, dacliximab, dactimicin, Dactinomycin, daidzein, Daledalin Tosylate, dalfopristin, Dalteparin Sodium, Daltroban, Dalvastatin, danaparoid, Danazol, Dantrolene, daphlnodorin A, dapiprazole, dapitant, Dapoxetine Hydrochloride, Dapsone, Daptomycin, Darglitazone Sodium, darifenacin, darlucin A, Darodipine, darsidomine, Daunorubicin Hydrochloride, Dazadrol Maleate, Dazepinil Hydrochloride, Dazmegrel, Dazopride Fumarate, Dazoxiben Hydrochloride, Debrisoquin Sulfate, Decitabine, deferiprone, deflazacort, Dehydrocholic Acid, dehydrodidemnin B, Dehydroepiandrosterone, delapril, Delapril Hydrochloride, Delavirdine Mesylate, delequamine, delfaprazine, Delmadinone Acetate, delmopinol, delphinidin, Demecarium Bromide, Demeclocycline, Demecycline, Demoxepam, Denofungin, deoxypyridinoline, Depakote, deprodone, Deprostil, depsidomycin, deramciclane, dermatan sulfate, Desciclovir, Descinolone Acetonide, Desflurane, Desipramine Hydrochloride, desirudin, Deslanoside, deslorelin, desmopressin, desogestrel, Desonide, Desoximetasone, desoxoamiodarone, Desoxycorticosterone Acetate, detajmium bitartrate, Deterenol Hydrochloride, Detirelix Acetate, Devazepide, Dexamethasone, Dexami sole, Dexbrompheniramine Maleate, Dexchlorpheniramine Maleate, Dexclamol Hydrochloride, Dexetimide, Dexfenfluramine Hydrochloride, dexifosfamide, Deximafen, Dexivacaine, dexketoprofen, dexloxiglumide, Dexmedetomidine, Dexormaplatin, Dexoxadrol Hydrochloride, Dexpanthenol, Dexpemedolac, Dexpropranolol Hydrochloride, Dexrazoxane, dexsotalol, dextrin 2-sulphate, Dextroamphetamine, Dextromethorphan, Dextrorphan Hydrochloride, Dextrothyroxine Sodium, dexverapamil, Dezaguanine, dezinamide, dezocine, Diacetolol Hydrochloride, Diamocaine Cyclamate, Diapamide, Diatrizoate Meglumine, Diatrizoic Acid, Diaveridine, Diazepam, Diaziquone, Diazoxide, Dibenzepin Hydrochloride, Dibenzothiophene, Dibucaine, Dichliorvos, Dichloralphenazone, Dichlorphenamide, Dicirenone, Diclofenac Sodium, Dicloxacillin, dicranin, Dicumarol, Dicyclomine Hydrochloride, Didanosine, didemnin B, didox, Dienestrol, dienogest, Diethylcarbamazine Citrate, diethylhomospermine, diethylnorspermine, Diethylpropion Hydrochloride, Diethylstilbestrol, Difenoximide Hydrochloride, Difenoxin, Diflorasone Diacetate, Difloxacin Hydrochloride, Difluanine Hydrochloride, Diflucortolone, Diflumidone Sodium, Diflunisal, Difluprednate, Diftalone, Digitalis, Digitoxin, Digoxin, Dihexyverine Hydrochloride, dihydrexidine, dihydro-5-azacytidine, Dihydrocodeine Bitartrate, Dihydroergotamine Mesylate, Dihydroestosterone, Dihydrostreptomycin Sulfate, Dihydrotachysterol, dihydrotaxol, 9-, Dilantin, Dilevalol Hydrochloride, Diltiazem Hydrochloride, Dimefadane, Dimefline Hydrochloride, Dimenhydrinate, Dimercaprol, Dimethadione, Dimethindene Maleate, Dimethisterone, dimethyl prostaglandin A1, Dimethyl Sulfoxide, dimethylhomospermine, dimiracetam, Dimoxamine Hydrochloride, Dinoprost, Dinoprostone, Dioxadrol Hydrochloride, dioxamycin, Diphenhydramine Citrate, Diphenidol, Diphenoxylate Hydrochloride, diphenyl spiromustine, Dipivefin Hydrochloride, Dipivefrin, dipliencyprone, diprafenone, dipropylnorspermine, Dipyridamole, Dipyrithione, Dipyrone, dirithromycin, discodermolide, Disobutamide, Disofenin, Disopyramide, Disoxaril, disulfiram, Ditekiren, Divalproex Sodium, Dizocilpine Maleate, Dobutamine, docarpamine, Docebenone, Docetaxel, Doconazole, docosanol, dofetilide, dolasetron, Ebastine, ebiratide, ebrotidine, ebselen, ecabapide, ecabet, ecadotril, ecdisteron, echicetin, echistatin, Echothiophate Iodide, Eclanamine Maleate, Eclazolast, ecomustine, Econazole, ecteinascidin 722, edaravone, Edatrexate, edelfosine, Edifolone Acetate, edobacomab, Edoxudine, edrecolomab, Edrophonium Chloride, edroxyprogesteone Acetate, efegatran, eflornithine, efonidipine, egualcen, Elantrine, eleatonin, elemene, eletriptan, elgodipine, eliprodil, Elsamitrucin, eltenae, Elucaine, emalkalim, emedastine, Emetine Hydrochloride, emiglitate, Emilium Tosylate, emitefur, emoctakin, Enadoline Hydrochloride, enalapril, Enalaprilat, Enalkiren, enazadrem, Encyprate, Endralazine Mesylate, Endrysone, Enflurane, englitazone, Enilconazole, Enisoprost, Enlimomab, Enloplatin, Enofelast, Enolicam Sodium, Enoxacin, enoxacin, enoxaparin sodium, Enoxaparin Sodium, Enoximone, Enpiroline Phosphate, Enprofylline, Enpromate, entacapone, enterostatin, Enviradene, Enviroxime, Ephedrine, Epicillin, Epimestrol, Epinephrine, Epinephryl Borate, Epipropidine, Epirizole, epirubicin, Epitetracycline Hydrochloride, Epithiazide, Epoetin Alfa, Epoetin Beta, Epoprostenol, Epoprostenol Sodium, epoxymexrenone, epristeride, Eprosartan, eptastigmine, equilenin, Equilin, Erbulozole, erdosteine, Ergoloid Mesylates, Ergonovine Maleate, Ergotamine Tartrate, ersentilide, Ersofermin, erythritol, Erythrityl Tetranitrate, Erythromycin, Esmolol Hydrochloride, Esorubicin Hydrochloride, Esproquin Hydrochloride, Estazolam, Estradiol, Estramustine, estramustine analogue, Estrazinol Hydrobromide, Estriol, Estrofurate, estrogen agonists, estrogen antagonists, Estrogens, Conjugated, Estrogens, Esterified, Estrone, Estropipate, esuprone, Etafedrine Hydrochloride, Etanidazole, etanterol, Etarotene, Etazolate Hydrochloride, Eterobarb, ethacizin, Ethacrynate Sodium, Ethacrynic Acid, Ethambutol Hydrochloride, Ethamivan, Ethanolamine Oleate, Ethehlorvynol, Ether, Ethinyl estradiol, Ethiodized Oil, Ethionamide, Ethonam Nitrate, Ethopropazine Hydrochloride, Ethosuximide, Ethotoin, Ethoxazene Hydrochloride, Ethybenztropine, Ethyl Chloride, Ethyl Dibunate, Ethylestrenol, Ethyndiol, Ethynerone, Ethynodiol Diacetate, Etibendazole, Etidocaine, Etidronate Disodium, Etidronic Acid, Etifenin, Etintidine Hydrochloride, etizolam, Etodolac, Etofenamate, Etoformin Hydrochloride, Etomidate, Etonogestrel, Etoperidone Hydrochloride, Etoposide, Etoprine, Etoxadrol Hydrochloride, Etozolin, etrabamine, Etretinate, Etryptamine Acetate, Eucatropine Hydrochloride, Eugenol, Euprocin Hydrochloride, eveminomicin, Exametazime, examorelin, Exaprolol Hydrochloride, exemestane, fadrozole, faeriefungin, Famciclovir, Famotidine, Fampridine, fantofarone, Fantridone Hydrochloride, faropenem, fasidotril, fasudil, fazarabine, fedotozine, felbamate, Felbinac, Felodipine, Felypressin, Fenalamide, Fenamole, Fenbendazole, Fenbufen, Fencibutirol, Fenclofenac, Fenclonine, Fenclorac, Fendosal, Fenestrel, Fenethylline Hydrochloride, Fenfluramine Hydrochloride, Fengabine, Fenimide, Fenisorex, Fenmetozole Hydrochloride, Fenmetramide, Fenobam, Fenoctimine Sulfate, fenofibrate, fenoldopam, Fenoprofen, Fenoterol, Fenpipalone, Fenprinast Hydrochloride, Fenprostalene, Fenquizone, fenretinide, fenspiride, Fentanyl Citrate, Fentiazac, Fenticlor, fenticonazole, Fenyripol Hydrochloride, fepradinol, ferpifosate sodium, ferristene, ferrixan, Ferrous Sulfate, Dried, Ferumoxides, ferumoxsil, Fetoxylate Hydrochloride, fexofenadine, Fezolamine Fumarate, Fiacitabine, Fialuridine, Fibrinogen 1 125, filgrastim, Filipin, finasteride, Flavodilol Maleate, flavopiridol, Flavoxate Hydrochloride, Flazalone, flecainide, flerobuterol, Fleroxacin, flesinoxan, Flestolol Sulfate, Fletazepam, flezelastine, flobufen, Floctafenine, flomoxef, Flordipine, florfenicol, florifenine, flosatidil, Flosequinan, Floxacillin, Floxuridine, fluasterone, Fluazacort, Flubanilate Hydrochloride, Flubendazole, Flucindole, Flucloronide, Fluconazole, Flucytosine, Fludalanine, Fludarabine Phosphate, Fludazonium Chloride, Fludeoxyglucose F 18, Fludorex, Fludrocortisone Acetate, Flufenamic Acid, Flufenisal, Flumazenil, flumecinol, Flumequine, Flumeridone, Flumethasone, Flumetramide, Flumezapine, Fluminorex, Flumizole, Flumoxonide, flunarizine, Flunidazole, Flunisolide, Flunitrazepam, Flunixin, fluocalcitriol, Fluocinolone Acetonide, Fluocinonide, Fluocortin Butyl, Fluocortolone, Fluorescein, fluorodaunorunicin hydrochloride, Fluorodopa F 18, Fluorometholone, Fluorouracil, Fluotracen Hydrochloride, Fluoxetine, Fluoxymesterone, fluparoxan, Fluperamide, Fluperolone Acetate, Fluphenazine Decanoate, flupirtine, Fluprednisolone, Fluproquazone, Fluprostenol Sodium, Fluquazone, Fluradoline Hydrochloride, Flurandrenolide, Flurazepam Hydrochloride, Flurbiprofen, Fluretofen, flurithromycin, Flurocitabine, Flurofamide, Flurogestone Acetate, Flurothyl, Fluroxene, Fluspiperone, Fluspirilene, Fluticasone Propionate, flutrimazole, Flutroline, fluvastatin, Fluvastatin Sodium, fluvoxamine, Fluzinamide, Folic Acid, Follicle regulatory protein, Folliculostatin, Fomepizole, Fonazine Mesylate, forasartan, forfenimex, forfenirmex, formestane, Formocortal, formoterol, Fosarilate, Fosazepam, Foscarnet Sodium, fosfomycin, Fosfonet Sodium, fosinopril, Fosinoprilat, fosphenyloin, Fosquidone, Fostedil, fostriecin, fotemustine, Fuchsin, Basic, Fumoxicillin, Fungimycin, Furaprofen, Furazolidone, Furazolium Chloride, Furegrelate Sodium, Furobufen, Furodazole, Furosemide, Fusidate Sodium, Fusidic Acid, gabapentin, Gadobenate Dimeglumine, gadobenic acid, gadobutrol, Gadodiamide, gadolinium texaphyrin, Gadopentetate Dimegiumine, gadoteric acid, Gadoteridol, Gadoversetamide, galantamine, galdansetron, Galdansetron Hydrochloride, Gallamine Triethiodide, gallium nitrate, gallopamil, galocitabine, Gamfexine, gamolenic acid, Ganciclovir, ganirelix, gelatinase inhibitors, Gemcadiol, Gemcitabine, Gemeprost, Gemfibrozil, Gentamicin Sulfate, Gentian Violet, gepirone, Gestaclone, Gestodene, Gestonorone Caproate, Gestrinone, Gevotroline Hydrochloride, girisopam, glaspimod, glaucocalyxin A, Glemanserin, Gliamilide, Glibornuride, Glicetanile Sodium, Gliflumide, Glimepiride, Glipizide, Gloximonam, Glucagon, glutapyrone, glutathione inhibitors, Glutethimide, Glyburide, glycopine, glycopril, Glycopyrrolate, Glyhexamide, Glymidine Sodium, Glyoctamide, Glyparamide, Gold Au 198, Gonadoctrinins, Gonadorelin, Gonadotropins, Goserelin, Gramicidin, Granisetron, grepafloxacin, Griseofulvin, Guaiapate, Guaithylline, Guanabenz, Guanabenz Acetate, Guanadrel Sulfate, Guancydine, Guanethidine Monosulfate, Guanfacine Hydrochloride, Guanisoquin Sulfate, Guanoclor Sulfate, Guanoctine Hydrochloride, Guanoxabenz, Guanoxan Sulfate, Guanoxyfen Sulfate, Gusperimus Trihydrochloride, Halazepam, Halcinonide, halichondrin B, Halobetasol Propionate, halofantrine, Halofantrine Hydrochloride, Halofenate, Halofuginone Hydrobromide, halomon, Halopemide, Haloperidol, halopredone, Haloprogesterone, Haloprogin, Halothane, Halquinols, Hamycin, Han memopausal gonadotropins, hatomamicin, hatomarubigin A, hatomarubigin B, hatomarubigin C, hatomarubigin D, Heparin Sodium, hepsulfam, heregulin, Hetacillin, Heteronium Bromide, Hexachlorophene:Hydrogen Peroxide, Hexafluorenium Bromide, hexamethylene bisacetamide, Hexedine, Hexobendine, Hexoprenaline Sulfate, Hexylresorcinol, Histamine Phosphate, Histidine, Histoplasmin, Histrelin, Homatropine Hydrobromide, Hoquizil Hydrochloride, Human chorionic gonadotropin, Hycanthone, Hydralazine Hydrochloride, Hydralazine Polistirex, Hydrochlorothiazide, Hydrocodone Bitartrate, Hydrocortisone, Hydroflumethiazide, Hydromorphone Hydrochloride, Hydroxyamphetamine Hydrobromide, Hydroxychloroquine Sulfate, Hydroxyphenamate, Hydroxyprogesterone Caproate, Hydroxyurca, Hydroxyzine Hydrochloride, Hymecromone, Hyoscyamine, hypericin, Ibafloxacin, ibandronic acid, ibogaine, Ibopamine, ibudilast, Ibufenac, Ibuprofen, Ibutilide Fumarate, Icatibant Acetate, Ichthammol, Icotidine, idarubicin, idoxifene, Idoxuridine, idramantone, Iemefloxacin, Iesopitron, Ifetroban, Ifosfamide, Ilepeimide, illimaquinone, ilmofosine, ilomastat, Ilonidap, iloperidone, iloprost, Imafen Hydrochloride, Imazodan Hydrochloride, imidapril, imidazenil, imidazoacridones, Imidecyl Iodine, Imidocarb Hydrochloride, Imidoline Hydrochloride, Imidurea, Imiloxan Hydrochloride, Imipenem, Imipramine Hydrochloride, imiquimod, immunostimulant peptides, Impromidine Hydrochloride, Indacrinone, Indapamide, Indecainide Hydrochloride, Indeloxazine Hydrochloride, Indigotindisulfonate Sodium, indinavir, Indocyanine Green, Indolapril Hydrochloride, Indolidan, indometacin, Indomethacin Sodium, Indoprofen, indoramin, Indorenate Hydrochloride, Indoxole, Indriline Hydrochloride, inocoterone, inogatran, inolimomab, Inositol Niacinate, Insulin, interferons, interleukins, Intrazole, Intriptyline Hydrochloride, iobenguane, Iobenzamic Acid, iobitridol, Iocarmate Meglumine, Iocarmic Acid, Iocetamic Acid, Iodamide, Iodine, Iodipamide Meglumine, Iodixanol, iodoamiloride, Iodoantipyrine I 131, Iodocholesterol I 131, iododoxorubicin, Iodohippurate Sodium I 131, Iodopyracet I 125, Iodoquinol, Iodoxamate Meglumine, Iodoxamie Acid, Ioglicic Acid, Iofetamine Hydrochloride I 123, iofratol, Ioglucol, Ioglucomide, Ioglycamic Acid, Iogulamide, Iohexol, iomeprol, Iomethin I 125, Iopamidol, Iopanoic Acid, iopentol, lophendylate, Ioprocemic Acid, iopromide, Iopronic Acid, Iopydol, Iopydone, iopyrol, Iosefamic Acid, Ioseric Acid, Iosulamide Meglumine, Iosumetic Acid, Iotasul, Iotetric Acid, Iothalamate Sodium, Iothalamic Acid, iotri side, Iotrolan, Iotroxic Acid, Iotyrosine 1 131, Ioversol, Ioxagiate Sodium, Ioxaglate Meglumine, Ioxaglic Acid, ioxilan, Ioxotrizoic Acid, ipazilide, ipenoxazone, ipidacrine, Ipodate Calcium, ipomeanol, 4-, Ipratropium Bromide, ipriflavone, Iprindole, Iprofenin, Ipronidazole, Iproplatin, Iproxamine Hydrochloride, ipsapirone, irbesartan, irinotecan, irloxacin, iroplact, irsogladine, Irtemazole, isalsteine, Isamoxole, isbogrel, Isepamicin, isobengazole, Isobutamben, Isocarboxazid, Isoconazole, Isoetharine, isofloxythepin, Isoflupredone Acetate, Isoflurane, Isoflurophate, isohomohalicondrin B, Isoleucine, Isomazole Hydrochloride, Isomylamine Hydrochloride, Isoniazid, Isopropamide Iodide, Isopropyl Alcohol, isopropyl unoprostone, Isoproterenol Hydrochloride, Isosorbide, Isosorbide Mononitrate, Isotiquimide, Isotretinoin, Isoxepac, Isoxicam, Isoxsuprine Hydrochloride, isradipine, itameline, itasetron, Itazigrel, itopride, Itraconazole, Ivermectin, jasplakinolide, Josamycin, kahalalide F, Kalafungin, Kanamycin Sulfate, Ketamine Hydrochloride, Ketanserin, Ketazocine, Ketazolam, Kethoxal, Ketipramine Fumarate, Ketoconazole, Ketoprofen, Ketorfanol, ketorolac, Ketotifen Fumarate, Kitasamycin, Labetalol Hydrochloride, Lacidipine, lacidipine, lactitol, lactivicin, laennec, lafutidine, lamellarin-N triacetate, lamifiban, Lamivudine, Lamotrigine, lanoconazole, Lanoxin, lanperisone, lanreotide, Lansoprazole, latanoprost, lateritin, laurocapram, Lauryl Isoquinolinium Bromide, Lavoltidine Succinate, lazabemide, Lecimibide, leinamycin, lemildipine, leminoprazole, lenercept, Leniquinsin, lenograstim, Lenperone, lentinan sulfate, leptin, leptolstatin, lercanidipine, Lergotrile, lerisetron, Letimide Hydrochloride, letrazuril, letrozole, Leucine, leucomyzin, Leuprolide Acetate, leuprolide+estrogen+progesterone, leuprorelin, Levamfetamine Succinate, levamisole, Levdobutamine Lactobionate, Leveromakalim, levetiracetam, Leveycloserine, levobetaxolol, levobunolol, levobupivacaine, levocabastine, levocarnitine, Levodopa, levodropropizine, levofloxacin, Levofuraltadone, Levoleucovorin Calcium, Levomethadyl Acetate, Levomethadyl Acetate Hydrochloride, levomoprolol, Levonantradol Hydrochloride, Levonordefrin, Levonorgestrel, Levopropoxyphene Napsylate, Levopropylcillin Potassium, levormeloxifene, Levorphanol Tartrate, levosimendan, levosulpiride, Levothyroxine Sodium, Levoxadrol Hydrochloride, Lexipafant, Lexithromycin, liarozole, Libenzapril, Lidamidine Hydrochloride, Lidocaine, Lidofenin, Lidoflazine, Lifarizine, Lifibrate, Lifibrol, Linarotene, Lincomycin, linear polyamine analogue, Linogliride, Linopirdine, linotroban, linsidomine, lintitript, lintopride, Liothyronine I 125, liothyronine sodium, Liotrix, lirexapride, lisinopril, lissoclinamide 7, Lixazinone Sulfate, lobaplatin, Lobenzarit Sodium, Lobucavir, Lodelaben, Iodoxamide, Lofemizole Hydrochloride, Lofentanil Oxalate, Lofepramine Hydrochloride, Lofexidine Hydrochloride, lombricine, Lomefloxacin, lomerizine, Lometraline Hydrochloride, lometrexol, Lomofungin, Lomoxicam, Lomustine, Lonapalene, lonazolac, lonidamine, Loperamide Hydrochloride, loracarbef, Loraj mine Hydrochloride, loratadine, Lorazepam, Lorbamate, Lorcainide Hydrochloride, Loreclezole, Loreinadol, lorglumide, Lormetazepam, Lornoxicam, lornoxicam, Lortalamine, Lorzafone, losartan, losigamone, losoxantrone, Losulazine Hydrochloride, loteprednol, lovastatin, loviride, Loxapine, Loxoribine, lubeluzole, Lucanthone Hydrochloride, Lufironil, Lurosetron Mesylate, lurtotecan, luteinizing hormone, lutetium, Lutrelin Acetate, luzindole, Lyapolate Sodium, Lycetamine, lydicamycin, Lydimycin, Lynestrenol, Lypressin, Lysine, lysofylline, lysostaphin, lytic peptides, Maduramicin, Mafenide, magainin 2 amide, Magnesium Salicylate, Magnesium Sulfate, magnolol, maitansine, Malethamer, mallotochromene, mallotoj aponin, Malotilate, malotilate, mangafodipir, manidipine, maniwamycin A, Mannitol, mannostatin A, manumycin E, manumycin F, mapinastine, Maprotiline, marimastat, Martek 8708, Martek 92211, Masoprocol, maspin, massetolide, matrilysin inhibitors, Maytansine, Mazapertine Succiniate, Mazindol, Mebendazole, Mebeverine Hydrochloride, Mebrofenin, Mebutamate, Mecamylamine Hydrochloride, Mechlorethamine Hydrochloride, Meclocycline, Meclofenamate Sodium, Mecloqualone, Meclori sone Dibutyrate, Medazepam Hydrochloride, Medorinone, Medrogestone, Medroxalol, Medroxyprogesterone, Medrysone, Meelizine Hydrochloride, Mefenamic Acid, Mefenidil, Mefenorex Hydrochloride, Mefexamide, Mefloquine Hydrochloride, Mefruside, Megalomicin Potassium Phosphate, Megestrol Acetate, Meglumine, Meglutol, Melengestrol Acetate, Melitracen Hydrochloride, Melphalan, Memotine Hydrochloride, Menabitan Hydrochloride, Menoctone, menogaril, Menotropins, Meobentine Sulfate, Mepartricin, Mepenzolate Bromide, Meperidine Hydrochloride, Mephentermine Sulfate, Mephenyloin, Mephobarbital, Mepivacaine Hydrochloride, Meprobamate, Meptazinol Hydrochloride, Mequidox, Meralein Sodium, merbarone, Mercaptopurine, Mercufenol Chloride, Mercury, Ammoniated, Merisoprol Hg 197, Meropenem, Mesalamine, Meseclazone, Mesoridazine, Mesterolone, Mestranol, Mesuprine Hydrochloride, Metalol Hydrochloride, Metaproterenol Polistirex, Metaraminol Bitartrate, Metaxalone, Meteneprost, meterelin, Metformin, Methacholine Chloride, Methacycline, Methadone Hydrochloride, Methadyl Acetate, Methalthiazide, Methamphetamine Hydrochloride, Methaqualone, Methazolamide, Methdilazine, Methenamine, Methenolone Acetate, Methetoin, Methicillin Sodium, Methimazole, methioninase, Methionine, Methisazone, Methixene Hydrochloride, Methocarbamol, Methohexital Sodium, Methopholine, Methotrexate, Methotrimeprazine, methoxatone, Methoxyflurane, Methsuximide, Methyclothiazide, Methyl Palmoxirate, Methylatropine Nitrate, Methylbenzethonium Chloride, Methyldopa, Methyldopate Hydrochloride, Methylene Blue, Methylergonovine Maleate, methylhistamine, R-alpha, methylinosine monophosphate, Methylphenidate Hydrochloride, Methylprednisolone, Methyltestosterone, Methynodiol Diacelate, Methysergide, Methysergide Maleate, Metiamide, Metiapine, Metioprim, metipamide, Metipranolol, Metizoline Hydrochloride, Metkephamid Acetate, metoclopramide, Metocurine Iodide, Metogest, Metolazone, Metopimazine, Metoprine, Metoprolol, Metoquizine, metrifonate, Metrizamide, Metrizoate Sodium, Metronidazole, Meturedepa, Metyrapone, Metyrosine, Mexiletine Hydrochloride, Mexrenoate Potassium, Mezlocillin, mfonelic Acid, Mianserin Hydrochloride, mibefradil, Mibefradil Dihydrochloride, Mibolerone, michellamine B, Miconazole, microcolin A, Midaflur, Midazolam Hydrochloride, midodrine, mifepristone, Mifobate, miglitol, milacemide, milameline, mildronate, Milenperone, Milipertine, milnacipran, Milrinone, miltefosine, Mimbane Hydrochloride, minaprine, Minaxolone, Minocromil, Minocycline, Minoxidil, Mioflazine Hydrochloride, miokamycin, mipragoside, mirfentanil, mirimostim, Mirincamycin Hydrochloride, Mirisetron Maleate, Mirtazapine, mismatched double stranded RNA, Misonidazole, Misoprostol, Mitindomide, Mitocarcin, Mitocromin, Mitogillin, mitoguazone, mitolactol, Mitomalcin, Mitomycin, mitonafide, Mitosper, Mitotane, mitoxantrone, mivacurium chloride, mivazerol, mixanpril, Mixidine, mizolastine, mizoribine, Moclobemide, modafinil, Modaline Sulfate, Modecainide, moexipril, mofarotene, Mofegiline Hydrochloride, mofezolac, molgramostim, Molinazone, Molindone Hydrochloride, Molsidomine, mometasone, Monatepil Maleate, Monensin, Monoctanoin, Montelukast Sodium, montirelin, mopidamol, moracizine, Morantel Tartrate, Moricizine, Morniflumate, Morphine Sulfate, Morrhuate Sodium, mosapramine, mosapride, motilide, Motretinide, Moxalactam Disodium, Moxazocine, moxiraprine, Moxnidazole, moxonidine, Mumps Skin Test Antigen, mustard anticancer agent, Muzolimine, mycaperoxide B, Mycophenolic Acid, myriaporone, Nabazenil, Nabilone, Nabitan Hydrochloride, Naboctate Hydrochloride, Nabumetone, N-acetyldinaline, Nadide, nadifloxacin, Nadolol, nadroparin calcium, nafadotride, nafamostat, nafarelin, Nafcillin Sodium, Nafenopin, Nafimidone Hydrochloride, Naflocort, Nafomine Malate, Nafoxidine Hydrochloride, Nafronyl Oxalate, Naftifine Hydrochloride, naftopidil, naglivan, nagrestip, Nalbuphine Hydrochloride, Nalidixate Sodium, Nalidixic Acid, nalmefene, Nalmexone Hydrochloride, naloxone+pentazocine, Naltrexone, Namoxyrate, Nandrolone Phenpropionate, Nantradol Hydrochloride, Napactadine Hydrochloride, napadisilate, Napamezole Hydrochloride, napaviin, Naphazoline Hydrochloride, naphterpin, Naproxen, Naproxol, napsagatran, Naranol Hydrochloride, Narasin, naratriptan, nartograstim, nasaruplase, Natamycin, nateplase, Naxagolide Hydrochloride, Nebivolol, Nebramycin, nedaplatin, Nedocromil, Nefazodone Hydrochloride, Neflumozide Hydrochloride, Nefopam Hydrochloride, Nelezaprine Maleate, Nemazoline Hydrochloride, nemorubicin, Neomycin Palmitate, Neostigmine Bromide, neridronic acid, Netilmicin Sulfate, neutral endopeptidase, Neutramycin, Nevirapine, Nexeridine Hydrochloride, Niacin, Nibroxane, Nicardipine Hydrochloride, Nicergoline, Niclosamide, Nicorandil, Nicotinyl Alcohol, Nifedipine, Nifirmerone, Nifluridide, Nifuradene, Nifuraldezone, Nifuratel, Nifuratrone, Nifurdazil, Nifurimide, Nifurpirinol, Nifurquinazol, Nifurthiazole, nilutamide, Nilvadipine, Nimazone, Nimodipine, niperotidine, niravoline, Niridazole, nisamycin, Nisbuterol Mesylate, nisin, Nisobamate, Nisoldipine, Nisoxetine, Nisterime Acetate, Nitarsone, nitazoxanide, nitecapone, Nitrafudam Hydrochloride, Nitralamine Hydrochloride, Nitramisole Hydrochloride, Nitrazepam, Nitrendipine, Nitrocycline, Nitrodan, Nitrofurantoin, Nitrofurazone, Nitroglycerin, Nitromersol, Nitromide, Nitromifene Citrate, Nitrous Oxide, nitroxide antioxidant, nitrullyn, Nivazol, Nivimedone Sodium, Nizatidine, Noberastine, Nocodazole, Nogalamycin, Nolinium Bromide, Nomifensine Maleate, Noracymethadol Hydrochloride, Norbolethone, Norepinephrine Bitartrate, Norethindrone, Norethynodrel, Norfloxacin, Norflurane, Norgestimate, Norgestomet, Norgestrel, Nortriptyline Hydrochloride, Noscapine, Novobiocin Sodium, N-substituted benzaimides, Nufenoxole, Nylestriol, Nystatin, O6-benzylguanine, Obidoxime Chloride, Ocaperidone, Ocfentanil Hydrochloride, Ocinaplon, Octanoic Acid, Octazamide, Octenidine Hydrochloride, Octodrine, Octreotide, Octriptyline Phosphate, Ofloxacin, Oformine, okicenone, Olanzapine, oligonucleotides, olopatadine, olprinone, olsalazine, Olsalazine Sodium, Olvanil, omeprazole, onapristone, ondansetron, Ontazolast, Oocyte maturation inhibitor, Opipramol Hydrochloride, oracin, Orconazole Nitrate, Orgotein, Orlislat, Ormaplatin, Ormetoprim, Ornidazole, Orpanoxin, Orphenadrine Citrate, osaterone, otenzepad, Oxacillin Sodium, Oxagrelate, oxaliplatin, Oxamarin Hydrochloride, oxamisole, Oxamniquine, oxandrolone, Oxantel Pamoate, Oxaprotiline Hydrochloride, Oxaprozin, Oxarbazole, Oxatomide, oxaunomycin, Oxazepam, oxcarbazepine, Oxendolone, Oxethazaine, Oxetorone Fumarate, Oxfendazole, Oxfenicine, Oxibendazole, oxiconazole, Oxidopamine, Oxidronic Acid, Oxifungin Hydrochloride, Oxilorphan, Oximonam, Oximonam Sodium, Oxiperomide, oxiracetam, Oxiramide, Oxisuran, Oxmetidine Hydrochloride, oxodipine, Oxogestone Phenpropionate, Oxolinic Acid, Oxprenolol Hydrochloride, Oxtriphylline, Oxybutynin Chloride, Oxychlorosene, Oxycodone, Oxymetazoline Hydrochloride, Oxymetholone, Oxymorphone Hydrochloride, Oxypertine, Oxyphenbutazone, Oxypurinol, Oxytetracycline, Oxytocin, ozagrel, Ozolinone, Paclitaxel, palauamine, Paldimycin, palinavir, palmitoylrhizoxin, Palmoxirate Sodium, pamaqueside, Pamatolol Sulfate, pamicogrel, Pamidronate Disodium, pamidronic acid, Panadiplon, panamesine, panaxytriol, Pancopride, Pancuronium Bromide, panipenem, pannorin, panomifene, pantethine, pantoprazole, Papaverine Hydrochloride, parabactin, Parachlorophenol, Paraldehyde, Paramethasone Acetate, Paranyline Hydrochloride, Parapenzolate Bromide, Pararosaniline Pamoate, Parbendazole, Parconazole Hydrochloride, Paregoric, Pareptide Sulfate, Pargyline Hydrochloride, parnaparin sodium, Paromomycin Sulfate, Paroxetine, parthenolide, Partricin, Paulomycin, pazelliptine, Pazinaclone, Pazoxide, pazufloxacin, pefloxacin, pegaspargase, Pegorgotein, Pelanserin Hydrochloride, peldesine, Peliomycin, Pelretin, Pelrinone Hydrochloride, Pemedolac, Pemerid Nitrate, pemirolast, Pemoline, Penamecillin, Penbutolol Sulfate, Penciclovir, Penfluridol, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentabamate, Pentaerythritol Tetranitrate, pentafuside, pentamidine, pentamorphone, Pentamustine, Pentapiperium Methylsulfate, Pentazocine, Pentetic Acid, Pentiapine Maleate, pentigetide, Pentisomicin, Pentizidone Sodium, Pentobarbital, Pentomone, Pentopril, pentosan, pentostatin, Pentoxifylline, Pentrinitrol, pentrozole, Peplomycin Sulfate, Pepstatin, perflubron, perfofamide, Perfosfamide, pergolide, Perhexiline Maleate, perillyl alcohol, Perindopril, perindoprilat, Perlapine, Permethrin, perospirone, Perphenazine, Phenacemide, phenaridine, phenazinomycin, Phenazopyridine Hydrochloride, Phenbutazone Sodium Glycerate, Phencarbamide, Phencyclidine Hydrochloride, Phendimetrazine Tartrate, Phenelzine Sulfate, Phenmetrazine Hydrochloride, Phenobarbital, Phenoxybenzamine Hydrochloride, Phenprocoumon, phenserine, phensuccinal, Phensuximide, Phentermine, Phentermine Hydrochloride, phentolamine mesilate, Phentoxifylline, Phenyl Aminosalicylate, phenylacetate, Phenylalanine, phenylalanyl ketoconazole, Phenylbutazone, Phenylephrine Hydrochloride, Phenylpropanolamine Hydrochloride, Phenylpropanolamine Polistirex, Phenyramidol Hydrochloride, Phenyloin, phosphatase inhibitors, Physostigmine, picenadol, picibanil, Picotrin Diolamine, picroliv, picumeterol, pidotimod, Pifamine, Pilocarpine, pilsicainide, pimagedine, Pimetine Hydrochloride, pimilprost, Pimobendan, Pimozide, Pinacidil, Pinadoline, Pindolol, pinnenol, pinocebrin, Pinoxepin Hydrochloride, pioglitazone, Pipamperone, Pipazethate, pipecuronium bromide, Piperacetazine, Piperacillin Sodium, Piperamide Maleate, piperazine, Pipobroman, Piposulfan, Pipotiazine Palmitate, Pipoxolan Hydrochloride, Piprozolin, Piquindone Hydrochloride, Piquizil Hydrochloride, Piracetam, Pirandamine Hydrochloride, pirarubicin, Pirazmonam Sodium, Pirazolac, Pirbenicillin Sodium, Pirbuterol Acetate, Pirenperone, Pirenzepine Hydrochloride, piretanide, Pirfenidone, Piridicillin Sodium, Piridronate Sodium, Piriprost, piritrexim, Pirlimycin Hydrochloride, pirlindole, pirmagrel, Pirmenol Hydrochloride, Pirnabine, Piroctone, Pirodavir, pirodomast, Pirogliride Tartrate, Pirolate, Pirolazamide, Piroxantrone Hydrochloride, Piroxicam, Piroximone, Pirprofen, Pirquinozol, Pirsidomine, Prenylamine, Pituitary, Posterior, Pivampicillin Hydrochloride, Pivopril, Pizotyline, placetin A, platinum compounds, platinum-triamine complex, Plicamycin, Plomestane, Pobilukast Edamine, Podofilox, Poisonoak Extract, Poldine Methyl sulfate, Poliglusam, Polignate Sodium, Polymyxin B Sulfate, Polythiazide, Ponalrestat, Porfimer Sodium, Porfiromycin, Potassium Chloride, Potassium Iodide, Potassium Permanganate, Povidone-Iodine, Practolol, Pralidoxime Chloride, Pramiracetam Hydrochloride, Pramoxine Hydrochloride, Pranolium Chloride, Pravadoline Maleate, Pravastatin (Pravachol), Prazepam, Prazosin, Prazosin Hydrochloride, Prednazate, Prednicarbate, Prednimustine, Prednisolone, Prednisone, Prednival, Pregnenolone Succiniate, Prenalterol Hydrochloride, Pridefine Hydrochloride, Prifelone, Prilocalne Hydrochloride, Prilosec, Primaquine Phosphate, Primidolol, Primidone, Prinivil, Prinomide Tromethamine, Prinoxodan, Prizidilol Hydrochloride, Proadifen Hydrochloride, Probenecid, Probicromil Calcium, Probucol, Procainamide Hydrochloride, Procaine Hydrochloride, Procarbazine Hydrochloride, Procaterol Hydrochloride, Prochlorperazine, Procinonide, Proclonol, Procyclidine Hydrochloride, Prodilidine Hydrochloride, Prodolic Acid, Profadol Hydrochloride, Progabide, Progesterone, Proglumide, Proinsulin Human, Proline, Prolintane Hydrochloride, Promazine Hydrochloride, Promethazine Hydrochloride, Propafenone Hydrochloride, propagermanium, Propanidid, Propantheline Bromide, Proparacaine Hydrochloride, Propatyl Nitrate, propentofylline, Propenzolate Hydrochloride, Propikacin, Propiomazine, Propionic Acid, propionylcarnitine, L-, propiram, propiram+paracetamol, propiverine, Propofol, Propoxycaine Hydrochloride, Propoxyphene Hydrochloride, Propranolol Hydrochloride, Propulsid, propyl bis-acridone, Propylhexedrine, Propyliodone, Propylthiouracil, Proquazone, Prorenoate Potassium, Proroxan Hydrochloride, Proscillaridin, Prostalene, prostratin, Protamine Sulfate, protegrin, Protirelin, protosufloxacin, Protriptyline Hydrochloride, Proxazole, Proxazole Citrate, Proxicromil, Proxorphan Tartrate, prulifloxacin, Pseudoephedrine Hydrochloride, Puromycin, purpurins, Pyrabrom, Pyrantel Pamoate, Pyrazinamide, Pyrazofurin, pyrazoloacridine, Pyridostigmine Bromide, Pyrilamine Maleate, Pyrimethamine, Pyrinoline, Pyrithione Sodium, Pyrithione Zinc, Pyrovalerone Hydrochloride, Pyroxamine Maleate, Pyr-rocaine, Pyrroliphene Hydrochloride, Pyrrolnitrin, Pyrvinium Pamoate, Quadazocine Mesylate, Quazepam, Quazinone, Quazodine, Quazolast, quetiapine, quiflapon, quinagolide, Quinaldine Blue, quinapril, Quinaprilat, Quinazosin Hydrochloride, Quinbolone, Quinctolate, Quindecamine Acetate, Quindonium Bromide, Quinelorane Hydrochloride, Quinestrol, Quinfamide, Quingestanol Acetate, Quingestrone, Quinidine Gluconate, Quinielorane Hydrochloride, Quinine Sulfate, Quinpirole Hydrochloride, Quinterenol Sulfate, Quinuclium Bromide, Quinupristin, Quipazine Maleate, Rabeprazole Sodium, Racephenicol, Racepinephrine, raf antagonists, Rafoxanide, Ralitoline, raloxifene, raltitrexed, ramatroban, Ramipril, Ramoplanin, ramosetron, ranelic acid, Ranimycin, Ranitidine, ranolazine, Rauwolfia Serpentina, recainam, Recainam Hydrochloride, Reclazepam, regavirumab, Regramostim, Relaxin, Relomycin, Remacemide Hydrochloride, Remifentanil Hydrochloride, Remiprostol, Remoxipride, Repirinast, Repromicin, Reproterol Hydrochloride, Reserpine, resinferatoxin, Resorcinol, retelliptine demethylated, reticulon, reviparin sodium, revizinone, rhenium Re 186 etidronate, rhizoxin, Ribaminol, Ribavirin, Riboprine, ribozymes, ricasetron, Ridogrel, Rifabutin, Rifametane, Rifamexil, Rifamide, Rifampin, Rifapentine, Rifaximin, RH retinamide, rilopirox, Riluzole, rimantadine, Rimcazole Hydrochloride, Rimexolone, Rimiterol Hydrobromide, rimoprogin, riodipine, Rioprostil, Ripazepam, ripisartan, Risedronate Sodium, risedronic acid, Risocaine, Risotilide Hydrochloride, rispenzepine, Risperdal, Risperidone, Ritanserin, ritipenem, Ritodrine, Ritolukast, ritonavir, rizatriptan benzoate, Rocastine Hydrochloride, Rocuronium Bromide, Rodocaine, Roflurane, Rogletimide, rohitukine, rokitamycin, Roletamicide, Rolgamidine, Rolicyprine, Rolipram, Rolitetracycline, Rolodine, Romazarit, romurtide, Ronidazole, ropinirole, Ropitoin Hydrochloride, ropivacaine, Ropizine, roquinimex, Rosaramicin, Rosoxacin, Rotoxamine, roxaitidine, Roxarsone, roxindole, roxithromycin, rubiginone B1, ruboxyl, rufloxacin, rupatidine, Rutamycin, ruzadolane, Sabeluzole, safingol, safironil, saintopin, salbutamol, R-, Salcolex, Salethamide Maleate, Salicyl Alcohol, Salicylamide, Salicylate Meglumine, Salicylic Acid, Salmeterol, Salnacediin, Salsalate, sameridine, sampatrilat, Sancycline, sanfetrinem, Sanguinarium Chloride, Saperconazole, saprisartan, sapropterin, saquinavir, Sarafloxacin Hydrochloride, Saralasin Acetate, SarCNU, sarcophytol A, sargramostim, Sarmoxicillin, Sarpicillin, sarpogrelate, saruplase, saterinone, satigrel, satumomab pendetide, Schick Test Control, Scopafungin, Scopolamine Hydrobromide, Scrazaipine Hydrochloride, Sdi 1 mimetics, Secalciferol, Secobarbital, Seelzone, Seglitide Acetate, selegiline, Selegiline Hydrochloride, Selenium Sulfide, Selenomethionine Se 75, Selfotel, sematilide, semduramicin, semotiadil, semustine, sense oligonucleotides, Sepazonium Chloride, Seperidol Hydrochloride, Seprilose, Seproxetine Hydrochloride, Seractide Acetate, Sergolexole Maleate, Serine, Sermetacin, Sermorelin Acetate, sertaconazole, sertindole, sertraline, setiptiline, Setoperone, sevirumab, sevoflurane, sezolamide, Sibopirdine, Sibutramine Hydrochloride, signal transduction inhibitors, Silandrone, silipide, silteplase, Silver Nitrate, simendan, Simtrazene, Simvastatin, Sincalide, Sinefungin, sinitrodil, sinnabidol, sipatrigine, sirolimus, Sisomicin, Sitogluside, sizofiran, sobuzoxane, Sodium Amylosulfate, Sodium Iodide I 123, Sodium Nitroprusside, Sodium Oxybate, sodium phenylacetate, Sodium Salicylate, solverol, Solypertine Tartrate, Somalapor, Somantadine Hydrochloride, somatomedin B, somatomedin C, somatrem, somatropin, Somenopor, Somidobove, sonermin, Sorbinil, Sorivudine, sotalol, Soterenol Hydrochloride, Sparfloxacin, Sparfosate Sodium, sparfosic acid, Sparsomycin, Sparteine Sulfate, Spectinomycin Hydrochloride, spicamycin D, Spiperone, Spiradoline Mesylate, Spiramycin, Spirapril Hydrochloride, Spiraprilat, Spirogermanium Hydrochloride, Spiromustine, Spironolactone, Spiroplatin, Spiroxasone, splenopentin, spongistatin 1, Sprodiamide, squalamine, Stallimycin Hydrochloride, Stannous Pyrophosphate, Stannous Sulfur Colloid, Stanozolol, Statolon, staurosporine, stavudine, Steffimycin, Stenbolone Acetate, stepronin, Stilbazium Iodide, Stilonium Iodide, stipiamide, Stiripentol, stobadine, Streptomycin Sulfate, Streptonicozid, Streptonigrin, Streptozocin, stromelysin inhibitors, Strontium Chloride Sr 89, succibun, Succimer, Succinylcholine Chloride, Sucralfate, Sucrosofate Potassium, Sudoxicam, Sufentanil, Sufotidine, Sulazepam, Sulbactam Pivoxil, Sulconazole Nitrate, Sulfabenz, Sulfabenzamide, Sulfacetamide, Sulfacytine, Sulfadiazine, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, sulfasalazine, Sulfasomizole, Sulfazamet, Sulfinalol Hydrochloride, sulfinosine, Sulfinpyrazone, Sulfisoxazole, Sulfomyxin, Sulfonterol Hydrochloride, sulfoxamine, Sulinldac, Sulmarin, Sulnidazole, Suloctidil, Sulofenur, sulopenem, Suloxifen Oxalate, Sulpiride, Sulprostone, sultamicillin, Sulthiame, sultopride, sulukast, Sumarotene, sumatriptan, Suncillin Sodium, Suproclone, Suprofen, suradista, suramin, Surfomer, Suricainide Maleate, Suritozole, Suronacrine Maleate, Suxemerid Sulfate, swainsonine, symakalim, Symclosene, Symetine Hydrochloride, synthetic glycosaminoglycans, Taciamine Hydrochloride, Tacrine Hydrochloride, Tacrolimus, Talampicillin Hydrochloride, Taleranol, Tali somycin, tallimustine, Talmetacin, Talniflumate, Talopram Hydrochloride, Talosalate, Tametraline Hydrochloride, Tamoxifen, Tampramine Fumarate, Tamsulosin Hydrochloride, Tandamine Hydrochloride, tandospirone, tapgen, taprostene, Tasosartan, tauromustine, Taxane, Taxoid, Tazadolene Succinate, tazanolast, tazarotene, Tazifylline Hydrochloride, Tazobactam, Tazofelone, Tazolol Hydrochloride, Tebufelone, Tebuquine, Technetium Tc 99 m Bicisate, Teclozan, Tecogalan Sodium, Teecleukin, Teflurane, Tegafur, Tegretol, Teicoplanin, telenzepine, tellurapyrylium, telmesteine, telmisartan, telomerase inhibitors, Teloxantrone Hydrochloride, Teludipine Hydrochloride, Temafloxacin Hydrochloride, Tematropium Methyl sulfate, Temazepam, Temelastine, temocapril, Temocillin, temoporfin, temozolomide, Tenidap, Teniposide, tenosal, tenoxicam, tepirindole, Tepoxalin, Teprotide, terazosin, Terbinafine, Terbutaline Sulfate, Terconazole, terfenadine, terflavoxate, terguride, Teriparatide Acetate, terlakiren, terlipressin, terodiline, Teroxalene Hydrochloride, Teroxirone, tertatolol, Tesicam, Tesimide, Testolactone, Testosterone, Tetracaine, tetrachlorodecaoxide, Tetracycline, Tetrahydrozoline Hydrochloride, Tetrami sole Hydrochloride, Tetrazolast Meglumine, tetrazomine, Tetrofosmin, Tetroquinone, Tetroxoprim, Tetrydamine, thaliblastine, Thalidomide, Theofibrate, Theophylline, Thiabendazole, Thiamiprine, Thiamphenicol, Thiamylal, Thiazesim Hydrochloride, Thiazinamium Chloride, Thiethylperazine, Thimerfonate Sodium, Thimerosal, thiocoraline, thiofedrine, Thioguanine, thiomarinol, Thiopental Sodium, thioperamide, Thioridazine, Thiotepa, Thiothixene, Thiphenamil Hydrochloride, Thiphencillin Potassium, Thiram, Thozalinone, Threonine, Thrombin, thrombopoietin, thrombopoietin mimetic, thymalfasin, thymopoietin receptor agonist, thymotrinan, Thyromedan Hydrochloride, Thyroxine 1 125, Thyroxine 1 131, Tiacrilast, Tiacrilast Sodium, tiagabine, Tiamenidine, tianeptine, tiapafant, Tiapamil Hydrochloride, Tiaramide Hydrochloride, Tiazofurin, Tibenelast Sodium, Tibolone, Tibric Acid, Ticabesone Propionate, Ticarbodine, Ticarcillin Cresyl Sodium, Ticlatone, ticlopidine, Ticrynafen, tienoxolol, Tifurac Sodium, Tigemonam Dicholine, Tigestol, Tiletamine Hydrochloride, Tilidine Hydrochloride, tilisolol, tilnoprofen arbamel, Tilorone Hydrochloride, Tiludronate Disodium, tiludronic acid, Timefurone, Timobesone Acetate, Timolol, tin ethyl etiopurpurin, Tinabinol, Tinidazole, Tinzaparin Sodium, Tioconazole, Tiodazosin, Tiodonium Chloride, Tioperidone Hydrochloride, Tiopinac, Tiospirone Hydrochloride, Tiotidine, tiotropium bromide, Tioxidazole, Tipentosin Hydrochloride, Tipredane, Tiprenolol Hydrochloride, Tiprinast Meglumine, Tipropidil Hydrochloride, Tiqueside, Tiquinamide Hydrochloride, tirandalydigin, Tirapazamine, tirilazad, tirofiban, tiropramide, titanocene dichloride, Tixanox, Tixocortol Pivalate, Tizanidine Hydrochloride, Tobramycin, Tocainide, Tocamphyl, Tofenacin Hydrochloride, Tolamolol, Tolazamide, Tolazoline Hydrochloride, Tolbutamide, Tolcapone, Tolciclate, Tolfamide, Tolgabide, lamotrigine, Tolimidone, Tolindate, Tolmetin, Tolnaftate, Tolpovidone 1 131, Tolpyrramide, Tolrestat, Tomelukast, Tomoxetine Hydrochloride, Tonazocine Mesylate, Topiramate, topotecan, Topotecan Hydrochloride, topsentin, Topterone, Toquizine, torasemide, toremifene, Torsemide, Tosifen, Tosufloxacin, totipotent stem cell factor, Tracazolate, trafermin, Tralonide, Tramadol Hydrochloride, Tramazoline Hydrochloride, trandolapril, Tranexamic Acid, Tranilast, Transcainide, translation inhibitors, traxanox, Trazodone Hydrochloride, Trazodone-HCL, Trebenzomine Hydrochloride, Trefentanil Hydrochloride, Treloxinate, Trepipam Maleate, Trestolone Acetate, tretinoin, Triacetin, triacetyluridine, Triafungin, Triamcinolone, Triampyzine Sulfate, Triamterene, Triazolam, Tribenoside, tricaprilin, Tricetamide, Trichlormethiazide, trichohyalin, triciribine, Tricitrates, Triclofenol piperazine, Triclofos Sodium, Triclonide, trientine, Trifenagrel, triflavin, Triflocin, Triflubazam, Triflumidate, Trifluoperazine Hydrochloride, Trifluperidol, Triflupromazine, Triflupromazine Hydrochloride, Trifluridine, Trihexyphenidyl Hydrochloride, Trilostane, Trimazosin Hydrochloride, trimegestone, Trimeprazine Tartrate, Trimethadione, Trimethaphan Camsylate, Trimethobenzamide Hydrochloride, Trimethoprim, Trimetozine, Trimetrexate, Trimipramine, Trimoprostil, Trimoxamine Hydrochloride, Triolein 1 125, Triolein 1 131, Trioxifene Mesylate, Tripamide, Tripelennamine Hydrochloride, Triprolidine Hydrochloride, Triptorelin, Trisulfapyrimidines, Troclosene Potassium, troglitazone, Trolamine, Troleandomycin, trombodipine, trometamol, Tropanserin Hydrochloride, Tropicamide, tropine ester, tropisetron, trospectomycin, trovafloxacin, trovirdine, Tryptophan, Tuberculin, Tubocurarine Chloride, Tubulozole Hydrochloride, tucarcsol, tulobuterol, turosteride, Tybamate, tylogenin, Tyropanoate Sodium, Tyrosine, Tyrothricin, tyrphostins, ubenimex, Uldazepam, Undecylenic Acid, Uracil Mustard, urapidil, Urea, Uredepa, uridine triphosphate, Urofollitropin, Urokinase, Ursodiol, valaciclovir, Valine, Valnoctamide, Valproate Sodium, Valproic Acid, valsartan, vamicamide, vanadeine, Vancomycin, vaninolol, Vapiprost Hydrochloride, Vapreotide, variolin B, Vasopressin, Vecuronium Bromide, velaresol, Velnacrine Maleate, venlafaxine, Veradoline Hydrochloride, veramine, Verapamil Hydrochloride, verdins, Verilopam Hydrochloride, Verlukast, Verofylline, veroxan, verteporfin, Vesnarinone, vexibinol, Vidarabine, vigabatrin, Viloxazine Hydrochloride, Vinblastine Sulfate, vinburnine citrate, Vincofos, vinconate, Vincristine Sulfate, Vindesine, Vindesine Sulfate, Vinepidine Sulfate, Vinglycinate Sulfate, Vinleurosine Sulfate, vinorelbine, vinpocetine, vintoperol, vinxaltine, Vinzolidine Sulfate, Viprostol, Virginiamycin, Viridofulvin, Viroxime, vitaxin, Volazocine, voriconazole, vorozole, voxergolide, Warfarin Sodium, Xamoterol, Xanomeline, Xanoxate Sodium, Xanthinol Niacinate, xemilofiban, Xenalipin, Xenbucin, Xilobam, ximoprofen, Xipamide, Xorphanol Mesylate, Xylamidine Tosylate, Xylazine Hydrochloride, Xylometazoline Hydrochloride, Xylose, yangambin, zabicipril, zacopride, zafirlukast, Zalcitabine, zaleplon, zalospirone, Zaltidine Hydrochloride, zaltoprofen, zanamivir, zankiren, zanoterone, Zantac, Zarirlukast, zatebradine, zatosetron, Zatosetron Maleate, zenarestat, Zenazocine Mesylate, Zeniplatin, Zeranol, Zidometacin, Zidovudine, zifrosilone, Zilantel, zilascorb, zileuton, Zimeldine Hydrochloride, Zinc Undecylenate, Zindotrine, Zinoconazole Hydrochloride, Zinostatin, Zinterol Hydrochloride, Zinviroxime, ziprasidone, Zobolt, Zofenopril Calcium, Zofenoprilat, Zolamine Hydrochloride, Zolazepam Hydrochloride, zoledronie acid, Zolertine Hydrochloride, zolmitriptan, zolpidem, Zomepirac Sodium, Zometapine, Zoniclezole Hydrochloride, Zonisamide, zopiclone, Zopolrestat, Zorbamyciin, Zorubicin Hydrochloride, zotepine, Zucapsaicin or its pharmaceutically acceptable salts thereof.

As indicated the pharmaceutical formulations as disclosed herein may comprise auxiliary excipients such as for example diluents, binders, lubricants, surfactants, disintegrants, plasticisers, anti-tack agents, opacifying agents, pigments, and such like. As will be appreciated by those skilled in the art, the exact choice of excipient and their relative amounts will depend to some extent on the final dosage form.

In one aspect examples of active ingredients comprise drugs such as but not limited to lidocaine, capsaicin, Tricyclic Antidepressants (not limited to such as amitriptyline, imipramine, nortriptyline, desipramine, doxepin, etc.), SNRIs and SSRIs (not limited to such as duloxetine, venlafaxine, fluoxetine, milnacipran etc.), NSAIDS (not limited to diclofenac, aspirin, naproxen, ibuprofen, ketoprofen, celecoxib, meloxicam, etc.), acetaminophen, cox-2 inhibitors (not limited to celecoxib, etc.), anticonvulsants (not limited to such as carbamazepine, gabapentin, lamotrigine, pregabalin, oxcarbazepine, lamotrigine, etc.), valproic acid, menthol, camphor, methyl salicylate, salicylates, corticosteroid drugs (not limited to such as triamcinolone, methylprednisolone, cortisone, prednisone, dexamethasone, etc.), opioid, etc.

In one aspect transdermal delivery system and/or topical delivery system comprising drug combination of two or more drugs such as but not limited to THC, CBD, lidocaine, menthol, capsaicin, methyl salicylate, etc. Examples of drug combination of two or more drugs for transdermal delivery systems and/or topical delivery system includes such as but not limited to THC, CBD, lidocaine and combinations thereof, THC, CBD, menthol and combinations thereof, THC, CBD, capsaicin and combinations thereof, THC, CBD, methyl salicylate and combinations thereof, etc.

In another aspect transdermal delivery system and/or topical delivery system comprising drug combination of two or more drugs such as but not limited to THC, CBD, antidepressant drug, NSAIDS, anticonvulsants drug, corticosteroid drug, pain relievers, etc. Examples of drug combination of two or more drugs for transdermal delivery systems and/or topical delivery system includes such as but not limited to THC, CBD, antidepressant drug and combinations thereof, THC, CBD and anticonvulsant drug and combinations thereof, THC, CBD, corticosteroid drug and combinations thereof, THC, CBD, NSAID drug and combinations thereof, etc.

As indicated the pharmaceutical formulations as disclosed herein may comprise auxiliary excipients such as for example diluents, binders, lubricants, surfactants, disintegrants, plasticizers, tackifiers, opacifying agents, pigments, and such like. As will be appreciated by those skilled in the art, the exact choice of excipient and their relative amounts will depend to some extent on the final transdermal or topical dosage form.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Pharmaceutical Compositions

According to certain embodiments described herein, pharmaceutical composition or transdermal formulation and/or topical formulation of contains active agents such as cannabinoids, and derivatives of these compounds. More preferably transdermal and/or topical formulation may include active agents such as CBD and/or THC, and derivatives of these compounds.

One embodiment of the present disclosure can be a transdermal drug delivery system which may include without any limitation to transdermal formulation, transdermal patches, microneedles, iontophoresis, metered dose transdermal spray, metered dose transdermal gel, transdermal aerosols, transdermal film forming formulations.

According to certain embodiments described herein, pharmaceutical composition or transdermal formulation of contains active agents such as CBD, THC, psilocybin, psilocin, lysergic acid diethylamine (LSD), and/or ibogaine, and derivatives of these compounds. More preferably transdermal formulation may include active agents such as CBD, THC, psilocybin, psilocin, lysergic acid diethylamine (LSD), and/or ibogaine, and derivatives of these compounds.

One embodiment of the present disclosure can be a transdermal drug delivery system which may include without any limitation to transdermal formulation, transdermal patches, topical formulation, microneedles, iontophoresis, metered dose transdermal spray, film forming formulation, transdermal aerosols.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch, metered dose transdermal system, sachet, etc. Transdermal formulations which includes matrix patches without any limitations like adhesive matrix patch, drug in adhesive matrix patch, non-adhesive matrix patch, a transdermal matrix formulation as drug in adhesive matrix patch is preferred. Other transdermal formulations include transdermal gel, transdermal meter dose spray, transdermal meter dose aerosols, transdermal film forming formulation, microneedles.

Without any limitation, transdermal patch may include all transdermal drug delivery systems stated in art preferably but not limited to a multilayer transdermal matrix system, reservoir patch, matrix patch, bilayer matrix patch, multilayer matrix patch, microreservoir patch, adhesive systems, transdermally applicable tape and other.

In certain embodiments of the present disclosure, a pharmaceutical composition, such as a transdermal patch, comprises transdermal formulation containing active agents such as CBD and/or THC, and derivatives of these compounds contained in a reservoir or a matrix, and an adhesive which allows the pharmaceutical composition, such as a transdermal patch, to adhere to the skin, allowing the passage of the active agents such as CBD and/or THC, and derivatives of these compounds from the pharmaceutical composition, such as a transdermal patch, through the skin of the patient. The transdermal delivery system can be occlusive, semi-occlusive or non-occlusive, and can be adhesive or non-adhesive.

The transdermal formulation comprising active agents such as CBD and/or THC, and derivatives of these compounds can be incorporated within the patch and patch can be applied topically to the skin surface. The patch can be left on the subject for any suitable period of time.

In some embodiments, the pharmaceutical composition, such as a transdermal patch, provide for a constant rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical composition, such as a transdermal patch, described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical composition, such as a transdermal patch, described herein provide a constant blood serum level of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical composition, such as a transdermal patch, described herein provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical composition, such as a transdermal patch, described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical composition described herein provide a plasma concentration of the active components of the transdermal patches in a therapeutic range in a patient over a predetermined time. In exemplary embodiments as disclosed herein, the pharmaceutical composition, such as a transdermal patch, provides a blood serum level of active agent selected from without any limitation, of, for example, about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, about 5 μg/mL, and ranges thereof. In one aspect, pharmaceutical composition, such as a transdermal patch, provides a blood serum level of active agent in the range of 0.01 ng/mL-400 ng/mL. In another aspect, pharmaceutical composition, such as a transdermal patch, provides a blood serum level of active agent in the range of 0.01 ng/mL-100 ng/mL. In yet another aspect pharmaceutical composition, such as a transdermal patch, provides a blood serum level of active agent in the range of from 0.01-1 ng/ml to 1-100 ng/ml to 100-500 ng/ml to 500-1000 ng/ml to 1000-5000 ng/ml.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation comprising such as CBD and/or THC, and derivatives of these compounds can be topically applied to the skin surface for topical delivery of such CBD and/or THC, and derivatives of these compounds.

One embodiment of the present disclosure can be a topical drug delivery system which may include without any limitation to topical patches, topical formulation, metered dose topical spray, topical film forming formulation, topical drug-in-adhesive patches, topical matrix patches, topical aerosols, metered dose topical gel.

Multilayer Polymer Drug Matrix System: For instance, a transdermal drug delivery system is contemplated where the active substance matrix is constructed using water soluble polymers, which is then coated on the adhesive layer. Further, the active substance reservoir can be prepared as a polymer matrix. In addition, the active substance reservoir can be confined on the skin facing side of the transdermal drug delivery system by an active substance permeable membrane and on the opposite side from the skin by an active substance impermeable layer followed by adhesive layer.

The disclosure provides a transdermal drug delivery system comprising an active substance matrix containing area is a double or multilayered active substance matrix. In another embodiment, the active substance, CBD/PSI is in the simplest case dispersed, coarsely, colloidally or molecularly, in a solution or melt of base polymers. In the further a transdermal drug delivery system manufacturing techniques, the CBD/PSI is in the form of supersaturated solution, nano-emulsion or nano-suspension, amorphous, crystalline, co-crystals, coated with base polymers or solubilize in polymers using hot melt extrusion process.

The disclosure also includes such embodiments where the CBD/PSI matrix has a two or multi-layered structure, also called multi-laminate drug in adhesive patch. For example, the various matrix layers may contain polymer constitutes from the above-mentioned polymers. In this case, the matrix layers are differing from each other's in the term of polymer or pressure sensitive or hot melt polymers composition, CBD/PSI concentration, different permeating enhancers or solubilizers. The layers can be separated using semi-permeable membrane between two distinct drug-in-adhesive layers or multiple drug-in-adhesive layers under a single backing film. The term polymer film includes polymer without any limitation pressure sensitive adhesive and/or non-adhesive polymer.

In one aspect the disclosure further provides a polymer matrix formulation comprising CBD/PSI and a polymeric vehicle system. The vehicle system can include solvents (e.g., a solubilizer), permeability enhancing excipients and polymer or gelling agent or thickening agent, if required acid or base for pH adjustment.

Pretreatment: Various approaches have been used to open the barrier property of stratum corneum for drug permeation enhancement. Pretreatment with the use of chemical penetration enhancers is one of the techniques employed. The pretreatment has a potential to modulate the outermost layer of the skin reversibly and facilitate the drug uptake. Penetration enhancers act on lipid and protein regions in combination or alone on each region.

Penetration enhancers may be incorporated into the formulations described herein (e.g., transdermal drug delivery systems including drug in adhesive layers and separate adhesive and drug containing layers), however, it can lead to some incompatibility or interactions within the ingredients. Therefore, the present disclosure provides the alternative method of skin penetration enhancement as to preparation/pretreatment the skin with some penetration enhancers or a combination of penetration enhancers before the patch application.

Pretreatment applications described herein include application of a gel/spray/solution/wetting agent/soaked swab/soaked cotton ball/soaked gauzes to the skin prior to application of drug containing product, intended to be a patch. However, it is to be understood that the pretreatment composition can include another topical dosage form, solution gel, cream, etc. For instance, the pretreatment composition can be its own individual patch, such as CuradMediplast, a 40% salicylic acid patch, or a placebo patch comprising non-volatile components such as acrylic, silicone, or PIB adhesives or combinations thereof with an optional addition of a skin permeation enhancers to promote delivery of an active pharmaceutical ingredient through the skin.

The present disclosure provides a pretreatment composition wherein the penetration enhancers are incorporated in the form the topical dosage form as solution, gel, cream, spray, composition preferably but not limited to gel can be incorporated in a reservoir patch.

Topical Formulations: Topical formulation which includes liquids for example without any limitation like solutions, suspensions, dispersions, emulsion. Topical formulation includes semisolids for example without any limitations like gels, ointments, emulsions, creams, suspension, paste, lotion, balm. Liquid formulation and/or gel formulation incorporated in without any limitation to topical patch, metered dose topical system, sachet, etc. Topical formulations which include polymer matrix patch without any limitations like adhesive matrix patch, non-adhesive matrix, drug-in-adhesive matrix patch, a topical matrix formulation as drug in adhesive matrix patch is preferred. Other topical formulations include such as but not limited to topical gel, metered dose topical spray, metered dose topical aerosols, topical film forming formulation.

Without any limitation, topical patch may include all topical drug delivery systems stated in art preferably but not limited to reservoir patch, matrix patch, bilayer matrix patch, multilayer matrix patch, microreservoir patch, adhesive systems, topically applicable tape and other.

In certain embodiments of the present disclosure, a topical patch comprises topical formulation containing active agents such as diclofenac and/or CBD and/or THC, and derivatives of these compounds contained in a reservoir or a matrix, and an adhesive which allows the topical patch to adhere to the skin, allowing the passage of the active agents such as diclofenac and/or CBD and/or THC, and derivatives of these compounds from the topical patch to the skin of the patient. The topical delivery system can be occlusive, semi-occlusive or non-occlusive, and can be adhesive or non-adhesive.

The topical formulation comprising active agents such as diclofenac and/or CBD and/or THC, and derivatives of these compounds can be incorporated within the patch and patch can be applied topically to the skin surface. The patch can be left on the subject for any suitable period of time.

The transdermal formulation and/or topical formulation of some embodiments of the present disclosure may include carriers or ingredients in effective amount either alone or in combinations thereof without any limitation to the following carriers or ingredients such as solvents, gelling agents, polymers, pressure sensitive adhesive polymers, adhesive polymers biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, bulking agents, excipients, material to prepare patch, material to prepare matrix patch, material to prepare reservoir patch etc.

Active agents may be dissolved, suspended, dispersed or uniformly mixed in the above stated single carrier, mixture of carriers and combinations of carrier. Any combination of two or more drugs such as such as CBD and/or THC, and derivatives of these compounds may be dissolved, suspended, dispersed or uniformly mixed in the above stated single carrier, mixture of carriers and combinations of carrier.

The desired optimum transdermal and/or topical formulation of such as CBD and/or THC, and derivatives of these compounds alone or in combinations thereof may comprise without any limitation to following carriers as stated from example 1 to example 12 either alone or in combinations thereof.

According to certain embodiments, transdermal and/or topical compositions described herein are for the treatment and/or prevention and/or control of, for example, chronic pain.

### Indications

One embodiment of the disclosure is the pharmaceutical composition for use in the treatment of chronic pain according to the disclosure, wherein the composition contains active agent as disclosed herein, and wherein the composition is administered every other day, daily, twice daily, three times daily or four times daily for a period of at least one day, at least one week, anytime between one week to one year, at least one year, or longer. One embodiment of the disclosure is the pharmaceutical composition for use in the treatment of chronic pain according to the disclosure, wherein the composition is administered every other day, daily, twice daily, three times daily or four times daily for a period of at least one day, at least one week, anytime between one week to one year, at least one year, or longer. This way, a continuous decrease of (peripheral) neuropathic pain, inflammatory pain, musculoskeletal pain, pain due to muscle spasms, and/or other chronic pain states is achieved upon administering the pharmaceutical composition of the disclosure to a patient suffering from chronic pain.

In one embodiment, the pharmaceutical composition for use in the treatment of chronic pain according to the disclosure is a pharmaceutical transdermal composition wherein the use is the transdermal use in the treatment of chronic pain according to the disclosure.

In one embodiment, the pharmaceutical composition for use in the treatment of chronic pain according to the disclosure is a pharmaceutical topical composition wherein the use is the topical use in the treatment of chronic pain according to the disclosure. Here, topical composition will be topically applied to intact skin area experiencing pain in the treatment of chronic pain.

In one embodiment, the pharmaceutical composition for use in the treatment of chronic pain according to the disclosure is a pharmaceutical transdermal composition wherein the use is the transdermal use on intact skin of the treated person in the treatment of chronic pain according to the disclosure.

In one embodiment, the pharmaceutical composition for use in the treatment of chronic pain according to the disclosure, is a pharmaceutical transdermal composition wherein the use is the transdermal use on healthy intact skin of the treated person in the treatment of chronic pain according to the disclosure. Here, intact skin and healthy intact skin have their common scientific meaning and here refer to non-injured skin free of e.g., ulcers, wounds, lesions, cuts, and refer to skin comprising a closed outer layer of epidermis.

One embodiment of the disclosure is a pharmaceutical composition according to the disclosure or provided by the method of the disclosure, for use in the treatment of chronic pain according to the disclosure, wherein the chronic pain is neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, or combinations thereof.

In one embodiment, the pharmaceutical composition for use in the treatment of chronic pain according to the disclosure is the pharmaceutical composition, wherein the chronic pain is peripheral neuropathic pain.

One embodiment of the disclosure is a pharmaceutical composition according to the disclosure or provided by the method of the disclosure, for use in the treatment of chronic pain according to the disclosure, wherein the chronic pain is neuropathic pain selected from peripheral neuropathy caused by diabetes type 1 or 2, or induced by a noxious substance such as alcohol, due to vitamin B1, B6 and/or B12 deficiency, hypervitaminosis B6, hypothyroidism, chemotherapeutic compound such as paclitaxel or a taxane derivative, vincristine or a vinca alkaloid, cisplatin or a platinum derivate, drug-induced neuropathy, a compound for the treatment of infectious disease such as streptomycin, didanosine or zalcitabine, a chemically toxic compound, trigeminal neuralgia, post-herpetic neuralgia, intercostal neuralgia, entrapment neuropathy such as carpal tunnel syndrome, tarsal tunnel syndrome, abdominal cutaneous nerve entrapment syndrome, sciatic pain chronic idiopathic sensory neuropathy, small fiber neuropathy, hereditary motor and sensory neuropathies, chronic inflammatory demyelinating polyneuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and a mono- and polyneuropathy, complex regional pain syndrome type I and II (reflex sympathetic dystrophy), central neuropathic pain such as thalamic neuropathy, spinal cord injury neuropathy, post stroke pain, multiple sclerosis, multiple sclerosis neuropathy, syringomyelia, a spinal cord tumor, phantom limb pain, restless genital syndrome with pain, post-surgical scar pain including scar pain after cardiac surgery and mastectomy.

One embodiment of the disclosure is a pharmaceutical composition according to the disclosure or provided by the method of the disclosure, for use in the treatment of chronic pain according to the disclosure, wherein the dosing frequency of the pharmaceutical composition is between once every other day and eight times daily, preferably six, five, four, three, two or one times daily.

One embodiment of the disclosure is the pharmaceutical composition for use in the treatment of chronic pain according to the disclosure, wherein the pharmaceutical composition is administered during a period of at least one day, preferably at least one week, more preferably at least one month, most preferably at least one year, preferably the pharmaceutical composition is administered for one to ten years, more preferably the pharmaceutical composition is administered chronically. It is to be understood that it is part of the disclosure that the pharmaceutical composition for use in the treatment of chronic pain according to the disclosure is administered to patients suffering from chronic pain for the rest of their lifespan. This way, the chronic pain is at least less intense and preferably patients are relieved from the chronic pain to a large extent or even completely.

### CNS Disorders

According to the present disclosure, the methods, therapeutic agents and compositions of this invention are useful in treating a CNS-related condition or disorder in a subject, including but not limited to, a psychiatric disorder, a neurological disorder, a seizure disorder, a neuroinflammatory disorder, a sensory deficit disorder, pain, a neurodegenerative disease and/or disorder, a neuroendocrine disorder and/or dysfunction, a female sex dysfunction and/or a neurodegenerative disease and/or disorder.

### Psychiatric Disorders

### Mood Disorders

Provided herein are methods, therapeutic agents and compositions for treating a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter. Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). The risk of suicide among those with the disorder is high at greater than 6% over 20 years, while self-harm occurs in 30-40%.

Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

Premenstrual dysphoric disorder (PMDD) refers to a severe, at times disabling extension of premenstrual syndrome (PMS). PMDD causes extreme mood shifts with symptoms that typically begin seven to ten days before a female's period starts and continues for the first few days of a female's period. Symptoms include sadness or hopelessness, anxiety or tension, extreme moodiness, and marked irritability or anger.

### Anxiety Disorders

Also provided herein are methods, therapeutic agents and compositions for treating an anxiety disorder. Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And, in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Eating Disorders

The compounds described herein can be used in a method described herein, for example in the treatment of a disorder described herein such as an eating disorder. Eating disorders feature disturbances in eating behavior and weight regulation, and are associated with a wide range of adverse psychological, physical, and social consequences. An individual with an eating disorder may start out just eating smaller or larger amounts of food, but at some point, their urge to eat less or more spirals out of control. Eating disorders may be characterized by severe distress or concern about body weight or shape, or extreme efforts to manage weight or food intake. Eating disorders include anorexia nervosa, bulimia nervosa, binge-eating disorder, cachexia, and their variants. Individuals with anorexia nervosa typically see themselves as overweight, even when they are underweight. Individuals with anorexia nervosa can become obsessed with eating, food, and weight control. Individuals with anorexia nervosa typically weigh themselves repeatedly, portion food carefully, and eat very small quantities of only certain foods. Individuals with anorexia nervosa may engage in binge eating, followed by extreme dieting, excessive exercise, self-induced vomiting, or misuse of laxatives, diuretics, or enemas. Symptoms include extremely low body weight, severe food restriction, relentless pursuit of thinness and unwillingness to maintain a normal or healthy weight, intense fear of gaining weight, distorted body image and self-esteem that is heavily influenced by perceptions of body weight and shape, or a denial of the seriousness of low body weight, lack of menstruation among girls and women. Other symptoms include the thinning of the bones, brittle hair and nails, dry and yellowish skin, growth of fine hair all over the body, mild anemia, muscle wasting, and weakness, severe constipation, low blood pressure or slowed breathing and pulse, damage to the structure and function of the heart, brain damage, multi-organ failure, drop in internal body temperature, lethargy, sluggishness, and infertility. Individuals with bulimia nervosa have recurrent and frequent episodes of eating unusually large amounts of food and feel a lack of control over these episodes. This binge eating is followed by behavior that compensates for the overeating such as forced vomiting, excessive use of laxatives or diuretics, fasting, excessive exercise, or a combination of these behaviors.

Unlike anorexia nervosa, people with bulimia nervosa usually maintain what is considered a healthy or normal weight, while some are slightly overweight. But like people with anorexia nervosa, they typically fear gaining weight, want desperately to lose weight, and are unhappy with their body size and shape. Usually, bulimic behavior is done secretly because it is often accompanied by feelings of disgust or shame. The binge eating and purging cycle can happen anywhere from several times a week to many times a day. Other symptoms include chronically inflamed and sore throat, swollen salivary glands in the neck and jaw area, worn tooth enamel, and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (that can lead to a heart attack or stroke).

Individuals with binge-eating disorder lose control over their eating. Unlike bulimia nervosa, periods of binge eating are not followed by compensatory behaviors like purging, excessive exercise, or fasting. Individuals with binge-eating disorder often are overweight or obese. Obese individuals with binge-eating disorder are at higher risk for developing cardiovascular disease and high blood pressure. They also experience guilt, shame, and distress about their binge eating, which can lead to more binge eating.

Cachexia is also known as “wasting disorder,” and is an eating-related issue experienced by many cancer patients. Individuals with cachexia may continue to eat normally, but their body may refuse to utilize the vitamins and nutrients that it is ingesting, or they will lose their appetite and stop eating. When an individual experiences loss of appetite and stops eating, they can be considered to have developed anorexia nervosa.

### Epilepsy

The therapeutic agents and compositions described herein can be used for example in the treatment of a disorder described herein such as epilepsy, status epilepticus, or seizure, for example as described in WO2013/112605 and WO/2014/031792, the contents of which are incorporated herein in their entirety.

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

The therapeutic agents and compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Psychotic Disorders

Also provided herein are methods, therapeutic agents and compositions for treating a psychotic disorder. The term “psychotic disorders” as used herein refers to a group of illnesses that affect the mind. These illnesses alter a patient's ability to think clearly, make good judgments, respond emotionally, communicate effectively, understand reality, and behave appropriately. When symptoms are severe, patient with psychotic disorders have difficulty staying in touch with reality and are often unable to meet the ordinary demands of daily life. Psychotic disorders include but are not limited to, schizophrenia, schizophreniform disorder, schizo-affective disorder, delusional disorder, brief psychotic disorder, shared psychotic disorder, psychotic disorder due to a general medical condition, substance-induced psychotic disorder or psychotic disorders not otherwise specified (Diagnostic and Statistical Manual of Mental Disorders, Ed. 4th, American Psychiatric Association, Washington, D.C. 1994).

### Impulse Control Disorders

Also provided herein are methods, therapeutic agents and compositions for treating an impulse control disorder. The term “impulse control disorders” as used herein refers to a class of psychiatric disorders characterized by impulsivity, i.e., failure to resist a temptation, an urge of an impulse. Five behavioral stages characterize impulsivity: an impulse, growing tension, pleasure on acting, relief from the urge and finally guilt (which may or may not arise).

Many psychiatric disorders feature impulsivity, including substance-related disorders, behavioral addictions, attention deficit hyperactivity disorder, antisocial personality disorder, borderline personality disorder, conduct disorder and some mood disorders. The fifth edition of the American Psychiatric Association's Diagnostic and statistical manual of mental disorders (DSM-5) that was published in 2013 includes a new chapter (not in DSM-IV-TR) on Disruptive, Impulse-Control, and Conduct Disorders covering disorders “characterized by problems in emotional and behavioral self-control.” It also includes Impulse-Control Disorders Not Elsewhere Classified, which encompasses intermittent explosive disorder, pyromania, and kleptomania.

### Adjustment Disorder

Adjustment Disorders are the development of emotional or behavioral symptoms in response to an identifiable stressor(s) occurring within 3 months of the onset of the stressor(s). These symptoms include: experiencing more stress than normally expected in response to a stressful life event and/or having stress that causes significant problems in relationships, at work, or at school; and the symptoms are NOT the result of another mental health disorder or part of normal grieving.

Adjustment Disorder is typically linked to stressful events such as but not limited to: the death of a loved one; medical diagnosis (example: cancer), health problems either in oneself or a loved one; financial concerns; moving; divorce; sexuality discoveries; family problems, unexpected life events.

As provided herein the following types of Adjustment Disorder include, for example, the following: With Depressed Mood—symptoms include feeling sad, tearful and hopeless, and experiencing a lack of pleasure in things you used to enjoy; With Anxiety—symptoms mainly include nervousness, stress, worry, difficulty concentrating or remembering things, and feeling overwhelmed (Children with AD with anxiety may strongly fear being separated from their parents and loved ones); With mixed Anxiety and Depressed Mood—a combination of above; With Disturbance of Conduct—symptoms usually involve behavioral problems, such as fighting or reckless driving. Youths may skip school or vandalize property; With mixed disturbance of emotions and conduct—all of the above; Unspecified—symptoms don't fit with the above types of adjustment disorders, but often include physical problems, problems with family, friends, work, school.

Adjustment disorders can be:
Acute. Signs and symptoms last six months or less. They should ease once the stressor is removed.
Persistent (chronic). Signs and symptoms last more than six months. They continue to bother the patient and disrupt the patient's life.

### Neuropsychiatric Disorders

Also provided herein are methods, therapeutic agents and compositions for treating and/or preventing neuropsychiatric disorders. In certain embodiments, the neuropsychiatric condition is selected from the group consisting of addiction (e.g., a weight management disorder), anxiety, apathy, attention (e.g., the lack thereof), and depression (e.g., moderate depression, prolonged grief disorder (PGD), social anxiety, post-surgical depression, or depression from chronic pain).

In some embodiments, the mental, the behavioral, or the neuropsychiatric condition is PTSD, constructive impulsivity, a phobia, a fear, or the like.

In some embodiments, the mental, the behavioral, or the neuropsychiatric condition is depression or a depression disorder.

In some embodiments, the mental, the behavioral, or the neuropsychiatric condition is major depressive disorder.

In some embodiments, the mental, the behavioral, or the neuropsychiatric condition is prolonged grief disorder (PGD), social anxiety, post-surgical depression, depression from chronic pain.

In some embodiments, the mental, the behavioral, or the neuropsychiatric condition is prolonged grief disorder (PGD), such as, for example, following the death of a loved one.

In some embodiments, the mental, the behavioral, or the neuropsychiatric condition is social anxiety.

In some embodiments, the mental, the behavioral, or the neuropsychiatric condition is post-surgical depression. In some embodiments, the mental, the behavioral, or the neuropsychiatric condition is depression from chronic pain. In some embodiments, the mental, the behavioral, or the neuropsychiatric condition is a weight management disorder. In some embodiments, the individual maintains a desired body weight, controls weight gain, has increased weight loss, has diminished food addition, has reduced food cravings, has curbed impulsive eating, has increased metabolism, and/or decreases their caloric intake (e.g., through lower calorie foods).

### Neurological Disorders and Neurodevelopmental Disorders

Also provided herein are methods, therapeutic agents and compositions for treating and/or preventing neurodevelopment disorders. Neurodevelopmental disorders are a group of disorders in which the development of the central nervous system is disturbed. This can include developmental brain dysfunction, which can manifest as neuropsychiatric problems or impaired motor function, learning, language or non-verbal communication.

Non-limiting examples of neurodevelopmental disorder include autism, autistic disorder, autistic spectrum disorder, Asperger syndrome, Pediatric Autoimmune Neuropsychiatric Disorders Associated with Streptococcal infections (PANDAS); Rett syndrome; Williams syndrome; Renpenning's syndrome; fragile X syndrome; Down syndrome; Prader-Willi syndrome; Sotos syndrome; Tuberous sclerosis complex (TSC); Timothy syndrome; Joubert syndrome; holoprosencephaly; Hirschsprung's disease; intestinal neuronal dysplasia; and Williams syndrome, pervasive developmental disorder, attention deficit hyperactivity disorder, deficits in attention, motor control and perception (DAMP), schizophrenia, obsessive-compulsive disorder, disorders affecting emotion, learning ability, and memory.

### Neurodegenerative Disorders

Also provided herein are methods, therapeutic agents and compositions for treating and/or preventing a neurodegenerative disease. The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion (including for the treatment and prevention and prevention of seizures that are caused by schizoaffective disorder or by drugs used to treat schizophrenia); benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; drug-induced or medication-induced Parkinsonism (such as neuroleptic-induced acute akathisia, acute dystonia, Parkinsonism, or tardive dyskinesia, neuroleptic malignant syndrome, or medication-induced postural tremor); epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating a neurodegenerative disease also include treating loss of neuronal function characteristic of neurodegenerative disorder.

### Neuroendocrine Disorders

Also provided herein are methods, therapeutic agents and compositions that can be used for treating neuroendocrine disorders and dysfunction. As used herein, “neuroendocrine disorder” or “neuroendocrine dysfunction” refers to a variety of conditions caused by imbalances in the body's hormone production directly related to the brain. Neuroendocrine disorders involve interactions between the nervous system and the endocrine system. Because the hypothalamus and the pituitary gland are two areas of the brain that regulate the production of hormones, damage to the hypothalamus or pituitary gland, e.g., by traumatic brain injury, may impact the production of hormones and other neuroendocrine functions of the brain. In some embodiments, the neuroendocrine disorder or dysfunction is associated with a women's health disorder or condition (e.g., a women's health disorder or condition described herein). In some embodiments, the neuroendocrine disorder or dysfunction is associated with a women's health disorder or condition is polycystic ovary syndrome.

Symptoms of neuroendocrine disorder include, but are not limited to, behavioral, emotional, and sleep-related symptoms, symptoms related to reproductive function, and somatic symptoms; including but not limited to fatigue, poor memory, anxiety, depression, weight gain or loss, emotional lability, lack of concentration, attention difficulties, loss of libido, infertility, amenorrhea, loss of muscle mass, increased belly body fat, low blood pressure, reduced heart rate, hair loss, anemia, constipation, cold intolerance, and dry skin.

### Movement Disorders

Also provided herein are methods, therapeutic agents and compositions for treating a movement disorder. Movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea, tremor), dyskinesia (including tics such as complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis), essential tremor, Stiff-Person syndrome, spasticity, Freidrich's ataxia, Cerebellar ataxia, dystonia, Tourette Syndrome, Fragile X-associated tremor or ataxia syndromes, drug-induced or medication-induced Parkinsonism (such as neuroleptic-induced acute akathisia, acute dystonia, Parkinsonism, or tardive dyskinesia, neuroleptic malignant syndrome, or medication-induced postural tremor), etc.

Tremor is an involuntary, at times rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occurs in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease. Rubral tremor is characterized by coarse slow tremor which can be present at rest, at posture, and with intention. The tremor is associated with conditions that affect the red nucleus in the midbrain, classical unusual strokes.

Ataxia includes cerebellar ataxia (McLeod neuroacanthocytosis syndrome (MLS), levodopa-induced dyskinesia.

### Sleep Disorders

Also provided herein are methods, therapeutic agents and compositions for treating a sleep disorder. The term “sleep disorder” is meant to refer to generally any abnormal sleeping pattern. Some sleep disorders are serious enough to interfere with normal physical, mental, social and emotional functioning. Sleep disorders are broadly classified into insomnia, dyssomnias, parasomnias, circadian rhythm sleep disorders involving the timing of sleep, and other disorders including ones caused by medical or psychological conditions and sleeping sickness. Non-limiting examples of sleep disorders include circadian rhythm abnormality, insomnia, parasomnia, sleep apnea syndrome, narcolepsy and hypersomnia, rapid eye movement behavior disorder, restless legs syndrome, periodic leg movements of sleep, obstructive sleep apnea, central sleep apnea, snoring, nightmares, sleep terrors, sleepwalking, confusional arousals, sleep paralysis, sleep eating disorder, or narcolepsy (See, for example, C G Goetz (editor), Textbook of Clinical Neurology, 3rd Edition, 2007, Chapter 54).

### Seizure

Also provided herein are methods, therapeutic agents and compositions for treating a seizure. A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus. There are six types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

Seizure described herein can include focal seizures with either motor (automatisms, atonic, clonic, epileptic spasms, hyperkinetic, myoclonic, and tonic) or non-motor (autonomic, behavioral arrest, cognition, emotional, and sensory) onset, generalized seizures with either motor (tonic-clonic, clonic, myoclonic, myoclonic-tonic-clonic, myoclonic-atonic, atonic, epileptic spasms) or non-motor (absence) onset, seizures with unknown motor (tonic-clonic, epileptic spasms) or non-motor (behavioral arrest) onset; seizures associated with clinical syndromes, such as Dravet syndrome, Rett syndrome, Lennox Gasteau syndrome, Tuberous sclerosis, Angelmans syndrome, catamenial epilepsy.

### Neuroinflammatory Disorders

Also provided herein are methods, therapeutic agents and compositions for treating a neuroinflammatory disorder. The term “neuroinflammatory disorder” designates a disease having a neuroinflammation component such as, in particular a neurodegenerative, autoimmune, infectious, toxic or traumatic disorder, where inflammatory component could be aetiological or pathology-exacerbating factor. Said neurodegenerative, autoimmune, infectious, toxic or traumatic diseases with inflammatory component include multiple sclerosis (MS), Alzheimer's disease (AD), Parkinson's disease (PD), Amyotrophic lateral sclerosis (ALS), Acute disseminated encephalomyelitis (ADEM) and Neuromyelitis optica (NMO).

### Analgesia/Sedation

Also provided herein are methods, therapeutic agents and compositions for inducing anesthesia, analgesia and sedation. Anesthesia is a pharmacologically induced and reversible state of amnesia, analgesia, loss of responsiveness, loss of skeletal muscle reflexes, decreased stress response, or all of these simultaneously. These effects can be obtained from a single drug which alone provides the correct combination of effects, or occasionally with a combination of drugs (e.g., hypnotics, sedatives, paralytics, analgesics) to achieve very specific combinations of results. Anesthesia allows patients to undergo surgery and other procedures without the distress and pain they would otherwise experience.

Sedation is the reduction of irritability or agitation by administration of a pharmacological agent, generally to facilitate a medical procedure or diagnostic procedure.

Sedation and analgesia include a continuum of states of consciousness ranging from minimal sedation (anxiolysis) to general anesthesia.

Minimal sedation is also known as anxiolysis. Minimal sedation is a drug-induced state during which the patient responds normally to verbal commands. Cognitive function and coordination may be impaired. Ventilatory and cardiovascular functions are typically unaffected.

Moderate sedation/analgesia (conscious sedation) is a drug-induced depression of consciousness during which the patient responds purposefully to verbal command, either alone or accompanied by light tactile stimulation. No interventions are usually necessary to maintain a patient airway. Spontaneous ventilation is typically adequate. Cardiovascular function is usually maintained.

Deep sedation/analgesia is a drug-induced depression of consciousness during which the patient cannot be easily aroused, but responds purposefully (not a reflex withdrawal from a painful stimulus) following repeated or painful stimulation. Independent ventilatory function may be impaired and the patient may require assistance to maintain a patient airway. Spontaneous ventilation may be inadequate. Cardiovascular function is usually maintained.

General anesthesia is a drug-induced loss of consciousness during which the patient is not arousable, even to painful stimuli. The ability to maintain independent ventilatory function is often impaired and assistance is often required to maintain a patient airway. Positive pressure ventilation may be required due to depressed spontaneous ventilation or drug-induced depression of neuromuscular function. Cardiovascular function may be impaired.

Sedation in the intensive care unit (ICU) allows the depression of patients' awareness of the environment and reduction of their response to external stimulation. It can play a role in the care of the critically ill patient, and encompasses a wide spectrum of symptom control that will vary between patients, and among individuals throughout the course of their illnesses. Heavy sedation in critical care has been used to facilitate endotracheal tube tolerance and ventilator synchronization, often with neuromuscular blocking agents.

In some embodiments, sedation (e.g., long-term sedation, continuous sedation) is induced and maintained in the ICU for a prolonged period of time (e.g., 1 day, 2 days, 3 days, 5 days, 1 week, 2 week, 3 weeks, 1 month, 2 months). Long-term sedation agents may have long duration of action. Sedation agents in the ICU may have short elimination half-life.

Procedural sedation and analgesia, also referred to as conscious sedation, is a technique of administering sedatives or dissociative agents with or without analgesics to induce a state that allows a subject to tolerate unpleasant procedures while maintaining cardiorespiratory function.

### Sensory Deficit Disorders

Also provided herein are methods, therapeutic agents and compositions for treating a sensory deficient disorder. Sensory processing disorder is a condition in which the brain has trouble receiving and responding to information that comes in through the senses. Non-limiting examples of sensory deficit disorders include tinnitus, synesthesia, hearing impairment and loss. Pathological brain function in sensory-deficit disorders has been associated with nicotinic cholinergic transmission particularly through oc7 receptors (Freedman R et al., Biol. Psychiatry, 1995, 38, 22-33; Tsuang D W et al., Am. J. Med. Genet., 2001, 105, 662-668; Carson R et al; Neuromolecular, 2008, Med. 10, 377-384; Leonard S et al., Pharmacol. Biochem. Behav., 2001, 70, 561-570; Freedman R et al., Curr. Psychiatry Rep., 2003, 5, 155-161; Cannon T O et al; Curr. Opin. Psychiatry, 2005, 18, 135-140). A defective pre-attention processing of sensory information is understood to be the basis of cognitive fragmentation in schizophrenia and related neuropsychiatric disorders (Leiser S C et al., Pharmacol. Ther., 2009, 122, 302-31 1). Genetic linkage studies have traced sharing of the oc7 gene locus for several affective, attention, anxiety and psychotic disorders (Leonard S et al., Pharmacol. Biochem. Behav., 2001, 70, 561-570; Suemaru K et al., Nippon Yakurigaku Zasshi, 2002, 1 19, 295-300).

### Neuroprotection, Glaucoma, Metabolic Disorders

Also provided herein are methods, therapeutic agents and compositions for treating a neuroprotection disorder, a metabolic disorder, such as glaucoma. The term “glaucoma” refers to an ocular disease in which the optic nerve is damaged in a characteristic pattern. This can permanently damage vision in the affected eye and lead to blindness if left untreated. It is normally associated with increased fluid pressure in the eye (aqueous humor). The term ocular hypertension is used for patients with consistently raised intraocular pressure (TOP) without any associated optic nerve damage. Conversely, the term normal tension or low tension glaucoma is used for those with optic nerve damage and associated visual field loss but normal or low TOP. The nerve damage involves loss of retinal ganglion cells in a characteristic pattern. There are many different subtypes of glaucoma, but they can all be considered to be a type of optic neuropathy. Raised intraocular pressure (e.g., above 21 mmHg or 2.8 kPa) is the most important and only modifiable risk factor for glaucoma. However, some may have high eye pressure for years and never develop damage, while others can develop nerve damage at a relatively low pressure. Untreated glaucoma can lead to permanent damage of the optic nerve and resultant visual field loss, which over time can progress to blindness.

### Female Sexual Dysfunction

Also provided herein are methods, therapeutic agents and compositions for treating a female sexual disorder. “Female sexual dysfunctions” or “female sexual disorders” are defined in The Diagnostic and Statistical Manual of Mental Disorders (“DSM”), and include the following three categories: (1) Genitopelvic pain/penetration disorder; (2) Sexual interest/arousal disorder; and (3) Female orgasmic disorder. All of these afflictions are common and known to significantly reduce the quality of life for millions of women and their sexual partners. In the most current version of DSM, (DSM-V), older terminologies, both Hypoactive Sexual Desire Disorder (HSDD) and Female Arousal Disorder were merged into a single category of female sexual disorder now called Sexual Interest/Arousal Disorder (SIAD). Similarly, the formerly separate dyspareunia and vaginismus disorders are now collectively called Genitopelvic Pain/Penetration Disorder (GPPD). The terminology Female Orgasmic Disorder (FOD) remains unchanged.

The DSM-V defines genitopelvic pain/penetration disorder, (GPPD) as difficulty in vaginal penetration, marked vulvovaginal or pelvic pain during penetration, or attempt at penetration (dyspareunia), fear or anxiety about pain in anticipation of, during, or after penetration, and tightening or tensing of pelvic floor muscles during attempted penetration (vaginismus). For many women, however, pain may occur outside the context of penetration or sexual intercourse. For example, pain or discomfort may occur during any manipulation of their external genitalia (a condition known as vulvodynia). Such pain or discomfort may be due to Vulvovaginal atrophy (VVA), including thinning of the vulvovaginal epithelium and a lack of lubrication and/or may be characterized as vulvodynia.

This condition may manifest itself as primary vestibulodynia (i.e. pain with first attempted tampon and/or intercourse) or secondary vestibulodynia (i.e. development of vulvar pain following previously painless tampon use and/or intercourse).

Another contributing cause of genital pain/penetration disorder (GPPD) is known as vestibulodynia, vulvodynia and/or vestibulitis. This affliction may be seen in up to 15% of all premenopausal women sometime in their lifetime. It is also a very common affliction of postmenopausal women unrelated to VVA.

Female vestibulodynia may be generally described as a disorder of unknown etiology where there is localized provoked vulvar pain upon penetration of the vagina. There is also tenderness to touch around the vaginal opening (vestibule) during normal self or partner's manual sexual contact or during a health professional's physical examination. The entire area around the vaginal introitus (vulvodynia) can be affected but the experienced discomfort or pain is most commonly pronounced in the localized area of the posterior forchette (vestibulodynia). The affected tissue in the vestibule has increased nerve endings and signs of inflammation and is typically painful. It occurs in women of all ages.

The two other disorders of female sexual dysfunction are: sexual interest/arousal disorder (SIAD) and female orgasmic disorder (FOD). Sexual Interest/Arousal Disorder (“SIAD”) as specified in the DSM refers to “the persistent or recurrent inability to attain or to maintain sufficient sexual excitement, which causes personal distress.” In addition to absent or decreased sexual interest, including erotic thoughts or fantasies, there are four criteria that are taken into account to determine whether a woman suffers from SIAD. A woman has SIAD if she experiences personal distress caused by a decrease or lack of at least three of the following four criteria: 1) initiation of sexual activity or responsiveness to a partner's attempts to initiate it, 2) excitement and pleasure, 3) response to sexual cues, and 4) sensations during sexual activity, whether genital or non-genital. Again, three of the foregoing criteria are required for diagnosis. It may be expressed generally as lack of subjective excitement or lack of genital (lubrication/swelling) or other somatic responses.

Female orgasmic disorder, (FOD) as defined in the DSM, is the absence (anorgasmia), infrequency or delay of orgasm, and/or reduced intensity of said orgasm. Such orgasmic dysfunction may also occur when a woman has difficulty reaching orgasm, even when sexually aroused with sufficient sexual stimulation. Many women have difficulty reaching orgasm with a partner, or during masturbation, even after ample sexual stimulation. Female Orgasmic Disorder (FOD) affects approximately one in three women.

It can be difficult to determine the particular underlying cause of Female Orgasmic Disorder (FOD). Women may have difficulty reaching orgasm due to one or more physical, emotional, and/or psychological factors. Contributing factors include: older age, medical conditions, such as diabetes, a history of gynecological surgeries, such as a hysterectomy, the use of certain medications, particularly selective serotonin reuptake inhibitors (SSRIs), mental health conditions, such as depression or anxiety, stress, societal negative stereotypes of women's sexuality, lack of adequate or effective sexual stimulation, etc. Sometimes, a combination of these factors can make achieving an orgasm difficult or not possible.

The inability to orgasm can lead to distress, which may make it even more difficult to achieve orgasm in the future. The main symptom of orgasmic disorder is the inability to achieve sexual climax. Women with female orgasmic disorder (FOD) may have difficulty achieving orgasm during either sexual intercourse or during masturbation. For many women, having unsatisfying orgasms, less intense orgasms, or taking longer than desirable to reach climax are common symptoms of FOD that lead to emotional distress.

According to certain embodiments, transdermal compositions described herein are for the treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, and cancer related or other end-of-life psychological distress.

Further indications are cognitive disorders. The term “cognitive disorder” shall refer to anxiety disorders, delirium, dementia, amnestic disorders, dissociative disorders, eating disorders, mood disorders, schizophrenia, psychotic disorders, sexual and gender identity disorders, sleep disorders, somatoform disorders, acute stress disorder, obsessive-compulsive disorder, panic disorder, posttraumatic stress disorder, specific phobia, social phobia, substance withdrawal delirium, Alzheimer's disease, Creutzfeldt-Jakob disease, head trauma, Huntington's disease, HIV disease, Parkinson's disease, Pick's disease, learning disorders, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, pica, rumination disorder, tic disorders, chronic motor or vocal tic disorder, Tourette's disorder, elimination disorders, encopresis, enuresis, selective mutism, separation anxiety disorder, dissociative amnesia, depersonalization disorder, dissociative fugue, dissociative identity disorder, anorexia nervosa, bulimia nervosa, bipolar disorders, schizophreniform disorder, schizoaffective disorder, delusional disorder, psychotic disorder, shared psychotic disorder, delusions, hallucinations, substance-induced psychotic disorder, orgasmic disorders, sexual pain disorders, dyspareunia, vaginismus, sexual dysfunction, paraphilias, dyssomnias, breathing-related sleep disorder, circadian rhythm sleep disorder, hypersomnia, insomnia, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, alcohol related disorders, amphetamine related disorders, caffeine related disorders, cannabis related disorders, cocaine related disorders, hallucinogen related disorders, inhalant related disorders, nicotine related disorders, opioid related disorders, phencyclidine-related disorder, abuse, persisting amnestic disorder, intoxication, withdrawal.

The term “bipolar and clinical disorders” shall refer to adjustment disorders, anxiety disorders, delirium, dementia, amnestic and other cognitive disorders, disorders usually first diagnosed in infancy (e.g.), childhood, or adolescence, dissociative disorders (e.g. dissociative amnesia, depersonalization disorder, dissociative fugue and dissociative identity disorder), eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder (e.g. acute stress disorder, posttraumatic stress disorder), panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the disclosure), that are serviceable as therapies for treating, preventing or improving conditions, states and disease as provided in the disclosure. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the disclosure provides for blister packs, clamshells or trays comprising a composition (e.g., a (the multi-ingredient combination of drugs of the disclosure) combination of active ingredients) of the disclosure. Blister packs, clamshells or trays can be designed to be non-reclosable, so consumers can tell if a package has already opened. They are used to package for sale goods where product tampering is a consideration, such as the agents of the disclosure. In one aspect, a blister pack of the disclosure comprises a molded PVC base, with raised areas (the “blisters”) to contain the tablets, pills, etc. comprising the combinations of the disclosure, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the disclosure are contained in-between a card and clear PVC. The PVC can be transparent so the item (patch, pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (patch, pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed patches, pills, tablets, geltabs, etc., the card has a perforated window for access. In one aspect, more secure blister packs, e.g., for items such as patches, pills, tablets, geltabs, etc. of the disclosure are used, and they can comprise of two vacuum-formed PVC sheets meshed together at the edges, with the informative card inside.

In one aspect, blister packaging comprises at least two components (e.g., is a multi-ingredient combination of drugs of the disclosure): a thermoformed “blister” which houses the product (e.g., a combination of the disclosure), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the disclosure can comprise the packaging of the therapeutic drug combinations of the disclosure, alone or in combination, as “blister packages” or as a plurality of packets, including as lidded blister packages, lidded blister or blister card or packets, or a shrink wrap.

In one aspect, any of the disclosure products of manufacture, including kits or blister packs, include memory aids to help remind patients when and how to take the agents of the disclosure.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the disclosure. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

This Example describes the preparation of a patch or semisolid formulation, which must give a blood level (±20%) bioequivalent to oral psilocybin. Initially, a transdermal formulation will be prepared containing a dose of psilocybin based on the in-vitro permeability flux profile obtained from Franz-diffusion cells, the dose will be adjusted to obtain desired blood level (±20%) bioequivalent to oral 10 mg/day psilocybin. Different approaches will be implemented (e.g. change in drug loading dose, combination of solvents/enhancers etc.) to prepare a transdermal formulation which can deliver target therapeutic blood level up to 15 or 30 days.

### Example 2

Below is a description of the experimental procedure, utilized for development and optimization of transdermal matrix patch or transdermal semisolid formulation containing psilocybin lone or psilocin alone, or a combination of psilocybin and psilocin. Exemplary formulations are set forth in Table 1:

TABLE 1

| | PSI 1 | PSI 2 | PSI 3 | PSI 4 |
|---|---|---|---|---|
| Excipients | (% w/w) | (% w/w) | (% w/w) | (% w/w) |
| Psilocybin/psilocin | 0.1-20% | 0.1-20% | 0.1-20% | 0.1-20% |
| Enhancers | | 0.1-20% | | 0.1-20% |
| Solvents | | | 0.1-20% | 0.1-20% |
| Adhesive/Polymers | 80-99.9% | 50-99.8% | 50-99.8% | 30-99.7% |

The transdermal formulation and/or topical formulation of the disclosure may comprise solvents known to those skilled in the art either alone or in combinations thereof without any limitation to following like alcohol Ci-Cao such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butylene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, sesame oil water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane), acids such as but not limited to acetic acid, lactic acid, levulinic acid, bases and others, pentane, dimethylformamide, butane, lipids. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 95% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

The transdermal formulation and/or topical formulation of the disclosure may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives, Cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 9′71p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.,), acrylic pressure sensitive adhesives such as but not limited to (duro-tak 87-2156, duro-tak 387-2287, duro-tak 87-9301, duro-tak 387-2051 etc.), polyisobutylene such as but not limited to (polyisobutylene low molecular weight, polyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 85%, and about 90% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymer and/or pressure sensitive adhesive polymers will represent approximately 1 wt % to 75 wt %, preferably 2 wt % to 30 wt %, more preferably 5 wt. % to 20 wt. % of the formulation, and more preferably in the range of 0.1% 80% w/w or w/v.

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation, and more preferably in the range of 0.01%-95% w/w or w/v.

All of the components from Table 1, with the exception of the Psilocybin, were mixed together with stirring for 18 hours. Next, the Psilocybin was added into the excipient mixture to prepare the final transdermal formulations.

### Example 3

The following steps are provided using composition PSI 1 as an example for preparing a transdermal patch. The above ingredients are blended by stirring until uniform blend is achieved then, using a commercial benchtop spreader, the matrix is evenly spread onto an 8×14 inch sheet of release liner (such as 3M 9744) to achieve a desired coat weight of laminate.

The sheet is then placed in an oven to dry out the volatile component of the adhesive solvent. A backing membrane (such as 3M 9730 NR film) with low permeability to oxygen to inhibit photo and oxidative degradation is then carefully applied by hand to avoid formation of bubbles and voids. A circular die (1.5 inches diameter) is used to cut patches (7 sqcm) for subsequent studies. After drying, the drug adhesive matrix has a surface density of 5-30 mg/sqcm, containing psilocybin in 0.1-20% w/w.

### Example 4

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells comprising a cylindrical donor compart and a separate water jacketed cylindrical receptor compartment with the volume of 13 mL. The cadaver skin was clamped between the two compartments with the dermis side facing toward the receptor compartment. The donor compartment was filled with the transdermal Psilocybin/Psilocin formulations prepared as described above. The receptor compartment was filled with receptor medium, held at constant temperature, and constantly stirred to collect the Psilocybin as it diffuses through the skin and into receptor compartment. It is important to confirm that the receptor fluid is always in contact with the skin. The receptor compartment was emptied as desired for assay of Psilocybin and replaced with fresh receptor solution. In order to maintain the sink condition in the receptor compartment, it is important to keep the Psilocybin concentration in the receptor compartment less than 10% of its solubility.

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-glycerides, polyglyceryl-3-dioleate, diethylene glycol monoethyl ether, propylene glycol monolaurate type I, polyglyceryl-3-dioleate, caprylocaproyl polyoxyl-8 glycerides etc, cyclodextrins and others. More preferably in the range of 0.01% 95% w/w or w/v. In exemplary embodiments, formulations of the disclosure may comprise solubilizers, surfactants, emulsifying agents, dispersing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, and more preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, etc.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation, and more preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, and more preferably in the range of 0.01%-50% w/w or w/v.

### Example 10

The transdermal formulation and/or topical formulation of the disclosure may be formulated in ointment and/or cream base and/or gel and/or film forming formulation and/or transdermal matrix formulation and/or drug-in-adhesive matrix patch and/or matrix patch known to those skilled in the art.

### Example 11

Materials to make the transdermal delivery system of the disclosure in patch form known to those skilled in the art, for example, such as but not limited to a multilayer transdermal matrix system. reservoir patch, matrix patch, drug in adhesives, film forming formulation, micro-dosing transdermal patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of active agent, such as for example, CBD, THC, psilocybin, psilocin, lysergic acid diethylamine (LSD), and/or ibogaine, and derivatives of these compounds, alone or in combinations thereof in human plasma required for treating and/or preventing pain and/or inflammation. Therapeutically effective active agent such as CBD, THC, psilocybin, psilocin, lysergic acid diethylamine (LSD), and/or ibogaine, and derivatives of these compounds dosages refers to the therapeutic concentration of in human plasma required for treating and/or preventing pain and/or inflammation. Furthermore, the precise therapeutic effective dose of such as psilocybin, psilocin, lysergic acid diethylamine (LSD), and/or ibogaine, and derivatives of these compounds in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In yet another embodiment, the transdermal formulation and/or topical formulation and/or transdermal delivery system of the disclosure may deliver at least therapeutic effective dose of such as CBD, THC, psilocybin, psilocin, lysergic acid diethylamine (LSD), and/or ibogaine, and derivatives of these compounds. Therapeutically effective doses of active agents such as CBD, THC, psilocybin, psilocin, lysergic acid diethylamine (LSD), and/or ibogaine, and derivatives of these compounds refers to the therapeutic concentration of active agent in human plasma required for the treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, and cancer related or other end-of-life psychological distress in a patient.

The transdermal formulation or transdermal patch of active agents such as CBD, THC, psilocybin, psilocin, lysergic acid diethylamine (LSD), and/or ibogaine, and derivatives of these compounds can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

Pressure Sensitive Adhesive Formulation:

| Ingredients | % W/W |
|---|---|
| Active component | 0.1%-30% |
| Solvent | 1%-40% |
| Permeation Enhancers | 1%-40% |
| Pressure sensitive adhesive | 20%-90% |
| Polymers | 2%-50% |

The present formulation is not deemed to be limited thereto.

### Example 13

Formulations of drug in adhesive matrix patch

| Component | % W/W |
|---|---|
| Active component (THC and/or CBD) | 0.5%-30% |
| Solvent | 2%-30% |
| Permeation enhancer | 2%-30% |
| Pressure sensitive adhesive polymer | 20%-80% |
| Polymer | 1%-10% |

### Example 14

Formulations of drug in adhesive matrix patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

### Example 15

Synthetic delta-9-thc (THC) and cannabidiol (CBD) formulations for transdermal delivery ((Formulation Nos. 001, 002, 003, 004, and 005) were prepared by mixing ingredients as shown in Table 2:

TABLE 2
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 8.0 | 8.0 | 7.7 | 6.9 | 6.4 |
| THC | 8.0 | 8.0 | 7.7 | 6.9 | 6.4 |
| Ethanol | 42.7 | 37.3 | 37.0 | 33.0 | 25.4 |
| Propylene | 40.0 | 40.0 | 37.0 | 33.0 | 30.5 |
| Glycol | | | | | |
| Isopropyl | | | 4.7 | — | — |
| Palmitate | | | | | |
| DMSO | | | | 13.8 | 12.7 |
| Oleic Acid | | 5.3 | 4.7 | 5.5 | 5.1 |
| NMP | | | | — | 12.7 |

Abbreviations: THCH = delta-9-THC;
CBD = Cannabidiol;
NMP: N-methyl Pyrrolidone.

All of the components from Table 2, with the exception of the CBD and THC, were mixed together with stirring for 18 hours. Next, the CBD and THC were added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD and THC formulations prepared as described above. The receptor compartment was filled with receptor medium, held at constant temperature, and constantly stirred to collect the CBD and THC as it diffuses through the skin and into receptor compartment. It is important to confirm that the receptor fluid is always in contact with the skin. The receptor compartment was emptied at 24 hr intervals for assay of CBD and THC and replaced with fresh receptor solution. In order to maintain the sink condition in receptor compartment, it is important to keep the CBD and THC concentration in receptor compartment less than 10% of its solubility. The experimental conditions are provided in Table 3:

TABLE 3
Experimental Condition for In-vitro Permeability testing

| | |
|---|---|
| Receiving Media | De-ionized water + 0.5% Brij-O(20) + |
| | 0.01% Sodium Azide |
| Receiving Media Volume (mL) | 13 |
| Sample Volume (mL) | 13 |
| Sampling Interval (hr) | 24, 48, 72 |
| Franz-cell diffusion area (sqcm) | 1.76 |
| Membrane Type | Human Cadaver Skin |

Flux of CBD and THC through the human cadaver skin was measured for a minimum period of 72 Hrs (3 days) and results of the flux measurement are provided in Table 4 and 5.

TABLE 4
CBD Flux Results

| | 009 | 010 | 011 | 012 | 013 |
|---|---|---|---|---|---|
| Average Flux (0-24 hr) | 0.53 | 0.86 | 1.10 | 1.39 | 1.01 |
| (ug/sqcm/hr) | (23.6%) | (27.0%) | (20%) | (19.9%) | (5.1%) |
| Average Flux (24-48 hr) | 0.92 | 1.16 | 1.21 | 1.44 | 1.21 |
| (ug/sqcm/hr) | (9.6%) | (14.4%) | (18.3%) | (1.65%) | (27.3%) |
| Average Flux (48-72 hr) | 0.52 | 0.86 | 0.71 | 1.27 | 1.01 |
| (ug/sqcm/hr) | (2.44%) | (12.9%) | (13%) | (16.3%) | (27.5%) |
| Average Flux (0-72 hr) | 0.66 | 0.96 | 1.01 | 1.37 | 1.08 |
| (ug/sqcm/hr) | | | | | |

TABLE 5
THC Flux Results

| | 009 | 010 | 011 | 012 | 013 |
|---|---|---|---|---|---|
| Average Flux (0-24 hr) | 0.0 | 0.28 | 0.62 | 0.73 | 0.60 |
| (ug/sqcm/hr) | | (89%) | (20.4%) | (24.5%) | (5.1%) |
| Average Flux (24-48 hr) | 0.39 | 0.65 | 0.78 | 0.85 | 0.80 |
| (ug/sqcm/hr) | (87%) | (20.8%) | (19.4%) | (8.7%) | (32.3%) |
| Average Flux (48-72 hr) | 0.0 | 0.47 | 0.43 | 0.73 | 0.58 |
| (ug/sqcm/hr) | | (20.3%) | (32%) | (20.2%) | (40%) |
| Average Flux (0-72 hr) | 0.13 | 0.47 | 0.61 | 0.77 | 0.66 |
| (ug/sqcm/hr) | | | | | |

While the invention has been described in detail and with reference to specific examples thereof, it will be apparent to one skilled in the art that various changes and modifications can be made therein without departing from the spirit and scope thereof.

1. A transdermal and/or topical pharmaceutical composition comprising:
at least one active agent selected from the group consisting of:
about 0.1% to about 50% of an active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, naturally derived forms thereof, active metabolites thereof, polymorph thereof, solid solution thereof, coated form thereof, and combinations thereof,
further wherein the pharmaceutical composition comprises:
about 10% to about 99.9% of an adhesive and/or polymer;
optionally, about 0.1% to about 99% of a permeation enhancer;
optionally, about 0.1% to about 99% of a solvent,
wherein said pharmaceutical composition will have no or minimal hallucinogenic or psychoactive effect in a patient to whom the pharmaceutical composition is applied.

2. The pharmaceutical composition of claim 1, wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL.

3. The pharmaceutical composition of claim 1, wherein the pharmaceutical formulation provides a dose of active agent to a patient equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, 500 microgram/day, about 0.001 mg/day, 0.01 mg/day, 0.025 mg/day. 0.05 mg/day, 0.1 mg/day, 0.25 mg/day, 0.5 mg/day, 1 mg/day, 10 mg/day, or 25 mg/day.

4. The pharmaceutical composition of claim 1, wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, naturally derived forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

5. The pharmaceutical composition of claim 1, wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

6. A pharmaceutical composition of claim 1 comprising one or more active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for transdermal delivery.

7. A pharmaceutical composition of claim 1 comprising one or more active agent selected from the group consisting of tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorph thereof, solid solution thereof, coated form thereof, and combinations thereof, in a dosage form for topical delivery.

8. The pharmaceutical composition of claim 1 wherein said CBD, THC, psilocybin, psilocin, lysergic acid diethylamine (LSD), and/or ibogaine, the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof, is produced by a natural route.

9. The pharmaceutical composition of claim 1 wherein said tetrahydrocannabinol (THC), cannabidiol (CBD), psilocybin, psilocin, lysergic acid diethylamine (LSD), and/or ibogaine, the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof, is produced by a synthetic route.

10. The pharmaceutical composition of claim 1 formulated as transdermal liquid formulation, transdermal semisolid formulation, transdermal gel formulation, or transdermal polymer matrix formulation, transdermal adhesive matrix formulation, transdermal film forming gel, transdermal film forming formulation, a multilayer transdermal matrix system, or transdermal drug-in-adhesive matrix formulation.

11. The pharmaceutical composition of claim 1 formulated as a topical liquid formulation, topical semi solid formulation, topical gel formulation, topical polymer matrix formulation, topical adhesive matrix formulation, topical film forming gel formulation, or topical film forming spray formulation.

12. The pharmaceutical composition of claim 1 which is formulated as a patch.

13. The pharmaceutical composition of claim 1 which is formulated as two or more patches.

14. The pharmaceutical composition of claim 1 wherein the two or more patches each compromise the same active agent.

15. The pharmaceutical composition of claim 1 wherein the two or more patches each comprise different active agents.

16. The pharmaceutical composition of claim 1 wherein the two or more patches each comprise the same or different active agents.

17. The pharmaceutical composition of claim 1 which is formulated as a transdermal patch.

18. The pharmaceutical composition of claim 1 formulated as a transdermal patch, wherein the transdermal patch is selected from the group consisting of a reservoir patch, a microreservoir patch, a micro-dosing patch, a matrix patch, a drug in adhesive patch, a pressure sensitive adhesive patch, extended-release transdermal film a liquid reservoir system, a microreservoir patch, a mucoadhesive patch, multilayer transdermal matrix system, and combinations thereof.

19. The pharmaceutical composition of claim 1, which is formulated as a topical patch.

20. The pharmaceutical composition of claim 1 formulated as a topical patch, wherein the topical patch is selected from the group consisting of a reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, multilayer transdermal matrix system, and combinations thereof.

21. The pharmaceutical composition of claim 1 which is formulated as metered dose transdermal gel, metered dose transdermal spray, a film forming gel, a film forming spray, or a meter-dose aerosol.

22. The pharmaceutical composition of claim 1 formulated as microneedles.

23. The pharmaceutical composition of claim 1 formulated as a liquid formulation, transdermal semisolid formulation, or transdermal polymer matrix formulation, transdermal adhesive matrix formulation, film forming gel formulation, film forming spray formulation.

24. The pharmaceutical composition of claim 1 further comprising at least one additional active agent selected from the group consisting of THC, CBD, psilocybin, psilocin, lysergic acid diethylamine (LSD), and/or ibogaine, antidepressant drug, NSAIDS, anticonvulsants drug, corticosteroid drug, pain relievers, lidocaine, menthol, capsaicin, methyl salicylate, lidocaine, capsaicin, Tricyclic Antidepressants, amitriptyline, imipramine, nortriptyline, desipramine, doxepin, SNRIs and SSRIs, duloxetine, venlafaxine, fluoxetine, milnacipran, diclofenac, aspirin, naproxen, ibuprofen, ketoprofen, celecoxib, meloxicam, acetaminophen, cox-2 inhibitors, celecoxib, anticonvulsants, carbamazepine, gabapentin, lamotrigine, pregabalin, oxcarbazepine, lamotrigine, valproic acid, menthol, camphor, methyl salicylate, salicylates, corticosteroid drugs, triamcinolone, methylprednisolone, cortisone, prednisone, dexamethasone, opioids, and combinations thereof.

25. The pharmaceutical composition of claim 1 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

26. The pharmaceutical composition of claim 1 wherein the adhesive is selected from the group consisting of pressure sensitive adhesives, silicone polymers, bio psa 4302, bio-psa 4202, acrylic pressure sensitive adhesives, duro-tak 87-2156, duro-tak 387-2287, duro-tak 87-9301, duro-tak 387-2051, polyisobutylene, polyisobutylene low molecular weight, polyisobutylene medium molecular weight, polyisobutylene 35000 mw, acrylic copolymers, rubber based adhesives, hot melt adhesives, styrene-butadiene copolymers, bentonite, all water and/or organic solvent swellable polymers and combinations thereof.

27. The pharmaceutical composition of claim 1 wherein said polymer is present and is selected from the group consisting of natural polymers, polysaccharides. agar, alginic acid and derivatives, Cassia tora, collagen, gelatin, gellum gum, guar gum, pectin, potassium cargeenan, sodium carageenan, tragacanth, xantham, gum copal, chitosan, resin, semisynthetic polymers, cellulose, methylcellulose, ethyl cellulose, carboxymethyl cellulose, hydroxylpropyl cellulose, hydroxylpropylmethyl cellulose, synthetic polymers, carboxyvinyl polymers, carbomers, carbopol 940, carbopol 934, carbopol 9′71p NF, polyethylene, clays, silicates, bentonite, silicon dioxide, polyvinyl alcohol, acrylic polymers (eudragit), acrylic acid esters, polyacrylate copolymers, polyacrylamide, polyvinyl pyrrolidone homopolymer, polyvinyl pyrrolidone copolymers, PVP, Kollidon 30, poloxamer, isobutylene, ethyl vinyl acetate copolymers, natural rubber, synthetic rubber, and combinations thereof.

28. The pharmaceutical composition of claim 1 wherein said permeation enhancer is present, and is selected from the group consisting of dimethylsulfoxide, dimethyl acetamide, dimethylformamide, decymethylsulfoxide, dimethylisosorbide, azone, pyrrolidones, N-methyl-2-pyrrolidone, 2-pyrrolidon, esters, fatty acid esters, propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate, fatty acids, capric acid, caprylic acid, lauric acid, oleic acid, myristic acid, linoleic acid, stearic acid, palmitic acid, alcohols, fatty alcohols, glycols, oleyl alcohol, nathanol, dodecanol, propylene glycol, glycerol, ethers, alcohol, diethylene glycol monoethyl ether, urea, triglycerides, triacetin, polyoxyethylene fatty alcohol ethers, polyoxyethylene fatty acid esters, esters of fatty alcohols, essential oils, surfactant type enhancers, brij, sodium lauryl sulfate, tween, polysorbate, terpene, terpenoids, and combinations thereof.

29. The pharmaceutical composition of claim 1 wherein said solvent is present, and is selected from the group consisting of methanol, ethanol, isopropyl alcohol, butanol, propanol, polyhydric alcohols, glycols, propylene glycol, polyethylene glycol, dipropylene glycol, hexylene glycol, butylene glycol, glycerine, derivative of glycols, pyrrolidone, N methyl 2-pyrrolidone, 2 pyrrolidone, sulfoxides, dimethyl sulfoxide, decymethylsulfoxide, dimethylisosorbide, mineral oils, vegetable oils, sesame oil water, polar solvents, semi polar solvents, non polar solvents, volatile chemicals, ethanol, propanol, ethyl acetate, acetone, methanol, dichloromethane, chloroform, toluene, IPA, hexane, acids, acetic acid, lactic acid, levulinic acid, bases, pentane, dimethylformamide, butane, lipids, and combinations thereof.

30. The pharmaceutical composition of claim 1 which is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

31. The pharmaceutical composition of claim 1 which is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

32. The pharmaceutical composition of claim 1 co-administered with at least one additional an active agent selected from the group consisting of: medications administered for treatment and/or management and/or prevention and/or control of symptoms associated with neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, adjustment disorder, prolonged grief disorder (PGD), and any combination thereof.

33. The pharmaceutical composition of claim 1 indicated for the treatment and/or prevention and/or control of chronic pain, multiple sclerosis, severe depression (treatment resistant), major depressive disorder, obsessive-compulsive disorder, post-traumatic stress disorder, quitting smoking, alcohol addiction, cocaine addiction, opioid addiction, anxiety (stress), adjustment disorder, prolonged grief disorder (PGD), adult ADHD, cluster headaches, and cancer related or other end-of-life psychological distress in a patient.

34. The pharmaceutical composition of claim 1 wherein the pharmaceutical composition provides a continuous, sustained delivery of the pharmaceutical composition to mitigate peak and valley pharmacokinetic behavior of the active agent.

35. The pharmaceutical composition of claim 1 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

36. The pharmaceutical composition of claim 1 wherein the pharmaceutical composition provides a continuous, sustained delivery of the pharmaceutical composition via intravenous or subcutaneous infusion.

37. A method for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient comprising:
selecting a patient in need of treatment and/or prevention and/or control of severe depression (treatment resistant), major depressive disorder, obsessive-compulsive disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress;
topically applying the pharmaceutical composition of claim 1,
thereby treating and/or preventing and/or controlling severe depression (treatment resistant), major depressive disorder, obsessive-compulsive disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in said patient, wherein said patient experiences no or minimal psychoactive or hallucinogenic effects from said transdermal pharmaceutical composition.

38. The method of claim 37 wherein the topical application of a pharmaceutical composition for the treatment and/or prevention and/or control of severe depression (treatment resistant), major depressive disorder, obsessive-compulsive disorder, post-traumatic stress disorder, quitting smoking, alcohol addiction, cocaine addiction, opioid addiction, anxiety (stress), adult ADHD, cluster headaches, adjustment disorder, prolonged grief disorder (PGD), and cancer related or other end-of-life psychological distress in a patient, wherein the transdermal patch is applied at a time period selected from the group consisting of once in a day, once in two days, once in three days, once in four days, once in five days, once in six days, once in a week, once in ten days, and once in fifteen days.

39. The method of claim 37 wherein the pharmaceutical composition is applied to the patient separately, sequentially, or simultaneously.

40. The method of claim 37 further providing a constant rate of delivery of the active components of the transdermal patch over a time period selected from the group consisting of once in a day, once in two days, once in three days, once in four days, once in five days, once in six days, once in a week, once in ten days, and once in fifteen days.

41. The method of claim 37 further providing a steady absorption rates of the active components of the transdermal patch over a time period selected from the group consisting of once in a day, once in two days, once in three days, once in four days, once in five days, once in six days, once in a week, once in ten days, and once in fifteen days.

42. The method of claim 37 further achieving a constant blood serum levels of the active components of the transdermal patch over a time period selected from the group consisting of once in a day, once in two days, once in three days, once in four days, once in five days, once in six days, once in a week, once in ten days, and once in fifteen days.

43. The method of claim 37 further achieving a reduced variability in dosage of the active components of the transdermal patches over a time period selected from the group consisting of once in a day, once in two days, once in three days, once in four days, once in five days, once in six days, once in a week, once in ten days, and once in fifteen days.

44. The method of claim 37 further providing a plasma concentration of the active components of the transdermal patch in a therapeutic range over a time period selected from the group consisting of once in a day, once in two days, once in three days, once in four days, once in five days, once in six days, once in a week, once in ten days, and once in fifteen days.

45. The method of claim 37 further providing a plasma concentration of the active components of the transdermal patch in a therapeutic range of about 0.01 ng/mL to about 500 ng/mL.

46. The method of claim 37 wherein the pharmaceutical composition provides a continuous, sustained delivery of the pharmaceutical composition to mitigate peak and valley pharmacokinetic behavior of the active agent.

47. The method of claim 37 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

48. The method of claim 37 wherein the pharmaceutical composition provides a continuous, sustained delivery of the pharmaceutical composition via intravenous or subcutaneous infusion.

49. A method for the treatment and/or prevention and/or control of chronic pain in a patient comprising:
selecting a patient in need of treatment and/or prevention and/or control of chronic pain;
topically applying the pharmaceutical composition of claim 1, thereby treating, preventing and/or controlling chronic pain in the patient, wherein said patent experiences no or minimal psychoactive or hallucinogenic effects from said transdermal pharmaceutical composition.

50. The method of claim 49, wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

51. The method of claim 49 wherein the topical application of a pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days.

52. The method of claim 49 wherein the pharmaceutical compositions are applied to the patient separately, sequentially or simultaneously.

53. The method of claim 49 wherein the pharmaceutical composition provides a continuous, sustained delivery of the pharmaceutical composition to mitigate peak and valley pharmacokinetic behavior of the active agent.

54. The method of claim 49 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

55. The method of claim 49 wherein the pharmaceutical composition provides a continuous, sustained delivery of the pharmaceutical composition via intravenous or subcutaneous infusion.

56. A method for the treatment and/or prevention and/or control of adjustment disorder in a patient comprising:
selecting a patient in need of treatment and/or prevention and/or control of adjustment disorder;
topically applying the pharmaceutical composition of claim 1,
thereby treating and/or preventing and/or controlling adjustment disorder in the patient, wherein said patent experiences no or minimal psychoactive or hallucinogenic effects from said transdermal pharmaceutical composition.

57. The method of claim 56 wherein the topical application of a pharmaceutical composition for the treatment and/or prevention and/or control of adjustment disorder, wherein the transdermal patch is applied at a time period selected from the group consisting of once in a day, once in two days, once in three days, once in four days, once in five days, once in six days, once in a week, once in ten days, and once in fifteen days.

58. The method of claim 56 further providing a constant rate of delivery of the active components of the transdermal patch over a time period selected from the group consisting of once in a day, once in two days, once in three days, once in four days, once in five days, once in six days, once in a week, once in ten days, and once in fifteen days.

59. The method of claim 56 further providing a steady absorption rates of the active components of the transdermal patch over a time period selected from the group consisting of once in a day, once in two days, once in three days, once in four days, once in five days, once in six days, once in a week, once in ten days, and once in fifteen days.

60. The method of claim 56 further achieving a constant blood serum levels of the active components of the transdermal patch over a time period selected from the group consisting of once in a day, once in two days, once in three days, once in four days, once in five days, once in six days, once in a week, once in ten days, and once in fifteen days.

61. The method of claim 56 further achieving a reduced variability in dosage of the active components of the transdermal patches over a time period selected from the group consisting of once in a day, once in two days, once in three days, once in four days, once in five days, once in six days, once in a week, once in ten days, and once in fifteen days.

62. The method of claim 56 further providing a plasma concentration of the active components of the transdermal patch in a therapeutic range over a time period selected from the group consisting of once in a day, once in two days, once in three days, once in four days, once in five days, once in six days, once in a week, once in ten days, and once in fifteen days.

63. The method of claim 56 wherein the pharmaceutical compositions are applied to the patient separately, sequentially, or simultaneously.

64. The method of claim 56 further providing a plasma concentration of the active components of the transdermal patch in a therapeutic range of about 0.01 ng/mL to about 500 ng/mL.

65. The method of claim 56 wherein the pharmaceutical composition provides a continuous, sustained delivery of the pharmaceutical composition to mitigate peak and valley pharmacokinetic behavior of the active agent.

66. The method of claim 56 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

67. The method of claim 56 wherein the pharmaceutical composition provides a continuous, sustained delivery of the pharmaceutical composition via intravenous or subcutaneous infusion.
